SEQUENCE LISTING (18 sequences)

SEQ ID NO: 1 - protein, 1145 aa, Artificial sequence
  Synthetic Her2 v1 amino acid sequence
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTRTFKSMPNPEGRYTFGASCVTACPYNYLSTDVGSCTLVCPAANQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPAADQCVACAHYKDPPACVARCPSGVKPDLSYMPIWAFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIMVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEALVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPELGLDVPV

SEQ ID NO: 2 - DNA, 3441 bp, Artificial sequence
  Synthetic Her2 v1 nucleotide sequence
atggaactggctgctctgtgtagatggggactgctgcttgctctgttgcctcctggagctgcttctacccaagtgtgcacaggcaccgacatgaagctgagactgcctgcttctcctgagacacacctggacatgctgagacacctgtaccagggatgtcaggtggtgcagggaaatctggaactgacctacctgcctaccaacgccagcctgagctttctgcaggacatccaagaggtgcagggatacgtgctgatcgctcacaatcaagtgagacaggtgccactgcagaggctgagaatcgttagaggcacccagctgttcgaggacaactatgctctggctgtgctggacaatggcgaccctctgaacaacaccacacctgtgacaggagcttctcctggtggactgagagaactgcagctgagaagcctgaccgagatcctgaaaggaggagtgctgatccagcggaaccctcagctgtgctaccaggacaccatcctgtggaaggacatcttccacaagaacaaccagctggctctgacactgatcgacaccaacagaagcagagcctgccatccttgctctcccatgtgcaagggctctagatgttggggagagagcagcgaggattgccagagcctgaccagaacagtgtgtgctggaggatgtgccagatgcaaaggacctctgcctaccgactgctgccacgagcaatgtgcagctggatgtacaggaccaaagcactctgattgcctggcctgcctgcacttcaaccactctggaatctgcgagctgcactgtcctgctctggtcacctacaacacacggaccttcaagagcatgcctaatcctgaaggcagatacacctttggagccagctgtgtgacagcctgtccttacaactacctgagcaccgacgtgggcagctgcacactcgtttgtcctgctgccaatcaagaagtgacagccgaggacggcacccagagatgcgagaagtgtagcaagccttgcgctagagtgtgttacggactcggcatggaacacctgagagaagtgagagccgtgaccagtgccaacatccaagagtttgctggctgcaagaagatctttggcagcctcgccttcctgcctgagagcttcgatggcgatcctgccagcaatactgctcctctgcagcctgaacagctccaggtgttcgagacactggaagagatcacaggctacctgtacatcagcgcatggccagacagcctgcctgacctgtccgtgttccagaacctgcaagtgatcagaggcagaatcctgcacaacggagcctattctctgaccctgcaaggcctgggaatcagctggctgggactgagatccctgagagagcttggatctggcctggctctgatccaccacaatacccacctgtgcttcgtgcacaccgtgccttgggaccagctgtttcggaatcctcatcaggctctgctgcacacagccaacagacctgaggatgagtgtgttggcgaaggcctggcttgtcaccagctctgtgctagaggacactgttggggacctggacctacacagtgtgtgaactgtagccagttcctgagaggccaagaatgcgtggaagagtgtagagttctgcagggactgcctcgcgagtacgtgaacgctagacactgtctgccttgtcatcccgagtgccagcctcagaatggcagcgtgacatgttttggaccagctgccgatcagtgcgtggcctgtgctcactataaggaccctccagcctgcgtggccagatgtcctagcggagtgaagcctgacctgagctacatgcccatctgggcatttccagatgaggaaggagcttgccagccttgtcctatcaactgcacccacagctgcgtggacctggacgataagggatgtccagccgagcagagagcctctccactgacctctatcatctctgccgtcgtgggcatcctgctggtggtggttctgggagttgtgttcggcatcctgatcaagagacggcagcagaagatccggaagtacaccatgcggagactgctgcaagagactgagctggtggaacctctgacacccagcggagctatgcctaaccaggctcagatgcggattctgaaagaaaccgagctgcggaaagtgaaggtgctcggctctggagcctttggcacagtgtacaaaggcatctggatccctgacggagagaacgtgaagattcctgtggccatcatggtgctgagagagaacacaagtcccaaggccaacaaagagatcctggacgaggcctacgtgatggctggtgttggcagcccttatgtgtctagactgctgggcatctgtctgaccagcaccgtgcagctggtcactcagctgatgccttacggctgcctgctggatcacgtgagagagaatagaggcagactgggctctcaggacctgctgaactggtgcatgcagatcgccaagggcatgagctacctcgaggatgtgagactggtccacagagatctggctgccagaaacgtgctcgtgaagtctcctaaccacgtgaagatcaccgacttcggactggctaggctgctggatatcgacgagacagagtaccacgctgatggaggcaaggtgcccatcaagtggatggctctggaatccatcctgagacggagattcacccaccagtccgatgtgtggtcttacggagtgacagtgtgggagctgatgaccttcggagccaagccttacgacggcatccctgccagagagatcccagatctgctggaaaagggagagagactgcctcagcctcctatctgcaccatcgacgtgtacatgattatggtcaagtgttggatgatcgacagcgagtgcagacccagattcagagaactggtgtccgagttctctcggatggccagagatcctcagagattcgtggtcatccagaacgaggatctgggacctgccagccctctggacagcaccttctacagatccctgctggaagatgacgacatgggtgacctggtggacgctgaagaagctctggttcctcagcagggcttcttctgccctgatcctgctccaggagcaggtggaatggtgcatcacagacacagaagctccagcaccagaagcggaggcggagatctgacactgggactcgagccatctgaggaagaggctcctagatctcctctggctccttctgaaggagctggaagcgacgttttcgacggagatcttggaatgggagctgccaaaggactccagtctctgcccacacacgacccatctccactgcagagatacagcgaggaccctaccgtgcctctgccaagcgagacagatggatatgtggcacctctgacctgctctcctcagccagaactgggacttgatgtgcctgtttgatga

SEQ ID NO: 3 - protein, 427 aa, Artificial sequence
  Synthetic Brachyury amino acid sequence
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 4 - DNA, 1287 bp, Artificial sequence
  Synthetic Brachyury nucleotide sequence
atgtctagccctggcacagagtctgctggcaagagcctccagtacagagtggaccatctgctgagcgctgtggagaatgaactgcaggctggaagcgagaagggagatcctacagaaagagagctgagagtcggactggaagagtccgagctgtggctgcggttcaaagaactgaccaacgagatgatcgtgaccaagaacggcagacggatgttccctgtgctgaaagtgaacgtgtccggactggaccctaacgccatgtacagctttctgctggatttcgtggcagctgacaaccacagatggaagtacgtgaacggagagtgggtgccaggaggaaaacctgaacctcaggctcctagctgcgtgtacattcaccctgacagccctaacttcggagcccactggatgaaggctcctgtgtccttcagcaaagtgaagctgaccaacaagctgaacggaggaggccagatcatgctgaacagcctgcacaagtatgagcctaggatccacatcgtcagagttggaggccctcagcggatgatcaccagccactgtttccctgagacacagttcatcgcagtgaccgcttaccagaacgaggaaatcacagccctgaagatcaagtacaatcccttcgccaaggccttcctggacgccaaagagcggagcgaccacaaagaaatgatggaagaacctggcgacagccagcagcctggctattctcaatggggatggctgctgccaggcacctccacattgtgccctccagccaatcctcatcctcagtttggcggagccctgagcctgcctagcacacacagctgcgacagataccctacactgagaagccactacgctcacagaaacaacagccctacctacagcgacaatagccctgcctgtctgagcatgctgcagtcccacgacaattggtccagcctgggaatgcctgctcacccttctatgctgcctgtctctcacaacgcctctccacctacaagcagctctcagtaccctagcctttggagcgtgtccaatggagctgtgacacctggatctcaggctgccgctgtgtctaatggactgggagcccagttcttcagaggcagccctgctcactacacacctctgacacatccagtgtctgctcctagcagcagcggaagccctctctatgaaggagccgctgcagccaccgacatcgtggattctcagtatgatgctgccgcacagggcagactgatcgcctcttggacacctgtgagcccaccttccatgtgatga

SEQ ID NO: 5 - protein, 435 aa, Homo sapiens
  Brachyury protein Isoform 1 from GenBank Accession No. O15178.1
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 6 - DNA, 1308 bp, Homo sapiens
  Coding sequence for Brachyury protein Isoform 1 GenBank Accession No. O15178.1
atgagcagccctggcacagagagcgccggcaagagcctgcagtaccgggtggaccatctgctgagcgccgtggagaatgagctgcaggccggctccgagaagggcgaccccaccgagagggaactgagagtgggcctggaagagtccgagctgtggctgcggttcaaagaactgaccaacgagatgatcgtgaccaagaacggcagacggatgttccccgtgctgaaagtgaacgtgtccggcctggaccccaacgccatgtacagctttctgctggacttcgtggccgccgacaaccacaggtggaaatacgtgaacggcgagtgggtgccaggcggcaaacctgagcctcaggcccccagctgcgtgtacatccaccccgacagccccaatttcggcgcccactggatgaaggcccccgtgtccttcagcaaagtgaagctgaccaacaagctgaacggcggaggccagatcatgctgaacagcctgcacaagtacgagccccggatccacattgtgcgcgtgggcggaccccagagaatgatcaccagccactgcttccccgagacacagtttatcgccgtgaccgcctaccagaacgaggaaatcaccgccctgaagatcaagtacaaccccttcgccaaggccttcctggacgccaaagagcggagcgaccacaaagaaatgatggaagaacccggcgacagccagcagcctggctacagccagtggggctggctgctgccaggcacctccactctgtgcccccctgccaaccctcaccctcagttcggcggagccctgagcctgcctagcacacacagctgcgacagataccccaccctgcggagccacagaagcagcccctaccccagcccatacgcccaccggaacaacagccccacctacagcgacaactcccccgcctgcctgagcatgctgcagagccacgacaactggtccagcctgggcatgcctgcccaccctagcatgctgcccgtgtcccacaatgccagcccccctaccagcagctcccagtaccctagcctgtggagcgtgtccaatggcgccgtgacacctggatctcaggccgctgccgtgagcaatggcctgggagcccagttctttagaggcagccctgcccactacacccctctgacccaccctgtgtccgcccctagctccagcggcagccctctgtatgaaggcgccgctgcagccaccgatatcgtggacagccagtacgatgccgccgctcagggcagactgatcgccagctggacccccgtgtctccccccagcatgtga

SEQ ID NO: 7 - protein, 435 aa, Artificial sequence
  Brachyury protein Isoform 1 (L254V)
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTVCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 8 - DNA, 1308 bp, Artificial sequence
  Coding sequence encoding Brachyury protein Isoform 1 with L254V
atgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaggctttccttgatgcaaaggaaagaagtgatcacaaagagatgatggaggaacccggagacagccagcaacctgggtactcccaatgggggtggcttcttcctggaaccagcaccgtttgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtga

SEQ ID NO: 9 - protein, 449 aa, Artificial sequence
  Brachyury-I3 fusion protein
MKNNLYEEKMNMSKKSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTVCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 10 - DNA, 1350 bp, Artificial sequence
  Coding sequence encoding I3 Brachyury fusion protein of SEQ ID NO 9
atgaaaaataacttgtatgaagaaaaaatgaacatgagtaagaaaagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaggctttccttgatgcaaaggaaagaagtgatcacaaagagatgatggaggaacccggagacagccagcaacctgggtactcccaatgggggtggcttcttcctggaaccagcaccgtttgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtga

SEQ ID NO: 11 - protein, 261 aa, Homo sapiens
  hCD40L from NCBI RefSeq NP_000065.1.
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 12 - DNA, 789 bp, Homo sapiens
  hCD40L from NCBI RefSeq NP_000065.1.
atgatcgagacatacaaccagacaagccctagaagcgccgccacaggactgcctatcagcatgaagatcttcatgtacctgctgaccgtgttcctgatcacccagatgatcggcagcgccctgtttgccgtgtacctgcacagacggctggacaagatcgaggacgagagaaacctgcacgaggacttcgtgttcatgaagaccatccagcggtgcaacaccggcgagagaagtctgagcctgctgaactgcgaggaaatcaagagccagttcgagggcttcgtgaaggacatcatgctgaacaaagaggaaacgaagaaagagaactccttcgagatgcagaagggcgaccagaatcctcagatcgccgctcacgtgatcagcgaggccagcagcaagacaacaagcgtgctgcagtgggccgagaagggctactacaccatgagcaacaacctggtcaccctggagaacggcaagcagctgacagtgaagcggcagggcctgtactacatctacgcccaagtgaccttctgcagcaacagagaggccagctctcaggctcctttcatcgccagcctgtgcctgaagtctcctggcagattcgagcggattctgctgagagccgccaacacacacagcagcgccaaaccttgtggccagcagtctattcacctcggcggagtgtttgagctgcagcctggcgcaagcgtgttcgtgaatgtgacagaccctagccaggtgtcccacggcaccggctttacatctttcggactgctgaagctgtgatga

SEQ ID NO: 13 - protein, 1145 aa, Artificial sequence
  Synthetic Her2 v2 amino acid sequence
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELACPALVTYNTRTAKSMPNPEGRYTFGASCVTACPYNYLSTDAGACTLVCPAANQEVTAEDGTQRCEACSKACARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPAADQCVACAHYKDPPACVARCPSGVKPDLSYMPIWAFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIMVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEALVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPELGLDVPV

SEQ ID NO: 14 - DNA, 3441 bp, Artificial sequence
  Synthetic Her2 v2 nucleotide sequence
atggaactggctgctctgtgtagatggggactgctgcttgctctgttgcctcctggagctgcttctacccaagtgtgcacaggcaccgacatgaagctgagactgcctgcttctcctgagacacacctggacatgctgagacacctgtaccagggatgtcaggtggtgcagggaaatctggaactgacctacctgcctaccaacgccagcctgagctttctgcaggacatccaagaggtgcagggatacgtgctgatcgctcacaatcaagtgagacaggtgccactgcagaggctgagaatcgttagaggcacccagctgttcgaggacaactatgctctggctgtgctggacaatggcgaccctctgaacaacaccacacctgtgacaggagcttctcctggtggactgagagaactgcagctgagaagcctgaccgagatcctgaaaggaggagtgctgatccagcggaaccctcagctgtgctaccaggacaccatcctgtggaaggacatcttccacaagaacaaccagctggctctgacactgatcgacaccaacagaagcagagcctgccatccttgctctcccatgtgcaagggctctagatgttggggagagagcagcgaggattgccagagcctgaccagaacagtgtgtgctggaggatgtgccagatgcaaaggacctctgcctaccgactgctgccacgagcaatgtgcagctggatgtacaggaccaaagcactctgattgcctggcctgcctgcacttcaaccactctggaatctgcgagctcgcctgtcctgctctggtcacctacaacacacggaccgccaagagcatgcctaatcctgaaggcagatacacctttggagccagctgtgtgacagcctgtccttacaactacctgagcaccgacgctggagcctgcacactcgtttgtcctgctgccaatcaagaagtgacggccgaggacggcacccagagatgcgaggcctgtagcaaggcttgcgctagagtgtgttacggactcggcatggaacacctgagagaagtgagagccgtgaccagtgccaacatccaagagtttgctggctgcaagaagatctttggcagcctcgccttcctgcctgagagcttcgatggcgatcctgccagcaatactgctcctctgcagcctgaacagctccaggtgttcgagacactggaagagatcacaggctacctgtacatcagcgcatggccagacagcctgcctgacctgtccgtgttccagaacctgcaagtgatcagaggcagaatcctgcacaacggagcctattctctgaccctgcaaggcctgggaatcagctggctgggactgagatccctgagagagcttggatctggcctggctctgatccaccacaatacccacctgtgcttcgtgcacaccgtgccttgggaccagctgtttcggaatcctcatcaggctctgctgcacacagccaacagacctgaggatgagtgtgttggcgaaggcctggcttgtcaccagctctgtgctagaggacactgttggggacctggacctacacagtgtgtgaactgtagccagttcctgagaggccaagaatgcgtggaagagtgtagagttctgcagggactgcctcgcgagtacgtgaacgctagacactgtctgccttgtcatcccgagtgccagcctcagaatggcagcgtgacatgttttggaccagctgccgatcagtgcgtggcctgtgctcactataaggaccctccagcctgcgtggccagatgtcctagcggagtgaagcctgacctgagctacatgcccatctgggcatttccagatgaggaaggagcttgccagccttgtcctatcaactgcacccacagctgcgtggacctggacgataagggatgtccagccgagcagagagcctctccactgacctctatcatctctgccgtcgtgggcatcctgctggtggtggttctgggagttgtgttcggcatcctgatcaagagacggcagcagaagatccggaagtacaccatgcggagactgctgcaagagactgagctggtggaacctctgacacccagcggagctatgcctaaccaggctcagatgcggattctgaaagaaaccgagctgcggaaagtgaaggtgctcggctctggagcctttggcacagtgtacaaaggcatctggatccctgacggagagaacgtgaagattcctgtggccatcatggtgctgagagagaacacaagtcccaaggccaacaaagagatcctggacgaggcctacgtgatggctggtgttggcagcccttatgtgtctagactgctgggcatctgtctgaccagcaccgtgcagctggtcactcagctgatgccttacggctgcctgctggatcacgtgagagagaatagaggcagactgggctctcaggacctgctgaactggtgcatgcagatcgccaagggcatgagctacctcgaggatgtgagactggtccacagagatctggctgccagaaacgtgctcgtgaagtctcctaaccacgtgaagatcaccgacttcggactggctaggctgctggatatcgacgagacagagtaccacgctgatggaggcaaggtgcccatcaagtggatggctctggaatccatcctgagacggagattcacccaccagtccgatgtgtggtcttacggagtgacagtgtgggagctgatgaccttcggagccaagccttacgacggcatccctgccagagagatcccagatctgctggaaaagggagagagactgcctcagcctcctatctgcaccatcgacgtgtacatgattatggtcaagtgttggatgatcgacagcgagtgcagacccagattcagagaactggtgtccgagttctctcggatggccagagatcctcagagattcgtggtcatccagaacgaggatctgggacctgccagccctctggacagcaccttctacagatccctgctggaagatgacgacatgggtgacctggtggacgctgaagaagctctggttcctcagcagggcttcttctgccctgatcctgctccaggagcaggtggaatggtgcatcacagacacagaagctccagcaccagaagcggaggcggagatctgacactgggactcgagccatctgaggaagaggctcctagatctcctctggctccttctgaaggagctggaagcgacgttttcgacggagatcttggaatgggagctgccaaaggactccagtctctgcccacacacgacccatctccactgcagagatacagcgaggaccctaccgtgcctctgccaagcgagacagatggatatgtggcacctctgacctgctctcctcagccagaactgggacttgatgtgcctgtttgatga

SEQ ID NO: 15 - protein, 205 aa, Artificial sequence
  Synthetic Twist amino acid sequence
MQDVSSSPVSPADDSLSNSEEEPDRQQPASGKRGARKRRSSRRSAGGSAGPGGATGGGIGGGDEPGSPAQGKRGKKSAGGGGGGGAGGGGGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 16 - DNA, 618 bp, Artificial sequence
  Synthetic Twist nucleotide sequence
atgcaggacgtgtccagcagccctgtgtctcctgccgacgacagcctgagcaacagcgaggaagaacccgacagacagcagcccgcctctggcaagagaggcgccagaaagagaagaagctccagaagaagcgctggcggctctgctggacctggcggagctacaggcggaggaattggaggcggagatgagcctggctctccagcccagggcaagaggggcaagaaatctgctggcggaggcggcggaggaggagctggaggcggaggaggaggcggcggaggatcaagttctggcggaggaagccctcagagctacgaggaactgcagacccagcgcgtgatggccaacgtgcgcgagagacagagaacccagagcctgaacgaggccttcgccgccctgagaaagatcatccccaccctgcccagcgacaagctgagcaagatccagaccctgaagctggccgccagatatatcgacttcctgtatcaagtgctgcagagcgacgagctggacagcaagatggccagctgctcctacgtggcccacgagagactgagctacgccttcagcgtgtggcggatggaaggcgcctggtctatgagcgccagccactga

SEQ ID NO: 17 - protein, 260 aa, Artificial Sequence
  Synthetic murine CD40L amino acid sequence
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 18 - DNA, 786 bp, Artificial sequence
  Synthetic murine CD40L nucleotide sequence
atgatcgagacatacagccagcccagccccagaagcgtggccacaggactgcctgccagcatgaagatctttatgtacctgctgaccgtgttcctgatcacccagatgatcggcagcgtgctgttcgccgtgtacctgcacagacggctggacaaggtggaagaggaagtgaacctgcacgaggacttcgtgttcatcaagaaactgaagcggtgcaacaagggcgagggcagcctgagcctgctgaactgcgaggaaatgagaaggcagttcgaggacctcgtgaaggacatcaccctgaacaaagaggaaaagaaagaaaactccttcgagatgcagaggggcgacgaggaccctcagatcgctgctcacgtggtgtccgaggccaacagcaacgccgcttctgtgctgcagtgggccaagaaaggctactacaccatgaagtccaacctcgtgatgctggaaaacggcaagcagctgacagtgaagcgcgagggcctgtactatgtgtacacccaagtgacattctgcagcaacagagagcccagcagccagaggcccttcatcgtgggactgtggctgaagcctagcagcggcagcgagagaatcctgctgaaggccgccaacacccacagcagctctcagctgtgcgagcagcagagcgtgcacctgggcggagtgttcgagctgcaagctggcgcctccgtgttcgtgaacgtgacagaggccagccaagtgatccacagagtgggcttcagcagctttggactgctgaaactgtaatga

(19) United States
(12) Patent Application Publication
(10) Pub. No.: US 2024/0269254 A1
(43) Pub. Date: Aug. 15, 2024

(54) Combination Therapy for Treating Cancer with an Intravenous Administration of a Recombinant MVA and an Immune Checkpoint Antagonist or Agonist

(71) Applicant: Bavarian Nordic A/S, Hellerup (DK)

(72) Inventors: Henning Lauterbach, Eching (DE); Jose Medina Echeverz, Munich (DE); Maria Hinterberger, Sauerlach (DE)

(73) Assignee: Bavarian Nordic A/S, Hellerup (DK)

(21) Appl. No.: 17/282,774

(22) PCT Filed: Oct. 4, 2019
(86) PCT No.: PCT/EP19/76947

§ 371 Date: Apr. 4, 2021

(30) Foreign Application Priority Data
Oct. 5, 2018 (EP) 18199002.9

(51) Int. Cl. A61K 39/00; A61P 35/00

(52) U.S. Cl. CPC A61K 39/00119; A61K 39/001106; A61P 35/00; A61K 2039/5254; A61K 2039/5256; A61K 2039/545; A61K 2039/55516; C12N 2710/24134; C12N 2710/24171

(57) ABSTRACT

The invention relates to a pharmaceutical combination and related methods for reducing tumor volume and/or increasing the survival of a cancer patient. The combination comprises an intravenous administration of a recombinant MVA encoding CD40L and an administration of an antagonist or agonist of an immune checkpoint molecule.

### FIELD OF THE INVENTION

The present invention relates to a combination therapy for the treatment of cancers, the treatment includes an intravenously administered recombinant modified vaccinia Ankara (MVA) virus comprising a nucleic acid encoding CD40L in combination with an antagonist or agonist of an immune checkpoint molecule.

### BACKGROUND OF THE INVENTION

Recombinant poxviruses have been used as immunotherapy vaccines against infectious organisms and, more recently, against tumors (Mastrangelo et al. (2000) J Clin Invest. 105(8):1031-1034).

One poxviral strain that has proven useful as an immunotherapy vaccine against infectious disease and cancer is the Modified Vaccinia Ankara (MVA) virus. MVA was generated by 516 serial passages on chicken embryo fibroblasts of the Ankara strain of vaccinia virus (CVA) (for review see Mayr et al. (1975) Infection 3: 6-14). As a consequence of these long-term passages, the genome of the resulting MVA virus had about 31 kilobases of its genomic sequence deleted and, therefore, was described as highly host cell restricted for replication to avian cells (Meyer et al. (1991) J. Gen. Virol. 72: 1031-1038). It was shown in a variety of animal models that the resulting MVA was significantly avirulent (Mayr & Danner (1978) Dev. Biol. Stand. 41: 225-34). Strains of MVA having enhanced safety profiles for the development of safer products, such as vaccines or pharmaceuticals, have been described. (See International PCT publication WO2002042480; see also, e.g., U.S. Pat. Nos. 6,761,893 and 6,913,752, all of which are incorporated by reference herein). Such variants are capable of reproductive replication in non-human cells and cell lines, especially in chicken embryo fibroblasts (CEF), but are replication incompetent in human cell lines, in particular including HeLa, HaCat and 143B cell lines. Such strains are also not capable of reproductive replication in vivo, for example, in certain mouse strains, such as the transgenic mouse model AGR 129, which is severely immune-compromised and highly susceptible to a replicating virus (see U.S. Pat. No. 6,761,893). Such MVA variants and its derivatives, including recombinants, referred to as “MVA-BN,” have been described (see International PCT publication WO2002/042480; see also, e.g., U.S. Pat. Nos. 6,761,893 and 6,913,752).

The use of poxviral vectors that encode tumor-associated antigens (TAAs) have been shown to successfully reduce tumor size as well as increase overall survival rate of cancer patients (see, e.g., WO 2014/062778). It has been demonstrated that when a cancer patient is administered a poxviral vector encoding a TAA, such as HER2, CEA, MUC1, and/or Brachyury, a robust and specific T-cell response is generated by the patient to fight the cancer (Id.; see also, Guardino et al. ((2009) Cancer Res. 69 (24), doi 10.1158/0008-5472.SABCS-09-5089), Heery et al. (2015) JAMA Oncol. 1: 1087-95).

In addition to their effectiveness with TAAs, poxviruses, such as MVA have been shown to have enhanced efficacy when combined with a CD40 agonist such as CD40 Ligand (CD40L) (see WO 2014/037124). CD40/CD40L is a member of the tumor necrosis factor receptor/tumor necrosis factor (“TNFR/TNF”) superfamily. While CD40 is constitutively expressed on many cell types, including B-cells, macrophages and DCs, its ligand CD40L is predominantly expressed on activated CD4+ T-cells (see Lee et al. (2002) J. Immunol. 171(11): 5707-5717; Ma and Clark (2009) Semin. Immunol. 21(5): 265-272). The cognate interaction between DCs and CD4+ T-cells early after infection or immunization ‘licenses’ DCs to prime CD8+ T-cell responses (Ridge et al. (1998) Nature 393(6684): 474-478). DC licensing results in the upregulation of co-stimulatory molecules, increased survival and better cross-presenting capabilities of DCs. This process is mainly mediated via CD40/CD40L interaction (Bennet et al. (1998) Nature 393(6684): 478-480; Schoenberger et al. (1998) Nature 393(6684): 480-483), but CD40/CD40L-independent mechanisms also exist (CD70, LT.β.R). Interestingly, a direct interaction between CD40L expressed on DCs and CD40 expressed on CD8+ T-cells has also been suggested, providing a possible explanation for the generation of helper-independent CTL responses (Johnson et al. (2009) Immunity 30(2): 218-227).

Several studies indicate that agonistic anti-CD40 antibodies may be useful as a vaccine adjuvant. In addition, recombinant adenovirus (Kato et al. (1998) J. Clin. Invest. 101(5):1133-1141) and vaccinia virus (Bereta et al. (2004) Cancer Gen. Ther. 11(12): 808-818) encoding CD40L have been created that showed superior immunogenicity in vitro and in vivo compared to non-adjuvanted viruses.

CD40L, when encoded as part of an MVA, was shown to be able to induce and enhance the overall T-cell response for a disease associated antigen (WO 2014/037124). In WO 2014/037124 it was shown that a recombinant MVA encoding CD40L and a heterologous antigen was able to enhance DC activation in vivo, increase T-cell responses specific to the heterologous antigen and enhance the quality and quantity of CD8 T-cells (Id.).

The use of checkpoint inhibitors, or antagonists or agonists of immune checkpoints molecules, for cancer therapy has also seen considerable success in the past several years. Inhibitory receptors on immune cells are pivotal regulators of immune escape in cancer (Woo et al. (2011) Cancer Res. 72(4): 917-27). Among these inhibitory receptors, CTLA-4 (Cytotoxic T-Lymphocyte-Associated protein 4) serves as a dominant off-switch while other receptors such as PD-1 (programmed death 1, CD279) and LAG-3 (lymphocyte activation gene, CD223) seem to serve more subtle rheostat functions (Id).

CTLA-4 is an immune checkpoint molecule, which is up-regulated on activated T-cells (Mackiewicz (2012) Wspolczesna Onkol 16 (5):363-370). An anti-CTLA4 mAb can block the interaction of CTLA-4 with CD80/86 and switch off the mechanism of immune suppression and enable continuous stimulation of T-cells by DCs. Two IgG monoclonal antibodies (mAb) directed against CTLA-4, ipilimumab and tremelimumab, have been used in clinical trials in patients with melanoma. However, treatments with anti-CTLA-4 antibodies have shown high levels of immune-related adverse events (Id).

Another human mAb modulating the immune system is BMS-936558 (MDX-1106) directed against the programmed cell death-1 receptor (PD-1), the ligand of which (PD-L1) can be directly expressed on melanoma cells (Id). PD-1 is a part of the B7:CD28 family of co-stimulatory molecules that regulate T-cell activation and tolerance, and thus PD-1 antagonists such as PD-1 antibodies can play a role in breaking tolerance (Id).

Engagement of the PD-1/PD-L1 pathway results in inhibition of T-cell effector function, cytokine secretion and proliferation (Turnis et al. (2012) OncoImmunology 1(7): 1172-1174). High levels of PD-1 are associated with exhausted or chronically stimulated T cells (Id). Moreover, increased PD-1 expression correlates with reduced survival in cancer patients (Id).

There are currently several PD-1 and PD-L1 antibodies approved for the treatment of cancers. Some of these include Nivolumab, Pembrolizumab, Atezolizumab, Avelumab and Durvalumab, while more are currently under development (Pidilizumab, AMP-224, AMP-514, PDR001, Cemiplimab, BMS-936559, and CK-3012).

Another immune checkpoint inhibitor, LAG-3, is a negative regulatory molecule expressed upon activation of various lymphoid cell types (Id). LAG-3 is required for the optimal function of both natural and induced immunosuppressive Treg cells (Id).

Combinatorial blockade of PD-1 and LAG-3 with monoclonal antibodies synergistically limited the growth of established tumors (Woo et al. (2011) Cancer Res. 72(4): 917-27). Although anti-LAG-3/anti-PD-1 combinatorial immunotherapy effectively cleared established Sa1N and MC38 tumors, this therapy was not effective against established B16 tumors (Id). Turnis et al. reported that their study “highlighted the difficulty in predicting the outcome of combination treatments” (Turnis et al. (2012) OncoImmunology 1(7): 1172-1174).

The inducible co-stimulatory molecule (ICOS) has been reported to be highly expressed on Tregs infiltrating various tumors, including melanoma and ovarian cancers (Faget et al. (2013) OncoImmunology 2:3, e23185). It has also been reported that the ICOS/ICOSL interaction occurs during the interaction of tumor-associated (TA)-Tregs with TA-pDCs in breast carcinoma (Id). Antagonist antibodies against ICOS have been used to inhibit ICOS/ICOS-L interaction and abrogate proliferation of Treg induced by pDC (see WO 2012/131004). An antagonist antibody was used in a murine model of mammary tumor to reduce tumor progression (Id).

An agonist antibody directed against ICOS has been suggested as being useful in combination with a blocking anti-CTLA-4 antibody and a blocking anti-PD-1 antibody for the treatment of tumors (see WO 2011/041613).

There is clearly a substantial unmet medical need for additional cancer treatments, including active immunotherapies and cancer vaccines. In many aspects, the embodiments of the present disclosure address these needs by providing combination therapies that increase and improve the cancer treatments currently available.

### BRIEF SUMMARY OF THE INVENTION

It was determined in the various embodiments of the present invention that a recombinant MVA encoding a CD40L antigen, when administered intravenously to a patient in combination with an administration of an immune checkpoint antagonist or agonist enhances treatment of a cancer patient, more particularly increases reduction in tumor volume and/or increases survival of the cancer patient.

Accordingly, in one embodiment, the present invention includes a combination for use in reducing tumor size and/or increasing survival in a cancer patient, the combination comprising: a) a recombinant modified vaccinia virus Ankara (MVA) comprising a first nucleic acid encoding a tumor-associated antigen (TAA) and a second nucleic acid encoding CD40L that when administered intravenously induces both an enhanced Natural Killer (NK) cell response and an enhanced T cell response in the cancer patient as compared to a NK cell and T cell response induced by a non-intravenous administration of a recombinant MVA comprising a first nucleic acid encoding a TAA and a second nucleic acid encoding CD40L; and b) at least one antagonist or agonist of an immune checkpoint molecule; wherein administration of a) and b) to the cancer patient reduces tumor size and/or increases the survival rate of the cancer patient as compared to a non-intravenous administration of either a) or b) alone.

In an additional embodiment, there is a method for reducing tumor size and/or increasing survival in a cancer patient, the method comprising: a) administering to the cancer patient a recombinant modified Vaccinia Ankara (MVA) virus comprising a first nucleic acid encoding a tumor-associated antigen (TAA) and a second nucleic acid encoding CD40L, that when administered intravenously induces both an enhanced Natural Killer (NK) cell response and an enhanced T cell response as compared to an NK cell response and a T cell response induced by a non-intravenous administration of a recombinant MVA virus comprising a first nucleic acid encoding a TAA and a second nucleic acid encoding CD40L; and b) administering to the cancer patient at least one antagonist or agonist of an immune checkpoint molecule; wherein (a) and (b) are to be administered as a combination treatment; and wherein administration of a) and b) to the cancer patient reduces tumor size and/or increases the survival rate of the cancer patient as compared to a non-IV administration of a) or an administration of b) alone.

In preferred embodiments, the at least one antagonist or agonist of an immune checkpoint molecule comprises a CTLA-4 antagonist, a PD-1 antagonist, a −PD-L1 antagonist, a LAG-3 antagonist, a TIM-3 antagonist, or an ICOS agonist. In more preferred embodiments, the at least one antagonist or agonist of an immune checkpoint molecule comprises a CTLA-4 antagonist, a PD-1 antagonist, or a PD-L1 antagonist.

In still more embodiments, the at least one of antagonist or agonist of an immune checkpoint molecule comprises an antibody able to block the function of the immune checkpoint molecule. In preferred embodiments, the antibody is selected from a CTLA-4 antibody, a PD-1 antibody, a PD-L1 antibody, a LAG-3 antibody, an ICOS antibody, and a TIM-3 antibody, respectively. In more preferred embodiments, the at least one antagonist or agonist comprises a CTLA-4, a PD-1, or a PD-L1 antibody.

In still additional embodiments, the first nucleic acid encoding the TAA is selected from the group consisting of: carcinoembryonic antigen (CEA), Mucin 1, cell surface associated (MUC-1), Prostatic Acid Phosphatase (PAP), Prostate Specific Antigen (PSA), human epidermal growth factor receptor 2 (HER2), survivin, tyrosine related protein 1 (TRP1), tyrosine related protein 2 (TRP2), Brachyury antigen, or combinations thereof.

In one or more preferred embodiments, the recombinant MVA is MVA-BN or a derivative thereof.

Additional objects and advantages of the invention will be set forth in part in the description which follows, and in part will be obvious from the description or may be learned by practice of the invention. The objects and advantages of the invention will be realized and attained by means of the elements and combinations particularly pointed out in the appended claims.

The accompanying drawings, which are incorporated in and constitute a part of this specification, illustrate one or more embodiments of the invention and together with the description, serve to explain the principles of the invention.

### BRIEF DESCRIPTION OF THE DRAWINGS

FIGS. 1A-1G show that intravenous (IV) administration of MVA-OVA (rMVA) leads to a stronger systemic activation of NK cells as compared to subcutaneous (SC) administration. NK cell activation is further enhanced when the MVA encodes CD40L (rMVA-CD40L). Shown are the results of Example 1, wherein staining to assess NK cell frequencies and expression (shown as Geometric Mean Fluorescence Intensity (GMFI)) of the named protein markers in NKp46⁺CD3⁻ cells was assessed in the spleen. FIG. 1A) NKp46⁺ CD3⁻ cells;—FIG. 1B) CD69; FIG. 1C) NKG2D; FIG. 1D) FasL; FIG. 1E); Bcl-X_{L}; FIG. 1F), CD70; and FIG. 1G) IFN-γ.

FIGS. 2A-2G show that IV administration of MVA-OVA (rMVA) leads to a stronger systemic activation of NK cells as compared to SC administration. NK cell activation is further enhanced when the MVA encodes CD40L (rMVA-CD40L). Shown are the results of Example 1, wherein staining to assess NK cell frequencies and expression (shown as Geometric Mean Fluorescence Intensity (GMFI)) of the named protein markers in NKp46⁺CD3⁻ cells was assessed in the liver. FIG. 2A) NKp46⁺CD3⁻ cells; FIG. 2B) CD69; FIG. 2C) NKG2D; FIG. 2D) FasL; FIG. 2E); Bcl-X_{L}; FIG. 2F), CD70; and FIG. 2G) IFN-γ.

FIGS. 3A-3G show that IV administration of MVA-OVA (rMVA) leads to a stronger systemic activation of NK cells as compared to SC administration. NK cell activation is further enhanced when the MVA encodes CD40L (rMVA-CD40L). Shown are the results of Example 1, wherein staining to assess NK cell frequencies and expression (shown as Geometric Mean Fluorescence Intensity (GMFI)) of the named protein markers in NKp46⁺CD3⁻ cells was assessed in the lung. FIG. 3A) NKp46⁺CD3⁻ cells; FIG. 3B) CD69; FIG. 3C) NKG2D; FIG. 3D) FasL; FIG. 3E); Bcl-X_{L}; FIG. 3F), CD70; and FIG. 3G) IFN-γ.

FIGS. 4A-4F show that intravenous (IV) administration of MVA-HER2v1-Twist-CD40L leads to a stronger systemic activation of NK cells as compared to subcutaneous (SC) administration. Shown are the results of Example 1, wherein staining to assess NK cell frequencies and expression (shown as Geometric Mean Fluorescence Intensity (GMFI)) of the named protein markers in NKp46⁺CD3⁻ cells was assessed in the spleen. FIG. 4A) NKp46⁺CD3⁻ cells; FIG. 4B) CD69; FIG. 4C) FasL; FIG. 4D); Bcl-X_{L}; FIG. 4E), CD70; and FIG. 4F) IFN-γ.

FIGS. 5A-5F show that IV administration of MVA-HER2v1-Twist-CD40L leads to a stronger systemic activation of NK cells as compared to SC administration. Shown are the results of Example 1, wherein staining to assess NK cell frequencies and expression (shown as Geometric Mean Fluorescence Intensity (GMFI)) of the named protein markers in NKp46⁺CD3⁻ cells was assessed in the liver. FIG. 5A) NKp46⁺ CD3⁻ cells; FIG. 5B) CD69; FIG. 5C) FasL; FIG. 5D); Bcl-X_{L}; FIG. 5E), CD70; and FIG. 5F) IFN-γ.

FIGS. 6A-6F show that IV administration of MVA-HER2v1-Twist-CD40L leads to a stronger systemic activation of NK cells as compared to SC administration. Shown are the results of Example 1, wherein staining to assess NK cell frequencies and expression (shown as Geometric Mean Fluorescence Intensity (GMFI)) of the named protein markers in NKp46⁺CD3⁻ cells was assessed in the lung. FIG. 6A) NKp46⁺ CD3⁻ cells; FIG. 6B) CD69; FIG. 6C) FasL; FIG. 6D); Bcl-X_{L}; FIG. 6E), CD70; and FIG. 6F) IFN-γ.

FIGS. 7A-7F show that IV administration of MVA-OVA-CD40L (rMVA-CD40L) leads to enhanced levels of IL-12p70 and IFN-γ in the serum. Shown are the results of Example 2. FIG. 7A) The concentration of IFN-γ was higher after rMVA-CD40L as compared to MVA-OVA (rMVA) immunization. FIG. 7B) The NK cell activating cytokine IL-12p70 was only detectable after MVA-CD40L immunization. High serum levels of IFN-γ are in line with higher frequencies of IFN-γ⁺ NK cells (see FIG. 1G) and CD69⁺ granzyme B⁺ NK cells in the spleen (FIG. 7C) after rMVA-CD40L immunization. Similar responses were seen in NHPs (Macaca fascicularis) after IV injection of MVA-MARV-GP-huCD40L (rMVA-CD40L), namely higher serum concentrations of IFN-γ (FIG. 7D) and IL-12p40/70 (FIG. 7E) as well as more proliferating (Ki67⁺) NK cells (FIG. 7F) as compared to MVA-MARV-GP (rMVA).

FIG. 8 shows that IV immunization induces stronger CD8 T cell responses than SC immunization. Described in Example 3, C57BL/6 mice were immunized either SC or IV with MVA-OVA on days 0 and 15. OVA-specific CD8 T cell responses in the blood were assessed after staining with H-2K^{b}/OVA₂₅₇₋₂₆₄ dextramers.

FIG. 9 shows that CD8 T cell responses can be further enhanced by MVA-CD40L. Described in Example 4, C57BL/6 mice were immunized IV with MVA-OVA (rMVA) or MVA-OVA-CD40L (rMVA-CD40L) on days 0 and 35. OVA-specific CD8 T cell responses in the blood were assessed after staining with H-2K^{b}/OVA₂₅₇₋₂₆₄ dextramers.

FIGS. 10A-10B shows repeated NK cell activation and proliferation after prime/boost immunization. Described in Example 5, C57BL/6 mice were immunized IV either with PBS, MVA-OVA (rMVA) or MVA-OVA-CD40L (rMVA-CD40L) as shown in Table 1. NK cells (NKp46⁺CD3⁻) were analyzed in the blood by flow cytometry one and four days after second and third immunization. FIG. 10A) Shows GMFI CD69 and FIG. 10B) shows frequency of Ki67⁺ NK cells.

FIGS. 11A-11M show systemic cytokine responses after prime/boost immunization. Described in Example 6, C57BL/6 mice were immunized IV either with PBS, MVA-OVA (rMVA) or MVA-OVA-CD40L (rMVA-CD40L) as shown in Table 1. Serum cytokine levels were measured at 6 hours post immunization. Shown are the results FIG. 11A) IL-6; FIG. 11B) CXCL10; FIG. 11C) IFN-α; FIG. 11D) IL-22; FIG. 11E) IFN-γ; FIG. 11F) CXCL1; FIG. 11G) CCL4; FIG. 11H) CCL7); FIG. 11I) CCL2; FIG. 11J) CCL5; FIG. 11K) TNF-α; FIG. 11L) IL-12p70; and FIG. 11M) IL-18.

FIGS. 12A-12B show CD8 and CD4 effector T cell induction after MVA and MVA-CD40L prime/boost immunization. Described in Example 7, C57BL/6 mice were immunized IV either with PBS, MVA-OVA (rMVA) or MVA-OVA-CD40L (rMVA-CD40L). Phenotypically, effector T cells were identified by the expression of CD44 and the lack of surface CD62L. FIG. 12A) CD44⁺ CD62L⁻ CD8 T cells and FIG. 12B) CD4 T cells in the blood were monitored.

FIGS. 13A-13B show superior anti-tumor effect of IV rMVA-CD40L immunization in a heterologous prime boost scheme in a melanoma model. C57BL/6 mice bearing palpable B16.OVA tumors were primed (dotted line) either with PBS, MVA-OVA (rMVA) or MVA-OVA-CD40L (rMVA-CD40L) SC or IV as described in Example 8. Mice received subsequent boosts with FPV-OVA 7 and 14 days after prime (dashed lines). Tumor growth was measured at regular intervals. Shown are FIG. 13A) tumor mean volume and FIG. 13B) survival of tumor-bearing mice by day 45 after tumor inoculation.

FIG. 14 shows efficient tumor control after a single IV immunization with MVA-OVA-CD40L (rMVA-CD40L). C57BL/6 mice bearing palpable B16.OVA tumors were primed IV or received IV prime and boost as described in Example 9. Tumor growth was measured at regular intervals. Shown is the tumor mean volume.

FIGS. 15A-15C show increased T cell infiltration in the tumor microenvironment (TME) after rMVA-CD40L immunization. C57BL/6 mice bearing palpable B16.OVA tumors were immunized IV either with PBS, MVA-OVA (rMVA) or MVA-OVA-CD40L (rMVA-CD40L) as described in Example 10. Seven days later, mice were sacrificed. FIG. 15A) Frequency of CD8⁺ T cells among CD45⁺ leukocytes in spleen, tumor-draining lymph nodes (TDLN) and tumor tissues; FIG. 15B) distribution of OVA₂₅₇₋₂₆₄-specific CD8⁺ T cells in different organs upon immunization; FIG. 15C) GMFI of PD-1 and Lag3 on tumor-infiltrating OVA₂₅₇₋₂₆₄-specific CD8⁺ T cells.

FIG. 16 show a long-term reduction of regulatory T cells (Treg) in the TME after rMVA-CD40L immunization. Purified OVA-specific TCR-transgenic CD8 T cells (OT-I) were IV transferred into B16.OVA tumor bearers when tumors were palpable as described in Example 11. When tumors reached at least 60 mm³ in volume animals were immunized IV with MVA-BNK, MVA-OVA (rMVA) or MVA-OVA-CD40L (rMVA-CD40L). 17 days later, mice were sacrificed for further analysis. Frequency of Foxp3⁺ CD4⁺ Treg among CD4⁺ T cells in tumor tissues.

FIGS. 17A-17F show persistence of TAA-specific CD8 T cells with a less exhausted phenotype in the TME after rMVA-CD40L immunization. Purified OVA-specific TCR-transgenic CD8 T cells (OT-I) were IV transferred into B16.OVA tumor bearers. When tumors reached at least 60 mm³ in volume animals were immunized IV with MVA-BN®, MVA-OVA (rMVA) or MVA-OVA-CD40L (rMVA-CD40L). 17 days later, mice were sacrificed and analyzed for: FIG. 17A) Frequency of CD8⁺ T cells among leukocytes in tumor tissues; FIG. 17B) Frequency of Lag3⁺ PD1⁺ within CD8⁺ T cells; FIG. 17C) Frequency of Eomes⁺ PD1⁺ T cells within CD8⁺ T cells; FIG. 17D) Presence of OT-I-transgenic CD8⁺ T cells within the TME upon immunization; FIG. 17E) Frequency of Lag3⁺ PD1⁺ exhausted T cells within OT-I CD8⁺ T cells; and FIG. 17F) Frequency of Eomes⁺ PD1⁺ exhausted T cells within OT-I CD8⁺T cells.

FIGS. 18A-18D show transgene expression of MVA-HER2v1-Brachyury-CD40L. HeLa cells were left untreated (Mock; filled grey line) or infected with MVA-BN (filled black line) or MVA-HER2v1-Brachyury-CD40L (open black line) as described in Example 12. Then, protein expression from FIG. 18A) MVA, FIG. 18B) HER2v1, FIG. 18C) Brachyury, and FIG. 18D) CD40L was determined by flow cytometry (see histograms).

FIGS. 19A-19D show dose dependent and enhanced activation of human DCs by MVA-HER2v1-brachyury-CD40L as compared to MVA-HER2v1-brachyury. Monocyte-derived DCs were generated after enrichment of CD14⁺ monocytes from human PBMCs and cultured for 7 days in the presence of GM-CSF and IL-4 as described in Example 14. DCs were stimulated with MVA-HER2v1-brachyury or MVA-HER2v1-brachyury-CD40L. Expression of FIG. 19A) CD40L; FIG. 19B) CD86; and FIG. 19C) MHC class II was measured by flow cytometry. FIG. 19D) The concentration of IL-12p70 was quantified.

FIG. 20 shows increased infiltration of HER2-specific CD8⁺ T cells producing IFN-γ in the tumor microenvironment upon IV MVA-HER2v1-Twist-CD40L immunization. Balb/c mice bearing palpable CT26.HER2 tumors were immunized either with PBS or MVA-HER2v1-Twist-CD40L IV as described in Example 16. Seven days later, spleen and tumor-infiltrating CD8⁺ T cells isolated by magnetic cell sorting and cultured in the presence of HER2 peptide-loaded dendritic cells for 5 hours. Graph shows percentage of CD44⁺ IFN-γ⁺ cells among CD8⁺ T cells.

FIG. 21 shows increased overall survival and tumor reduction in IV administration of rMVA-CD40L combined with anti-PD1 checkpoint blockade. C57BL/6 mice bearing 85 mm³ MC38 colon cancer were immunized IV either with MVA-AH1A5-p15e-TRP2-CD40L (shown as rMVA-p15eCD40L), or received PBS. Immunization was subsequently followed by anti PD-1 antibody administration as described in Example 17. Tumor growth was measured at regular intervals. Shown are the tumor mean volume (A) and tumor-free survival (B).

FIG. 22 shows increased overall survival and tumor reduction in IV administration of MVA-Twist-Her2-CD40L combined with anti-PD1 checkpoint blockade. C57BL/6 mice bearing 85 mm³ MC38.HER2 colon cancer were immunized IV either with MVA-Twist-Her2v1-CD40L, MVA-Twist-Her2v1-CD40L and PD-1, PD-1 alone, or received PBS. Immunization was subsequently followed by anti PD-1 antibody administration as described in Example 18. Tumor growth was measured at regular intervals. Shown are the tumor mean volume (FIG. 22A) and tumor-free survival (FIG. 22B).

FIGS. 23A, 23B, and 23C show the antitumor effect of intravenous injection of MVA virus encoding the endogenous retroviral antigen Gp70. CT26.wt tumor-bearing Balb/c mice (n=5/group) were grouped and received intravenous (i.v.) PBS or 5×10⁷ TCID₅₀ MVA, MVA-Gp70, or MVA-Gp70-CD40L at day 12 after tumor inoculation. Tumor growth was measured at regular intervals. Shown are tumor mean diameter (FIG. 23A) and tumor mean volume (FIG. 23B). Seven days after immunization, blood cells were restimulated and the percentage of CD44+ IFN-γ+ cells among CD8+ T cells in blood upon stimulation is shown (FIG. 23C).

FIGS. 24A and 24B show the antitumor effect of intravenous injection of MVA virus encoding the endogenous retroviral antigen Gp70. B16.F10 tumor-bearing C57BL/6 mice (n=5/group) were grouped and received intravenous (i.v.) PBS or 5×10⁷ TCID₅₀ MVA, MVA-Gp70 or MVA-Gp70-CD40L at day 7 after tumor inoculation. Tumor growth was measured at regular intervals. Shown are tumor mean volume (FIG. 24A) and percentage of CD44+IFN-γ+ cells among CD8+ T cells in blood upon stimulation with p15e peptide 7 days after immunization (FIG. 24B).

### DETAILED DESCRIPTION OF THE INVENTION

It is to be understood that both the foregoing Summary and the following Detailed Description are exemplary and explanatory only and are not restrictive of the invention, as claimed.

CD40L, when encoded as part of an MVA, was shown to be able to induce and enhance the overall T-cell response for a disease associated antigen. WO 2014/037124. In WO 2014/037124 it was shown that a recombinant MVA encoding CD40L and a heterologous antigen was able to enhance DC activation in vivo, increase T-cell responses specific to the heterologous antigen and enhance the quality and quantity of CD8 T-cells. Id. To induce synergistic anti-tumor responses, the various pharmaceutical combinations of the present invention were developed. In several aspects, the various combinations induce both highly effective tumor specific killer T cells and natural killer (NK) cells that are able to kill tumor cells when combined with a checkpoint antagonist or agonist. This enhanced NK cell and T cell activation when combined with the enhanced killer T cell response also induced by the MVA, is shown to synergistically increase tumor reduction and overall survival rate in cancer subjects when combined with a checkpoint antagonist or agonist.

In one embodiment, the present invention is a combination, or combination therapy, comprising: a) an intravenous (IV) administration of a recombinant MVA that comprises a first nucleic acid encoding a tumor-associated antigen (TAA) and a second nucleic acid encoding CD40L, and b) at least one antagonist or agonist of an immune checkpoint molecule As noted herein, in various embodiments, the at least one antagonist or agonist of an immune checkpoint molecule is selected from a CTLA-4 antagonist, a PD-1 antagonist, a PD-L1 antagonist, a LAG-3 antagonist, a TIM-3 antagonist, and a ICOS agonist. Also noted herein, in more preferable embodiments, the at least one antagonist or agonist of an immune checkpoint molecule comprises an antibody. Still, in more preferable embodiments, the CTLA-4 antagonist, PD-1 antagonist, PD-L1 antagonist, LAG-3 antagonist, TIM-3 antagonist, and the ICOS agonist comprise a CTLA-4 antibody, a PD-1 antibody, a PD-L1 antibody, a LAG-3 antibody, a TIM-3 antibody, and an ICOS antibody, respectively.

Described and illustrated in the present application, the combination and/or combination therapy of the present invention enhances multiple aspects of a cancer patient's immune response. In at least one aspect, the combination synergistically enhances both the innate and adaptive immune responses and, when combined with an antagonist or agonist of an immune checkpoint molecule, reduces tumor volume and increase survival of a cancer patient. One or more of the enhanced effects of the combination and or therapy are summarized as follows.

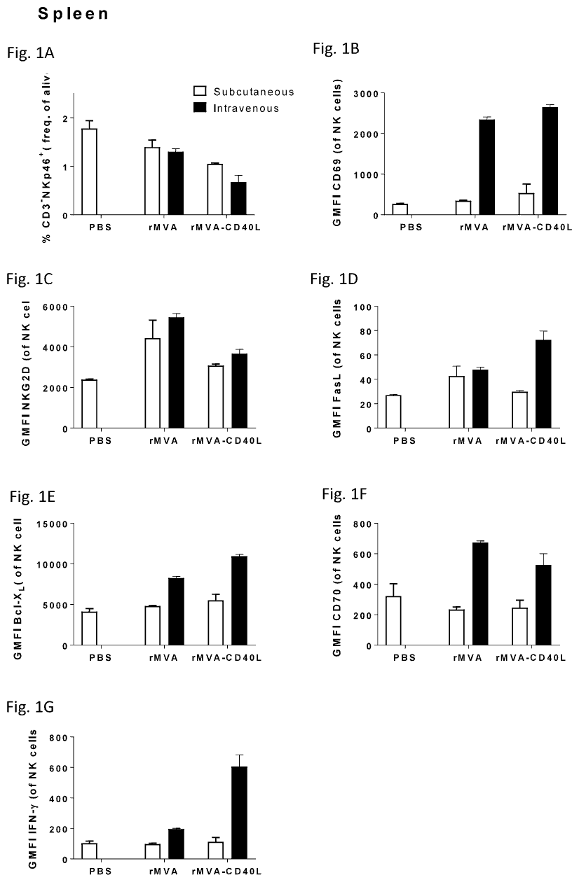

IV administration of recombinant MVA enhances NK cell response. In one aspect, the present invention includes a recombinant MVA administered intravenously to a subject, wherein the IV administration induces an enhanced innate immune response, more particularly an enhanced NK cell response in the subject as compared to a NK cell response induced by a non-IV administration of a recombinant MVA to the subject. Shown in FIGS. 1A-7F and 10A-12B, IV administration of recombinant MVA induced a robust systemic NK cell response in several compartments in both a single IV administration and when administered intravenously as a homologous prime-boost, as compared to a non-IV administration.

Illustrated in FIGS. 1A through 1F, FIGS. 2A-2G, FIGS. 3A-3G, FIGS. 4A-4F, FIGS. 5A-5F, and FIGS. 6A-6F, the quality of the NK cell response was enhanced as compared to a non-IV administration. The activation marker CD69 is increased in all organs analyzed (spleen, liver and lung). The anti-apoptotic Bcl-family member Bcl-XL, that enhances NK cell survival, co-stimulatory CD70 and the effector cytokine IFN-γ were increased both in spleen and lung. Expression of the activating Natural Killer Group 2D (NKG2D) receptor was especially enhanced in liver and lung after IV compared to SC injection. NKG2D binds to ligands on tumor cells promoting their elimination (Garcia-Cuesta et al., 2015, Reviewed in Spear et al., 2013).

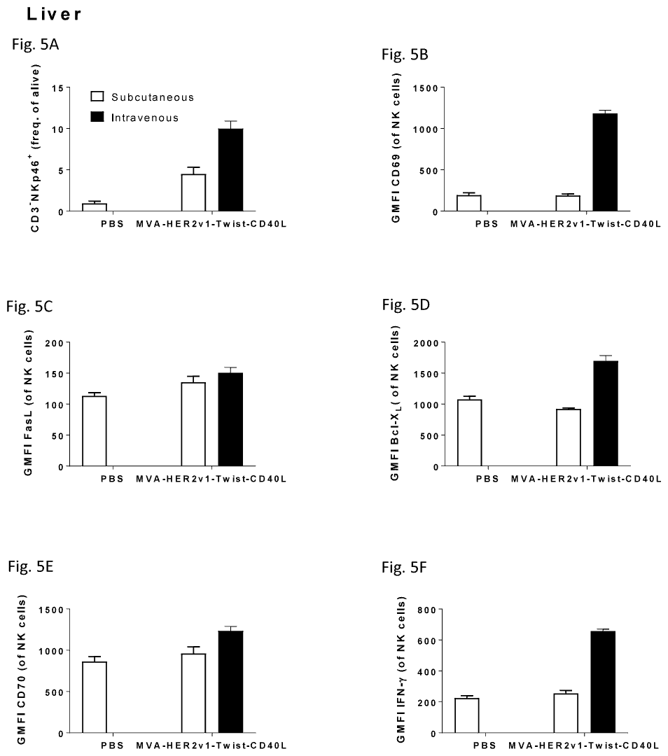

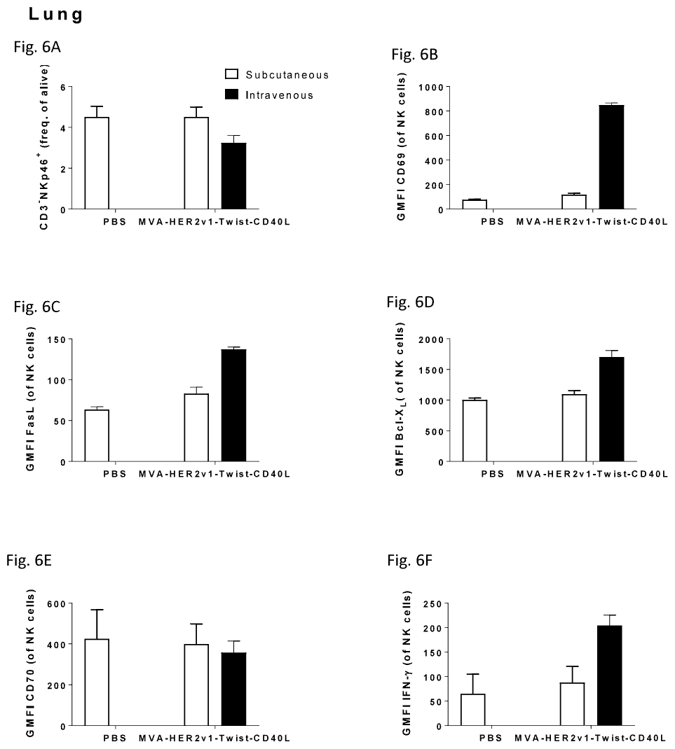

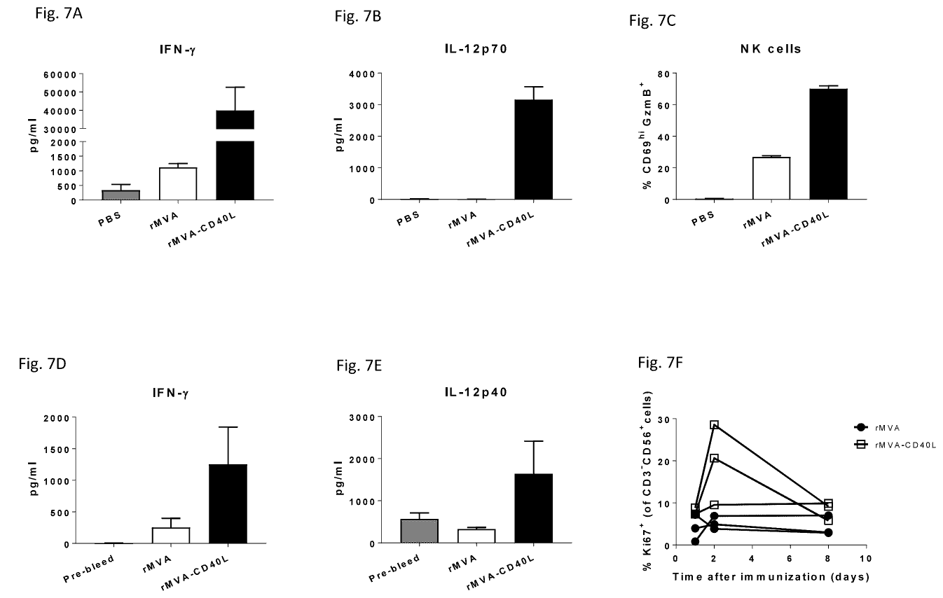

IV administration of recombinant MVA encoding CD40L further enhances NK cell response. In another aspect of the present invention it was determined that an IV administration of the CD40L antigen in addition to the recombinant MVA further enhanced the NK cell response as compared to an IV administration of recombinant MVA alone. As illustrated in FIGS. 1A-7F and 10A-12B, a recombinant MVA encoding a CD40L antigen induced a stronger NK cell response as compared to a recombinant MVA without CD40L in both a single administration and when administered as a homologous prime boost. Further, the quality of the NK cell response was enhanced as compared to the IV administration of the recombinant MVA alone. Increased expression by NK cells of the effector cytokine IFN-γ was observed in all organs analyzed (FIGS. 1A-6F, spleen, liver, lung), as well as expression of CD69 by NK cells in all organs analyzed (FIG. 5C). Moreover, FIGS. 7A-7F shows increased serum levels of IFN-γ 6 hours after IV immunization with rMVA-CD40L compared to recombinant MVA and, more importantly of the NK cell activating cytokine IL-12p70, both in mice and NHPs. In addition, enhanced proliferation of NK cells, demonstrated by the expression of Ki67, was observed not only systemically in mice (FIG. 7B) but also in NHP peripheral blood (FIG. 6F). These results show that IV immunization of rMVA-CD40L compared to rMVA improves NK cell quality in several animal research models.

While recombinant MVA viruses have been previously administered intravenously (see, e.g., WO2002/42480, WO2014/037124), it was previously understood that recombinant MVA administration and treatment was associated with enhancement of an adaptive immune response, such as CD8 T cell responses. For example, in WO2002/42480, CTL responses were measured after immunizations using non-recombinant MVA were done either by IV administration of 10⁷ pfu MVA-BN per mouse, or by subcutaneous administration of 10⁷ pfu or 10⁸ pfu MVA-BN per mouse. In WO2014/037124, mice were intravenously inoculated with recombinant MVA and recombinant MVA encoding CD40L (see, WO2014/037124). CTL responses were enhanced and it was determined that an increased immunogenicity of the recombinant MVA-CD40L was independent of CD4⁺ T cells but dependent upon CD40 in the host.

In at least one aspect, the enhanced NK cell response seen by the present invention is unexpected as it was understood in the art that MVA-induced NK cell activation was shown to be dependent on lymph node-resident CD169-positive subcapsular sinus (SCS) macrophages after subcutaneous immunization (Garcia et al. (2012) Blood 120: 4744-50).

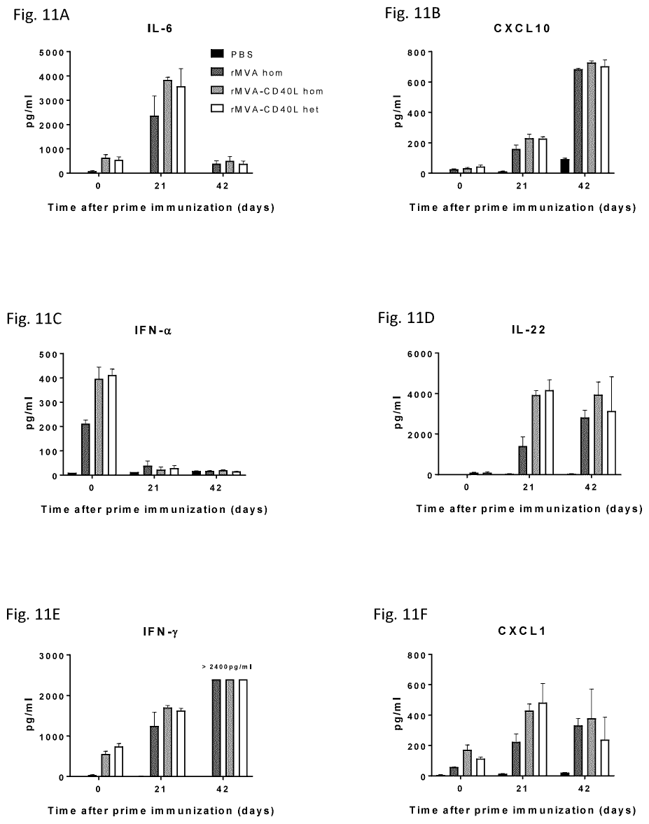

In other aspects, the pharmaceutical combination of the present invention is administered as part of a homologous and/or heterologous prime-boost regimen. Illustrated in FIGS. 10-12, a recombinant MVA encoding CD40L administered to a subject as part of a homologous and/or heterologous prime boost regimen prolongs and reactivates enhanced NK cell responses as well as increases a subject's CD8 and CD4 T cell responses.

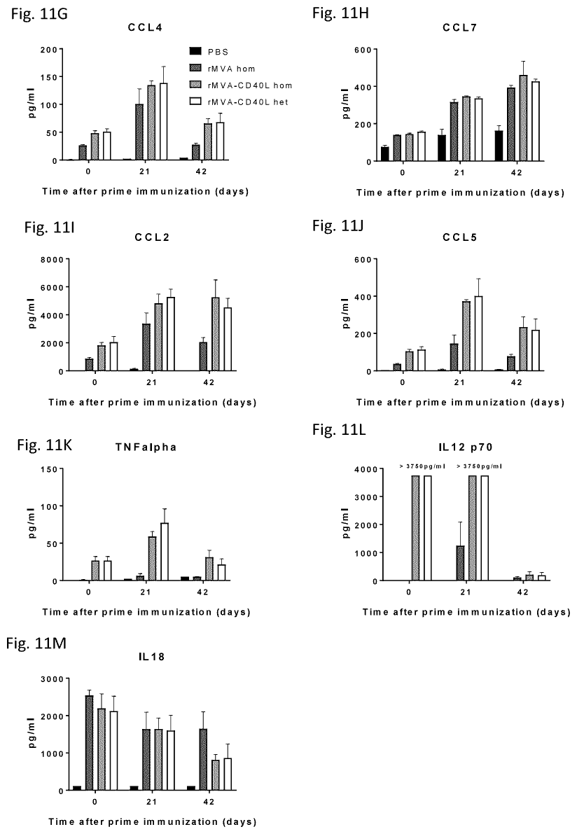

In at least one aspect of the present invention, the enhanced NK cell responses resulting from the repeated recombinant MVA IV administration and the recombinant MVA-CD40L were surprising. In at least one aspect, it was surprising to observe increased NK cell activation and proliferation 24 hours after boost IV immunizations in the absence of an IFN-α increase. Indeed, it was understood that NK cell activation and priming in secondary infections and cancer is largely driven by IFN-α (see, e.g., Stackaruk et al. (2013) Expert Rev. Vaccines. 12(8): 875-84; and Mueller et al. (2017) Front. Immunol. 8: 304). Surprisingly, no increase in IFN-α serum levels were observed 6 hours after rMVA hom, rMVA-CD40L hom or rMVA-CD40L het IV boost immunizations (FIG. 11C). Altogether, repeated homologous or heterologous IV immunizations with rMVA comprising a nucleic acid encoding one or more heterologous antigens resulted in unexpected NK cell activation and proliferation independent of IFN-α.

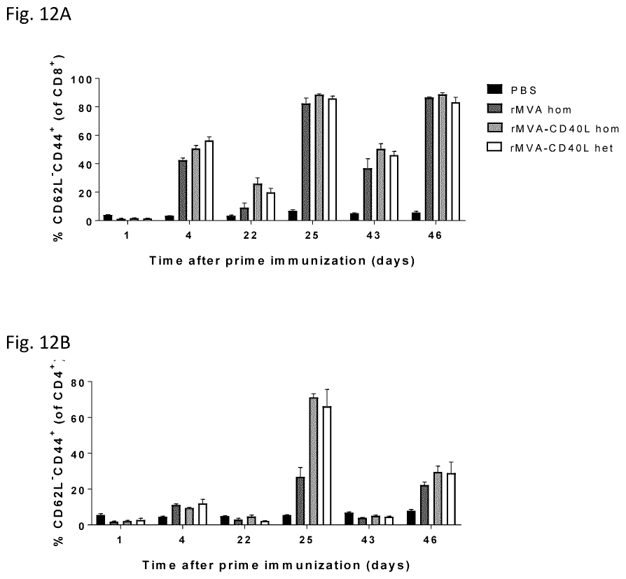

Prior to the present invention, it was understood that CD40L encoded by recombinant MVA can substitute for CD4 T cell help (Lauterbach et al. (2013) Front. Immunol. 4: 251). Further no effect of recombinant MVA-encoded CD40L on CD4 T cells was known. Unexpectedly, we saw expansion of memory CD4⁺ T cells 25 days after prime immunization (FIG. 12B), which corresponds with 4 days after boost IV immunization with rMVA-CD40L (rMVA-CD40L hom and rMVA-CD40L het) (Day 21, see Table 1). This fact is supported by the increased IL-22 production, an important cytokine indicative of T helper cell responses, quantified 6 hours after boost IV immunization in MVA-CD40L hom and MVA-CD40L het groups (FIG. 11D). This unexpected observation is relevant for the maintenance of memory responses by rMVA-CD40L. Furthermore, CD4 T cells can support tumor-specific CD8 T cells at the tumor site, avoid activation-induced cell death and also become cytotoxic themselves (reviewed in Kennedy and Celis (2008) Immunol. Rev. 222: 129-44; Knutson and Disis (2005) Curr. Drug Targets Immune Endocr. Metabol. Disord. 5: 365-71). These results are unexpected because other viral vectors, such as Adenovirus and Herpes Simplex Virus, induce vector-specific immunity that impede the induction of immune responses to the vaccine-encoded antigens upon boost immunization (Lauterbach et al. (2005) J. Gen. Virol. 86: 2401-10; Pine et al. (2011) PLOS One doi: 10.1371/journal.pone.0018526).

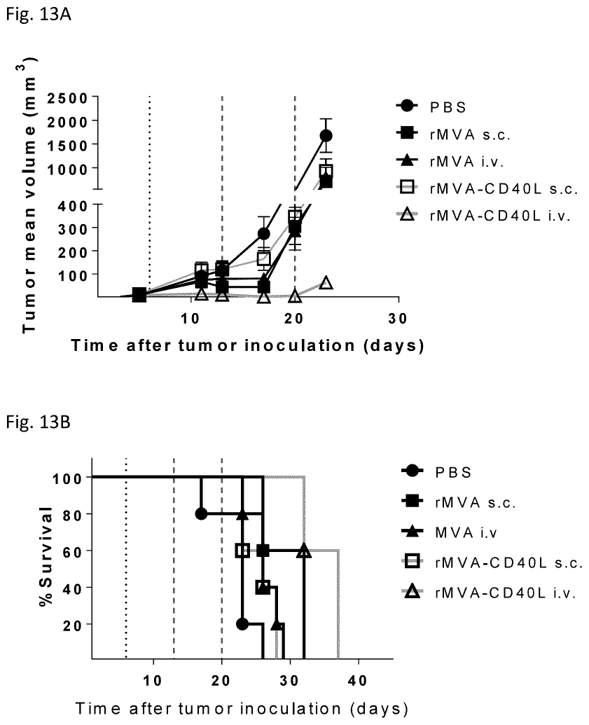

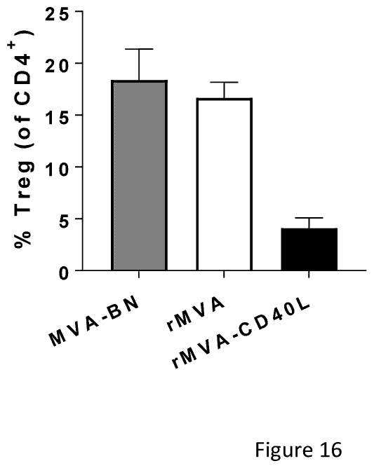

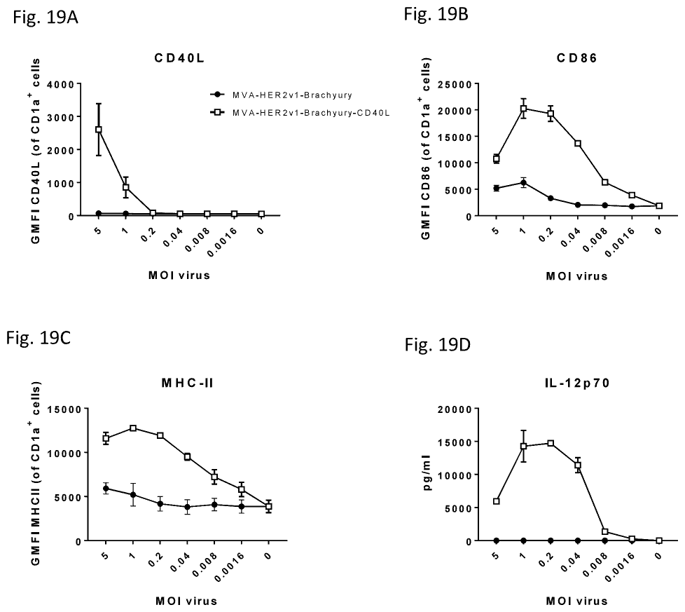

IV administration of MVA reduces a tumor's immunosuppressive effects. Illustrated in FIGS. 13A-15C and 19A-19D, intravenously administered recombinant MVA encoding a heterologous antigen and a CD40L, induced infiltration of CD8⁺ T cells in the tumor and reduced multiple immunosuppressive effects typically employed by tumors to evade the immune system. In addition to increased endogenous CD8⁺ cells within the tumors upon recombinant MVA with or without CD40L challenge, antigen (OVA)-specific T cells were increased in spleen and tumors upon IV administration of a recombinant MVA with CD40L compared to MVA without CD40L. In addition, HER2 antigen-specific T cells producing the effector cytokine IFN-γ were enhanced in the tumor microenvironment upon IV administration of a recombinant MVA with CD40L (FIG. 19A-19D). Surprisingly, immunosuppressive T regulatory cell (Treg) numbers in the tumor microenvironment were decreased when recombinant MVA encoding a heterologous antigen and a CD40L was administered (FIG. 16).

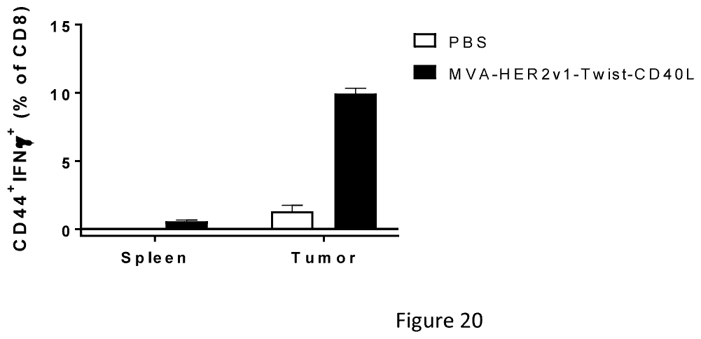

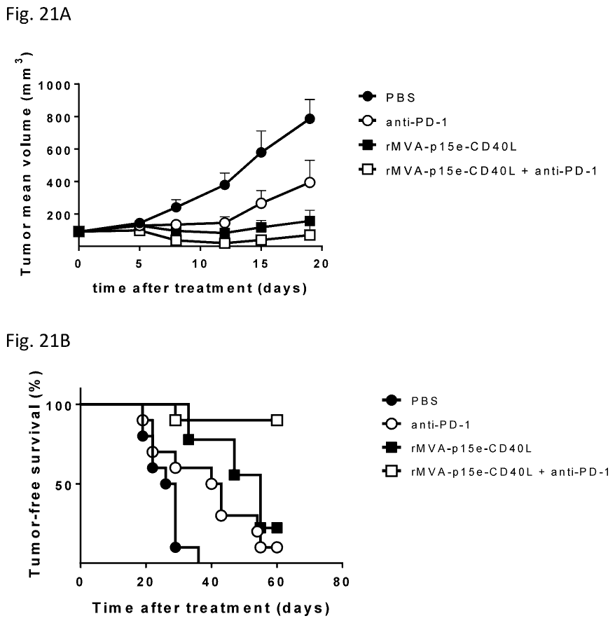

The recombinant MVA encoding CD40L in combination with a checkpoint antagonist or agonist reduces tumor burden and increases survival rate in cancer patients. In various embodiments, the combination includes a) an IV administration of a recombinant MVA encoding a CD40L and b) an administration of an antagonist or agonist of an immune checkpoint molecule. Shown in FIGS. 20 and 21A-21B, the combinations of the present disclosure resulted in a reduction in tumor volume and an increase in overall survival rate. Type I and II interferons, which are induced by both vectors (FIGS. 11C and 11E), are known inducers of PD-1 and PD-L1 expression (reviewed by Dong et al. (2017) Oncotarget 8: 2171-2186). In at least one aspect, the enhanced anti-tumor effects of the pharmaceutical combination (e.g., reduced tumor volume and/or increased survival rate) is achieved from the synergistic combining of tackling the tumor-induced immune suppressive microenvironment via checkpoint blockade and the enhancements of the innate and adaptive T cell responses described herein. In one exemplary embodiment, these enhancements include one or more of those listed above, e.g., an enhanced innate (e.g., NK cell) response, and an enhanced adaptive T cell response.

### Definitions

As used herein, the singular forms “a,” “an,” and “the,” include plural references unless the context clearly indicates otherwise. Thus, for example, reference to “a nucleic acid” includes one or more of the nucleic acids and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. Any of the aforementioned terms (comprising, containing, including, having), though less preferred, whenever used herein in the context of an aspect or embodiment of the present invention can be substituted with the term “consisting of.” When used herein, “consisting of” excludes any element, step, or ingredient not specified in the claim element. When used herein, “consisting essentially of” does not exclude materials or steps that do not materially affect the basic and novel characteristics of the claim.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

“Mutated” or “modified” protein or antigen as described herein is as defined herein any a modification to a nucleic acid or amino acid, such as deletions, additions, insertions, and/or substitutions.

A “host cell” as used herein is a cell that has been introduced with a foreign molecule, virus, or microorganism. In one non-limiting example, as described herein, a cell of a suitable cell culture as, e.g., CEF cells, can be infected with a poxvirus or, in other alternative embodiments, with a plasmid vector comprising a foreign or heterologous gene. Thus, a suitable host cell and cell cultures serve as a host to poxvirus and/or foreign or heterologous gene.

“Percent (%) sequence homology or identity” with respect to nucleic acid sequences described herein is defined as the percentage of nucleotides in a candidate sequence that are identical with the nucleotides in the reference sequence (i.e., the nucleic acid sequence from which it is derived), after aligning the sequences and introducing gaps, if necessary, to achieve the maximum percent sequence identity, and not considering any conservative substitutions as part of the sequence identity. Alignment for purposes of determining percent nucleotide sequence identity or homology can be achieved in various ways that are within the skill in the art, for example, using publicly available computer software such as BLAST, ALIGN, or Megalign (DNASTAR) software. Those skilled in the art can determine appropriate parameters for measuring alignment, including any algorithms needed to achieve maximum alignment over the full length of the sequences being compared.

For example, an appropriate alignment for nucleic acid sequences is provided by the local homology algorithm of Smith and Waterman, (1981), Advances in Applied Mathematics 2:482-489. This algorithm can be applied to amino acid sequences by using the scoring matrix developed by Dayhoff, Atlas of Protein Sequences and Structure, Dayhoff (ed.), 5 suppl. 3: 353-358, National Biomedical Research Foundation, Washington, D.C., USA, and normalized by Gribskov (1986), Nucl. Acids Res. 14(6): 6745-6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: blast.ncbi.nlm.nih.gov/.

The term “prime-boost vaccination” or “prime-boost regimen” refers to a vaccination strategy or regimen using a first priming injection of a vaccine targeting a specific antigen followed at intervals by one or more boosting injections of the same vaccine. Prime-boost vaccination may be homologous or heterologous. A homologous prime-boost vaccination (sometimes referred to herein as “hom”) uses a vaccine comprising the same antigen and vector for both the priming injection and the one or more boosting injections. A heterologous prime-boost vaccination (sometimes referred to herein as “het”) uses a vaccine comprising the same antigen for both the priming injection and the one or more boosting injections but different vectors for the priming injection and the one or more boosting injections. For example, a homologous prime-boost vaccination may use a recombinant poxvirus comprising nucleic acids expressing one or more antigens for the priming injection and the same recombinant poxvirus expressing one or more antigens for the one or more boosting injections. In contrast, a heterologous prime-boost vaccination may use a recombinant poxvirus comprising nucleic acids expressing one or more antigens for the priming injection and a different recombinant poxvirus expressing one or more antigens for the one or more boosting injections.

The term “recombinant” means a polynucleotide, virus or vector of semisynthetic, or synthetic origin which either does not occur in nature or is linked to another polynucleotide in an arrangement not found in nature.

As used herein, reducing or a reduction in tumor volume can be characterized as a reduction in tumor volume and/or size but can also be characterized in terms of clinical trial endpoints understood in the art. Some exemplary clinical trial endpoints associated with a reduction in tumor volume and/or size can include, but are not limited to, Response Rate (RR), Objective response rate (ORR), and so forth.

As used herein an increase in survival rate can be characterized as an increase in survival of a cancer patient, but can also be characterized in terms of clinical trial endpoints understood in the art. Some exemplary clinical trial endpoints associated with an increase in survival rate include, but are not limited to, overall survival rate (OS), Progression free survival (PFS) and so forth.

As used herein, a “transgene” or “heterologous” gene is understood to be a nucleic acid or amino acid sequence which is not present in the wild-type poxviral genome (e.g., Vaccinia, Fowlpox, or MVA). The skilled person understands that a “transgene” or “heterologous gene”, when present in a poxvirus, such as Vaccinia Virus, is to be incorporated into the poxviral genome in such a way that, following administration of the recombinant poxvirus to a host cell, it is expressed as the corresponding heterologous gene product, i.e., as the “heterologous antigen” and\or “heterologous protein.” Expression is normally achieved by operatively linking the heterologous gene to regulatory elements that allow expression in the poxvirus-infected cell. Preferably, the regulatory elements include a natural or synthetic poxviral promoter.

A “vector” refers to a recombinant DNA or RNA plasmid or virus that can comprise a heterologous polynucleotide. The heterologous polynucleotide may comprise a sequence of interest for purposes of prevention or therapy, and may optionally be in the form of an expression cassette. As used herein, a vector needs not be capable of replication in the ultimate target cell or subject. The term includes cloning vectors and viral vectors.

### Combinations and Methods

In various embodiments, the present invention includes a combination for treating a cancer patient by reducing tumor volume and/or increasing survival in the cancer patient. The combination comprises a) a recombinant MVA comprising a first nucleic acid encoding a tumor-associated antigen (TAA) and a second nucleic acid encoding CD40L, that when administered intravenously induces both an enhanced Natural Killer (NK) cell response and an enhanced T cell response as compared to a NK cell response and a T cell response induced by a non-intravenous administration of a recombinant MVA virus comprising a first nucleic acid encoding a TAA and a second nucleic acid encoding CD40L antigen; and b) at least one antagonist or agonist of an immune checkpoint molecule.

### Enhanced NK Cell Response

In one aspect, an “enhanced NK cell response” according to the present disclosure is characterized by one or more of the following: 1) an increase in NK cell frequency, 2) an increase in NK cell activation, and/or 3) an increase in NK cell proliferation. Thus, whether an NK cell response is enhanced in accordance with the present disclosure can be determined by measuring the expression of one or more molecules which are indicative of an increased NK cell frequency, increased NK cell activation, and/or increased NK cell proliferation. Exemplary markers that are useful in measuring NK cell frequency and/or activity include one or more of: NKp46, IFN-γ, CD69, CD70, NKG2D, FasL, granzyme B, CD56, and/or Bcl-X_{L}. Exemplary markers that are useful in measuring NK cell activation include one or more of IFN-γ, CD69, CD70, NKG2D, FasL, granzyme B and/or Bcl-X_{L}. Exemplary markers that are useful in measuring NK cell proliferation include: Ki67. These molecules and the measurement thereof are validated assays that are understood in the art and can be carried out according to known techniques. See, e.g. Borrego et al. ((1999) Immunology 97: 159-165). Additionally, assays for measuring the molecules can be found in Examples 1-3, and 9 of the present disclosure. At least in one aspect, 1) an increase in NK cell frequency can be defined as at least a 2-fold increase in CD3⁻NKp46⁺ cells compared to pre-treatment/baseline; 2) an increase in NK cell activation can be defined as at least a 2-fold increase in IFN-γ, CD69, CD70, NKG2D, FasL, granzyme B and/or Bcl-X_{L} expression compared to pre-treatment/baseline expression; and/or 3) an increase in NK cell proliferation is defined as at least a 1.5 fold increase in Ki67 expression compared to pre-treatment/baseline expression.

### Enhanced T Cell Response

In accordance with the present application, an “enhanced T cell response” is characterized by one or more of the following: 1) an increase in frequency of CD8 T cells; 2) an increase in CD8 T cell activation; and/or 3) an increase in CD8 T cell proliferation. Thus, whether a T cell response is enhanced in accordance with the present application can be determined by measuring the expression of one or more molecules which are indicative of 1) an increase in CD8 T cell frequency 2) an increase in CD8 T cell activation; and/or 3) an increase CD8 T cell proliferation. Exemplary markers that are useful in measuring CD8 T cell frequency, activation, and proliferation include CD3, CD8, IFN-γ, TNF-α, IL-2, CD69 and/or CD44, and Ki67, respectively. Measuring antigen specific T cell frequency can also be measured by ELIspot or MHC Multimers such as pentamers or dextramers as shown by the present application. Such measurements and assays are validated and understood in the art.

In one aspect, an increase in CD8 T cell frequency is characterized by an at least a 2-fold increase in IFN-γ and/or dextramer⁺ CD8 T cells compared to pre-treatment/baseline. An increase in CD8 T cell activation is characterized as at least a 2-fold increase in CD69 and/or CD44 expression compared to pre-treatment baseline expression. An increase in CD8 T cell proliferation is characterized as at least a 2-fold increase in Ki67 expression compared to pre-treatment/baseline expression.

In an alternative aspect, an enhanced T cell response is characterized by an increase in CD8 T cell expression of effector cytokines and/or an increase of cytotoxic effector functions. An increase in expression of effector cytokines can be measured by expression of one or more of IFN-γ, TNF-α, and/or IL-2 compared to pre-treatment/baseline. An increase in cytotoxic effector functions can be measured by expression of one or more of CD107a, granzyme B, and/or perforin and/or antigen-specific killing of target cells.

The assays, cytokines, markers, and molecules described herein and the measurement thereof are validated and understood in the art and can be carried out according to known techniques. Additionally, assays for measuring the T cells responses can be found in Examples 3,7,10 and 15, wherein T cell responses were analyzed.

The enhanced T cell response realized by the present invention is particularly advantageous in combination with the enhanced NK cell response, as the enhanced T cells effectively target and kill those tumor cells that have evaded and/or survived past the initial innate immune responses in the cancer patient. Furthermore, antibody treatment can enhance MHC class I presentation of TAAs, resulting in higher susceptibility of TAA-expressing tumors to lysis by TAA-specific T cells (Kono et al. (2004) Clin. Cancer Res. 10: 2538-44).

In additional embodiments, the combination further comprises at least one antagonist or agonist of an immune checkpoint molecule. In preferred embodiments, the at least one antagonist or agonist of an immune checkpoint molecule comprises a CTLA-4 antagonist, a PD-1 antagonist, a −PD-L1 antagonist, a LAG-3 antagonist, a TIM-3 antagonist, or an ICOS agonist. In more preferred embodiments, the at least one antagonist or agonist of an immune checkpoint molecule comprises a CTLA-4 antagonist, a PD-1 antagonist, or a −PD-L1 antagonist.

In still more embodiments, the at least one of antagonist or agonist of an immune checkpoint molecule comprises an antibody able to block the function of the immune checkpoint molecule. In preferred embodiments, the antibody is selected from CTLA-4 antibody, a PD-1 antibody, a PD-LI antibody, a LAG-3 antibody, an ICOS antibody, and a TIM-3 antibody, respectively. In more preferred embodiments the at least one antagonist or agonist comprises a CTLA-4, a PD-1, or a PD-L1 antibody.

In yet additional embodiments, the combinations and methods described herein are for use in treating a human cancer patient. In preferred embodiments, the cancer patient is suffering from and/or is diagnosed with a cancer selected from the group consisting of: breast cancer, lung cancer, head and neck cancer, thyroid, melanoma, gastric cancer, bladder cancer, kidney cancer, liver cancer, melanoma, pancreatic cancer, prostate cancer, ovarian cancer, urothelial, cervical, or colorectal cancer. In yet additional embodiments, the combinations and methods described herein are for use in treating a human cancer patient suffering from and/or diagnosed with a breast cancer, colorectal cancer or melanoma, preferably a melanoma, more preferably a colorectal cancer or most preferably a colorectal cancer.

### Certain Exemplary Tumor-Associated Antigens

In certain embodiments, an immune response is produced in a subject against a cell-associated polypeptide antigen. In certain such embodiments, a cell-associated polypeptide antigen is a tumor-associated antigen (TAA).

The term “polypeptide” refers to a polymer of two or more amino acids joined to each other by peptide bonds or modified peptide bonds. The amino acids may be naturally occurring as well as non-naturally occurring, or a chemical analogue of a naturally occurring amino acid. The term also refers to proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and or comprise prosthetic groups.

Preferably, the TAA includes, but is not limited to, HER2, PSA, PAP, CEA, MUC-1, survivin, TRP1, TRP2, or Brachyury alone or in combinations. Such exemplary combination may include CEA and MUC-1, also known as CV301. Other exemplary combinations may include PAP and PSA.

Numerous TAAs are known in the art. Exemplary TAAs include, but are not limited to, 5 alpha reductase, alpha-fetoprotein, AM-1, APC, April, BAGE, beta-catenin, Bcl12, bcr-ab1, CA-125, CASP-8/FLICE, Cathepsins, CD19, CD20, CD21, CD23, CD22, CD33 CD35, CD44, CD45, CD46, CD5, CD52, CD55, CD59, CDC27, CDK4, CEA, c-myc, Cox-2, DCC, DcR3, E6/E7, CGFR, EMBP, Dna78, farnesyl transferase, FGF8b, FGF8a, FLK-1/KDR, folic acid receptor, G250, GAGE-family, gastrin 17, gastrin-releasing hormone, GD2/GD3/GM2, GnRH, GnTV, GP1, gp100/Pmel17, gp-100-in4, gp15, gp75/TRP1, hCG, heparanse, Her2/neu, HMTV, Hsp70, hTERT, IGFR1, IL-13R, iNOS, Ki67, KIAA0205, K-ras, H-ras, N-ras, KSA, LKLR-FUT, MAGE-family, mammaglobin, MAP17, melan-A/MART-1, mesothelin, MIC A/B, MT-MMPs, mucin, NY-ESO-1, osteonectin, p15, P170/MDR1, p53, p97/melanotransferrin, PAI-1, PDGF, uPA, PRAME, probasin, progenipoientin, PSA, PSM, RAGE-1, Rb, RCAS1, SART-1, SSX-family, STAT3, STn, TAG-72, TGF-alpha, TGF-beta, Thymosin-beta-15, TNF-alpha, TRP1, TRP2, tyrosinase, VEGF, ZAG, p16INK4, and glutathione-S-transferase.

A preferred PSA antigen comprises the amino acid change of isoleucine to leucine at position 155. See U.S. Pat. No. 7,247,615, which is incorporated herein by reference.

In one or more preferred embodiments of present invention, the heterologous TAA is selected from HER2 and/or Brachyury.

In various additional embodiments, the TAA may include a mutated or modified HER2 antigen selected from HER2v1 and HER2v2. HER2v1 and HER2v2 comprise SEQ ID NO: 1 and SEQ ID NO: 3, respectively. The HER2v1 and HER2v2 antigen may be encoded by nucleic acids comprising SEQ ID NOs: 2 and 4, respectively.

In preferred embodiments, the HER2 antigen comprises an amino acid sequence having at least 90%, 95%, 97% 98%, or 99% identity to SEQ ID NOs:1 or 3. In a most preferred embodiment, the HER2 antigen comprises SEQ ID NOs: 1 or 3.

In additional embodiments, the TAA may include a Brachyury antigen. In preferred embodiments, the Brachyury antigen comprises an amino acid sequence having at least 90%, 95%, 97% 98%, or 99% identity to SEQ ID NOs: 5, 7, 9, or 11. In still additional embodiments, the Brachyury antigen is selected from SEQ ID NOs: 5, 7, 9, and 11, which may be encoded by nucleic acids comprising SEQ ID NOs: 6, 8, 10, and 12, respectively.

### Modified Tumor-Associated Antigens

In certain embodiments, a cell-associated polypeptide antigen is modified such that a CTL response is induced against a cell which presents epitopes derived from a polypeptide antigen on its surface, when presented in association with an MHC Class I molecule on the surface of an APC. In certain such embodiments, at least one first foreign TH epitope, when presented, is associated with an MHC Class II molecule on the surface of the APC. In certain such embodiments, a cell-associated antigen is a tumor-associated antigen.

Exemplary APCs capable of presenting epitopes include dendritic cells and macrophages. Additional exemplary APCs include any pino- or phagocytizing APC, which is capable of simultaneously presenting 1) CTL epitopes bound to MHC class I molecules and 2) TH epitopes bound to MHC class II molecules.

In certain embodiments, modifications to one or more of the TAAs, such as, but not limited to, CEA, MUC-1, PAP, PSA, HER2, survivin, TRP1, TRP2, or Brachyury, are made such that, after administration to a subject, polyclonal antibodies are elicited that predominantly react with the one or more of the TAAs described herein. Such antibodies could attack and eliminate tumor cells as well as prevent metastatic cells from developing into metastases. The effector mechanism of this anti-tumor effect would be mediated via complement and antibody dependent cellular cytotoxicity. In addition, the induced antibodies could also inhibit cancer cell growth through inhibition of growth factor dependent oligo-dimerisation and internalization of the receptors. In certain embodiments, such modified TAAs could induce CTL responses directed against known and/or predicted TAA epitopes displayed by the tumor cells.

In certain embodiments, a modified TAA polypeptide antigen comprises a CTL epitope of the cell-associated polypeptide antigen and a variation, wherein the variation comprises at least one CTL epitope or a foreign TH epitope. Certain such modified TAAs can include in one non-limiting example one or more HER2 polypeptide antigens comprising at least one CTL epitope and a variation comprising at least one CTL epitope of a foreign TH epitope, and methods of producing the same, are described in U.S. Pat. No. 7,005,498 and U.S. Patent Pub. Nos. 2004/0141958 and 2006/0008465.

Certain such modified TAAs can include in one non-limiting example one or more MUC-1 polypeptide antigens comprising at least one CTL epitope and a variation comprising at least one CTL epitope of a foreign epitope, and methods of producing the same, are described in U.S. Patent Pub. Nos. 2014/0363495.

Additional promiscuous T-cell epitopes include peptides capable of binding a large proportion of HLA-DR molecules encoded by the different HLA-DR. See, e.g., WO 98/23635 (Frazer I H et al., assigned to The University of Queensland); Southwood et al. (1998) J. Immunol. 160: 3363 3373; Sinigaglia et al. (1988) Nature 336: 778 780; Rammensee et al. (1995) Immunogenetics 41: 178-228; Chicz et al. (1993) J. Exp. Med. 178: 27-47; Hammer et al. (1993) Cell 74: 197-203; and Falk et al. (1994) Immunogenetics 39: 230-242. The latter reference also deals with HLA-DQ and -DP ligands. All epitopes listed in these references are relevant as candidate natural epitopes as described herein, as are epitopes which share common motifs with these.

In certain other embodiments, the promiscuous T-cell epitope is an artificial T-cell epitope which is capable of binding a large proportion of haplotypes. In certain such embodiments, the artificial T-cell epitope is a pan DR epitope peptide (“PADRE”) as described in WO 95/07707 and in the corresponding paper Alexander et al. (1994) Immunity 1: 751 761.

### CD40L

As illustrated by the present disclosure the inclusion of CD40L as part of the combination and related method further enhances the decrease in tumor volume, prolongs progression-free survival and increase survival rate realized by the present invention. Thus, in various embodiments, the combination further comprises administering CD40L to a cancer patient. In preferred embodiments, the CD40L is encoded as part of a recombinant MVA as described herein.

While CD40 is constitutively expressed on many cell types, including B cells, macrophages, and dendritic cells, its ligand CD40L is predominantly expressed on activated T helper cells. The cognate interaction between dendritic cells and T helper cells early after infection or immunization ‘licenses’ dendritic cells to prime CTL responses. Dendritic cell licensing results in the up-regulation of co-stimulatory molecules, increased survival and better cross-presenting capabilities. This process is mainly mediated via CD40/CD40L interaction. However, various configurations of CD40L are described, from membrane bound to soluble (monomeric to trimeric) which induce diverse stimuli, either inducing or repressing activation, proliferation, and differentiation of APCs.

In one or more preferred embodiments, CD40L is encoded by the MVA of the present invention. In one or more other preferred embodiments, CD40L is a human CD40L. In still more preferred embodiments, the CD40L comprises a nucleic acid having at least 90%, 95%, 97% 98%, or 99% identity to SEQ ID NO:13. In even more preferred embodiments, the CD40L comprises a nucleic acid encoding SEQ ID NO: 13. In a most preferred embodiment, the CD40L comprises SEQ ID NO:13. In additional embodiments, the CD40L is encoded by a nucleic acid having at least 90%, 95%, 97% 98%, or 99% identity to SEQ ID NO:14. In a most preferred embodiment, the nucleic acid comprises SEQ ID NO:14

### Antagonists of Immune Checkpoint Molecules

As described herein, at least in one aspect, the invention encompasses the use of immune checkpoint antagonists. Such immune checkpoint antagonists function to interfere with and/or block the function of the immune checkpoint molecule. Some preferred immune checkpoint antagonists include, Cytotoxic T-Lymphocyte Antigen 4 (CTLA-4), Programmed Cell Death Protein 1 (PD-1), Programmed Death-Ligand 1 (PD-L1), Lymphocyte-activation gene 3 (LAG-3), and T-cell immunoglobulin and mucin domain 3 (TIM-3).

Additionally, exemplary immune checkpoint antagonists can include, but are not limited to CTLA-4, PD-1, PD-L1, PD-L2, LAG-3, TIM-3, T cell Immunoreceptor with Ig and ITIM domains (TIGIT) and V-domain Ig Suppressor of T cell Activation (VISTA).

Such antagonists of the immune checkpoint molecules can include antibodies which specifically bind to immune checkpoint molecules and inhibit and/or block biological activity and function of the immune checkpoint molecule.

Other antagonists of the immune checkpoint molecules can include antisense nucleic acids RNAs that interfere with the expression of the immune checkpoint molecules; and small interfering RNAs that interfere with the expression of the immune checkpoint molecules.

Antagonists can additionally be in the form of small molecules that inhibit or block the function of the immune checkpoint. Some non-limiting examples of these include NP12 (Aurigene), (D) PPA-1 by Tsinghua Univ, high affinity PD-1 (Stanford); BMS-202 and BMS-8 (Bristol Myers Squibb (BMS), and CA170/CA327 (Curis/Aurigene); and small molecule inhibitors of CTLA-4, PD-1, PD-L1, LAG-3, and TIM-3.

Antagonists can additionally be in the form of Anticalins® that inhibit or block the function of the immune checkpoint molecule. See, e.g., Rothe et al. (2018) BioDrugs 32: 233-243.

It is contemplated that antagonists can additionally be in the form of Affimers®. Affimers are Fc Fusion proteins that inhibit or block the function of the immune checkpoint molecule. Other Fusion proteins that can serve as antagonists of immune checkpoints are immune checkpoint fusion proteins (e.g., anti-PD-1 protein AMP-224) and anti-PD-L1 proteins such as those described in US2017/0189476.

Candidate antagonists of immune checkpoint molecules can be screened for function by a variety of techniques known in the art and/or disclosed within the instant application, such as for the ability to interfere with the immune checkpoint molecules function in an in vitro or mouse model.

### Agonist of ICOS

The invention further encompasses agonists of ICOS. An agonist of ICOS activates ICOS. ICOS is a positive co-stimulatory molecule expressed on activated T cells and binding to its ligand promotes their proliferation (Dong (2001) Nature 409: 97-101).

In one embodiment, the agonist is ICOS-L, an ICOS natural ligand. The agonist can be a mutated form of ICOS-L that retains binding and activation properties. Mutated forms of ICOS-L can be screened for activity in stimulating ICOS in vitro.

### Antibodies

In one embodiment, the antagonist and/or agonist of an immune checkpoint molecules each comprises an antibody. Antibodies can be synthetic, monoclonal, or polyclonal and can be made by techniques well known in the art. Such antibodies specifically bind to the immune checkpoint molecule via the antigen-binding sites of the antibody (as opposed to non-specific binding). Immune checkpoint peptides, fragments, variants, fusion proteins, etc., can be employed as immunogens in producing antibodies immunoreactive therewith. More specifically, the polypeptides, fragment, variants, fusion proteins, etc. contain antigenic determinants or epitopes that elicit the formation of antibodies.

In preferred embodiments, the antibodies of present invention are those that are approved, or in the process of approval by the government of a sovereign nation, for the treatment of a human cancer patient. Some non-limiting examples of these antibodies already approved, or in the approval process include the following: CTLA-4(Ipilimumab® and Tremelimumab); PD-1 (Pembrolizumab, Lambrolizumab, Amplimmune-224 (AMP-224), Amplimmune-514 (AMP-514), Nivolumab, MK-3475 (Merck), BI 754091 (Boehringer Ingelheim)), and PD-L1 (Atezolizumab, Avelulmab, Durvalumab, MPDL3280A (Roche), MED14736 (AZN), MSB0010718C (Merck)); LAG-3 (IMP321, BMS-986016, BI754111 (Boehringer Ingelheim), LAG525 (Novartis), MK-4289 (Merck), TSR-033 (Tesaro).

These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (Janeway, Jr. and Travers, ImmunoBiology 3:9 (Garland Publishing Inc., 2nd ed. 1996)). Because folded proteins have complex surfaces, the number of epitopes available is quite numerous; however, due to the conformation of the protein and steric hindrances, the number of antibodies that actually bind to the epitopes is less than the number of available epitopes (Janeway, Jr. and Travers, ImmunoBiology 2 14 (Garland Publishing Inc., 2nd ed. 1996)). Epitopes can be identified by any of the methods known in the art.

Antibodies, including scFV fragments, which bind specifically to the immune checkpoint molecules such as CTLA-4, PD-1, PD-L1, LAG-3, TIM-3, or ICOS and either block its function (“antagonist antibodies”) or enhance/activate its function (“agonist antibodies”), are encompassed by the invention. Such antibodies can be generated by conventional means.

In one embodiment, the invention encompasses monoclonal antibodies against immune checkpoint molecules that either block (“antagonist antibodies”) or enhance/activate (“agonist antibodies”) the function of the immune checkpoint molecules. Exemplary blocking monoclonal antibodies against PD-1 are described in WO 2011/041613, which is hereby incorporated by reference.

Antibodies are capable of binding to their targets with both high avidity and specificity. They are relatively large molecules (˜150 kDa), which can sterically inhibit interactions between two proteins (e.g., PD-1 and its target ligand) when the antibody binding site falls within proximity of the protein-protein interaction site. The invention further encompasses antibodies that bind to epitopes within close proximity to an immune checkpoint molecule ligand binding site.

In various embodiments, the invention encompasses antibodies that interfere with intermolecular interactions (e.g., protein-protein interactions), as well as antibodies that perturb intramolecular interactions (e.g., conformational changes within a molecule). Antibodies can be screened for the ability to block or enhance/activate the biological activity of an immune checkpoint molecule.

Both polyclonal and monoclonal antibodies can be prepared by conventional techniques.

In one exemplary aspect, the immune checkpoint molecules CTLA-4, PD-1, PD-L1, LAG-3, TIM-3, and ICOS and peptides based on the amino acid sequence of CTLA-4, PD-1, PD-L1, LAG-3, TIM-3, and ICOS can be utilized to prepare antibodies that specifically bind to CTLA-4, PD-1, PD-L1, LAG-3, TIM-3, or ICOS. The term “antibodies” is meant to include polyclonal antibodies, monoclonal antibodies, fragments thereof, such as F(ab′)2 and Fab fragments, single-chain variable fragments (scFvs), single-domain antibody fragments (VHHs or Nanobodies), bivalent antibody fragments (diabodies), as well as any recombinantly and synthetically produced binding partners.

In another exemplary aspect, antibodies are defined to be specifically binding if they to an immune checkpoint molecule if they bind with a K_{d} of greater than or equal to about 10⁷ M⁻¹. Affinities of binding partners or antibodies can be readily determined using conventional techniques, for example those described by Scatchard et al. ((1949) Ann. N.Y. Acad. Sci. 51: 660).

Polyclonal antibodies can be readily generated from a variety of sources, for example, horses, cows, goats, sheep, dogs, chickens, rabbits, mice, or rats, using procedures that are well known in the art. In general, purified CTLA-4, PD-1, PD-L1, LAG-3, TIM-3, and ICOS or a peptide based on the amino acid sequence of CTLA-4, PD-1, PD-L1, LAG-3, TIM-3, and ICOS that is appropriately conjugated is administered to the host animal typically through parenteral injection. The immunogenicity of CTLA-4, PD-1, PD-L1,LAG-3, TIM-3, and ICOS can be enhanced through the use of an adjuvant, for example, Freund's complete or incomplete adjuvant. Following booster immunizations, small samples of serum are collected and tested for reactivity to CTLA-4, PD-1, PD-L1, LAG-3, TIM-3, and ICOS polypeptide. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKeam, and Bechtol (eds.), 1980.

For example, the host animals, such as mice, can be injected intraperitoneally at least once and preferably at least twice at about 3 week intervals with isolated and purified immune checkpoint molecule optionally in the presence of adjuvant. Mouse sera are then assayed by conventional dot blot technique or antibody capture (ABC) to determine which animal is best to fuse. Approximately two to three weeks later, the mice are given an intravenous boost of the immune checkpoint molecule. Mice are later sacrificed, and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as a labeled immune checkpoint molecule polypeptide, is added to each well followed by incubation. Positive wells can be subsequently detected. Positive clones can be grown in bulk culture and supernatants are subsequently purified over a Protein A column (Pharmacia).

The monoclonal antibodies of the invention can be produced using alternative techniques, such as those described by Alting-Mees et al. (1990) Strategies in Molecular Biology 3: 1-9, “Monoclonal Antibody Expression Libraries: A Rapid Alternative to Hybridomas,” which is incorporated herein by reference. Similarly, binding partners can be constructed using recombinant DNA techniques to incorporate the variable regions of a gene that encodes a specific binding antibody. Such a technique is described in Larrick et al. ((1989) Biotechnology 7: 394).

Antigen-binding fragments of such antibodies, which can be produced by conventional techniques, are also encompassed by the present invention. Examples of such fragments include, but are not limited to, Fab and F(ab′)2 fragments. Antibody fragments and derivatives produced by genetic engineering techniques are also provided.

The monoclonal antibodies of the present invention include chimeric antibodies, e.g., humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (1988) Nature 332: 323; Liu et al. (1987) Proc. Nat'l. Acad. Sci. 84: 3439; Larrick et al. (1989) Bio/Technology 7: 934; and Winter and Harris (1993) TIPS 14: 139. Procedures to generate antibodies transgenically can be found in GB 2,272,440 and U.S. Pat. Nos. 5,569,825 and 5,545,806, each of which is incorporated by reference herein.

Antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al., International Publication No. WO 87/02671; Akira et al., European Patent Application 0184187; Taniguchi, European Patent Application 0171496; Morrison et al., European Patent Application 0173494; Neuberger et al., PCT International Publication No. WO 86/01533; Cabilly et al., U.S. Pat. No. 4,816,567; Cabilly et al., European Patent Application 0125023; Better et al. (1988) Science 240: 1041-1043; Liu et al. (1987) Proc. Nat'l. Acad. Sci. 84: 3439-3443; Liu et al. (1987) J. Immunol. 139: 3521-3526; Sun et al. (1987) Proc. Nat'l. Acad. Sci. 84: 214-218; Nishimura et al. (1987) Cancer Res. 47: 999-1005; Wood et al. (1985) Nature 314: 446-449; and Shaw et al. (1988) J. Nat'l. Cancer Inst. 80: 1553-1559; Morrison (1985) Science 229: 1202-1207; Oi et al. (1986) BioTechniques 4: 214; Winter, U.S. Pat. No. 5,225,539; Jones et al. (1986) Nature 321: 552-525; Verhoeyan et al. (1988) Science 239: 1534; and Beidler et al. (1988) J. Immunol. 141: 4053-4060.

In connection with synthetic and semi-synthetic antibodies, such terms are intended to cover but are not limited to antibody fragments, isotype switched antibodies, humanized antibodies (e.g., mouse-human, human-mouse), hybrids, antibodies having plural specificities, and fully synthetic antibody-like molecules.

For therapeutic applications, “human” monoclonal antibodies having human constant and variable regions are often preferred so as to minimize the immune response of a patient against the antibody. Such antibodies can be generated by immunizing transgenic animals which contain human immunoglobulin genes. See Jakobovits et al. (1995) Ann. NY Acad. Sci. 764: 525-535.

Human monoclonal antibodies against an immune checkpoint molecule can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al., PCT publication WO 92/01047; Marks et al. (1991) J. Mol. Biol. 222:581-597; and Griffths et al. (1993) EMBO J. 12: 725-734. In addition, a combinatorial library of antibody variable regions can be generated by mutating a known human antibody. For example, a variable region of a human antibody known to bind the immune checkpoint molecule can be mutated, by for example using randomly altered mutagenized oligonucleotides, to generate a library of mutated variable regions which can then be screened to bind to the immune checkpoint molecule. Methods of inducing random mutagenesis within the CDR regions of immunoglobin heavy and/or light chains, methods of crossing randomized heavy and light chains to form pairings and screening methods can be found in, for example, Barbas et al., PCT publication WO 96/07754; Barbas et al. (1992) Proc. Nat'l Acad. Sci. USA 89: 4457-4461.

An immunoglobulin library can be expressed by a population of display packages, preferably derived from filamentous phage, to form an antibody display library. Examples of methods and reagents particularly amenable for use in generating antibody display library can be found in, for example, Ladner et al., U.S. Pat. No. 5,223,409; Kang et al., PCT publication WO 92/18619; Dower et al., PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9: 1370 1372; Hay et al. (1992) Hum. Antibod. Hybridomas 3: 81-85; Huse et al. (1989) Science 246: 1275-1281; Griffiths et al. (1993) supra; Hawkins et al. (1992) J. Mol. Biol. 226: 889-896; Clackson et al. (1991) Nature 352: 624-628; Gram et al. (1992) Proc. Nat'l. Acad. Sci. 89: 3576-3580; Garrad et al. (1991) Bio/Technology 9: 1373-1377; Hoogenboom et al. (1991) Nucl. Acid Res. 19: 4133-4137; and Barbas et al. (1991) Proc. Nat'l. Acad. Sci. 88: 7978-7982. Once displayed on the surface of a display package (e.g., filamentous phage), the antibody library is screened to identify and isolate packages that express an antibody that binds an immune checkpoint molecule.

### Recombinant MVA

In more preferred embodiments of the present invention, the one or more proteins and nucleotides disclosed herein are included in a recombinant MVA. As described and illustrated by the present disclosure, the intravenous administration of the recombinant MVAs of the present disclosure induces in various aspects an enhanced immune response in cancer patients. Thus, in one or more preferred embodiments, the invention includes a recombinant MVA comprising a first nucleic acid encoding one or more of the TAAs described herein and a second nucleic acid encoding CD40L.

Example of MVA virus strains that are useful in the practice of the present invention and that have been deposited in compliance with the requirements of the Budapest Treaty are strains MVA 572, deposited at the European Collection of Animal Cell Cultures (ECACC), Vaccine Research and Production Laboratory, Public Health Laboratory Service, Centre for Applied Microbiology and Research, Porton Down, Salisbury, Wiltshire SP4 0JG, United Kingdom, with the deposition number ECACC 94012707 on Jan. 27, 1994, and MVA 575, deposited under ECACC 00120707 on Dec. 7, 2000, MVA-BN, deposited on Aug. 30, 2000 at the European Collection of Cell Cultures (ECACC) under number V00083008, and its derivatives, are additional exemplary strains.

“Derivatives” of MVA-BN refer to viruses exhibiting essentially the same replication characteristics as MVA-BN, as described herein, but exhibiting differences in one or more parts of their genomes. MVA-BN, as well as derivatives thereof, are replication incompetent, meaning a failure to reproductively replicate in vivo and in vitro. More specifically in vitro, MVA-BN or derivatives thereof have been described as being capable of reproductive replication in chicken embryo fibroblasts (CEF), but not capable of reproductive replication in the human keratinocyte cell line HaCat (Boukamp et al. (1988) J. Cell Biol. 106: 761-771), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, MVA-BN or derivatives thereof have a virus amplification ratio at least two-fold less, more preferably three-fold less than MVA-575 in Hela cells and HaCaT cell lines. Tests and assay for these properties of MVA-BN and derivatives thereof are described in WO 02/42480 (U.S. Patent Application No. 2003/0206926) and WO 03/048184 (U.S. Patent Application No. 2006/0159699).

The term “not capable of reproductive replication” or “no capability of reproductive replication” in human cell lines in vitro as described in the previous paragraphs is, for example, described in WO 02/42480, which also teaches how to obtain MVA having the desired properties as mentioned above. The term applies to a virus that has a virus amplification ratio in vitro at 4 days after infection of less than 1 using the assays described in WO 02/42480 or in U.S. Pat. No. 6,761,893.

The term “failure to reproductively replicate” refers to a virus that has a virus amplification ratio in human cell lines in vitro as described in the previous paragraphs at 4 days after infection of less than 1. Assays described in WO 02/42480 or in U.S. Pat. No. 6,761,893 are applicable for the determination of the virus amplification ratio.

The amplification or replication of a virus in human cell lines in vitro as described in the previous paragraphs is normally expressed as the ratio of virus produced from an infected cell (output) to the amount originally used to infect the cell in the first place (input) referred to as the “amplification ratio”. An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1, i.e., a decrease in output compared to the input level, indicates a lack of reproductive replication and therefore attenuation of the virus.

### Expression Cassettes/Control Sequences

In various aspects, the one or more nucleic acids described herein are embodied in in one or more expression cassettes in which the one or more nucleic acids are operatively linked to expression control sequences. “Operably linked” means that the components described are in relationship permitting them to function in their intended manner, e.g., a promoter to transcribe the nucleic acid to be expressed. An expression control sequence operatively linked to a coding sequence is joined such that expression of the coding sequence is achieved under conditions compatible with the expression control sequences. The expression control sequences include, but are not limited to, appropriate promoters, enhancers, transcription terminators, a start codon at the beginning a protein-encoding open reading frame, splicing signals for introns, and in-frame stop codons. Suitable promoters include, but are not limited to, the SV40 early promoter, an RSV promoter, the retrovirus LTR, the adenovirus major late promoter, the human CMV immediate early I promoter, and various poxvirus promoters including, but not limited to the following vaccinia virus or MVA-derived and FPV-derived promoters: the 30K promoter, the I3 promoter, the PrS promoter, the PrS5E promoter, the Pr7.5K, the PrHyb promoter, the Pr13.5 long promoter, the 40K promoter, the MVA-40K promoter, the FPV 40K promoter, 30k promoter, the PrSynIIm promoter, the PrLE1 promoter, and the PR1238 promoter. Additional promoters are further described in WO 2010/060632, WO 2010/102822, WO 2013/189611, WO 2014/063832, and WO 2017/021776 which are incorporated fully by reference herein.

Additional expression control sequences include, but are not limited to, leader sequences, termination codons, polyadenylation signals and any other sequences necessary for the appropriate transcription and subsequent translation of the nucleic acid sequence encoding the desired recombinant protein (e.g., HER2, Brachyury, and/or CD40L) in the desired host system. The poxvirus vector may also contain additional elements necessary for the transfer and subsequent replication of the expression vector containing the nucleic acid sequence in the desired host system. It will further be understood by one skilled in the art that such vectors are easily constructed using conventional methods (Ausubel et al. (1987) in “Current Protocols in Molecular Biology,” John Wiley and Sons, New York, N.Y.) and are commercially available.

### Methods and Dosing regimens for administering the Combination

In one or more aspects, the combinations of the present invention can be administered as part of a homologous and/or heterologous prime-boost regimen. Illustrated in FIGS. 10A-12B, a homologous and/or heterologous prime boost regimen prolongs and reactivates enhanced NK cell responses as well as increases a subject's specific CD8 and CD4 T cell responses. Thus, in one or more embodiments there is a combination and/or method for a reducing tumor size and/or increasing survival in a cancer patient comprising administering to the cancer patient a combination of the present disclosure, wherein the combination is administered as part of a homologous or heterologous prime-boost regimen.

### Generation of Recombinant MVA Viruses Comprising Transgenes

The recombinant MVA viruses provided herein can be generated by routine methods known in the art. Methods to obtain recombinant poxviruses or to insert exogenous coding sequences into a poxviral genome are well known to the person skilled in the art. For example, methods for standard molecular biology techniques such as cloning of DNA, DNA and RNA isolation, Western blot analysis, RT-PCR and PCR amplification techniques are described in “Molecular Cloning, A Laboratory Manual” (2nd Ed.) (J. Sambrook et al., Cold Spring Harbor Laboratory Press (1989), and techniques for the handling and manipulation of viruses are described in “Virology Methods Manual” (Mahy et al. (eds.), Academic Press (1996)). Similarly, techniques and know-how for the handling, manipulation and genetic engineering of MVA are described in “Molecular Virology: A Practical Approach” (Davison & Elliott (eds.), The Practical Approach Series, IRL Press at Oxford University Press, Oxford, UK (1993) (see, e.g., Chapter 9: Expression of genes by Vaccinia virus vectors) and “Current Protocols in Molecular Biology” (John Wiley & Son, Inc. (1998), see, e.g., Chapter 16, Section IV: “Expression of proteins in mammalian cells using vaccinia viral vector”).

For the generation of the various recombinant MVA viruses disclosed herein, different methods may be applicable. The DNA sequence to be inserted into the virus can be placed into an E. coli plasmid construct into which DNA homologous to a section of DNA of the poxvirus has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of poxviral DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA virus. Recombination between homologous MVA viral DNA in the plasmid and the viral genome, respectively, can generate a poxvirus modified by the presence of foreign DNA sequences.

According to a preferred embodiment, a cell of a suitable cell culture as, e.g., CEF cells, can be infected with a MVA virus. The infected cell can be, subsequently, transfected with a first plasmid vector comprising a foreign or heterologous gene or genes, such as one or more of the nucleic acids provided in the present disclosure; preferably under the transcriptional control of a poxvirus expression control element. As explained above, the plasmid vector also comprises sequences capable of directing the insertion of the exogenous sequence into a selected part of the MVA viral genome. Optionally, the plasmid vector also contains a cassette comprising a marker and/or selection gene operably linked to a poxviral promoter. Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In case, this gene shall be introduced into a different insertion site of the poxviral genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can at first be transfected by the plasmid vector comprising the foreign gene and, then, infected with the poxvirus. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E.coli or another bacterial species between a MVA virus genome cloned as a bacterial artificial chromosome (BAC) and a linear foreign sequence flanked with DNA sequences homologous to sequences flanking the desired site of integration in the MVA virus genome.

The one or more nucleic acids of the present disclosure may be inserted into any suitable part of the MVA virus or MVA viral vector. Suitable parts of the MVA virus are non-essential parts of the MVA genome. Non-essential parts of the MVA genome may be intergenic regions or the known deletion sites 1-6 of the MVA genome. Alternatively, or additionally, non-essential parts of the recombinant MVA can be a coding region of the MVA genome which is non-essential for viral growth. However, the insertion sites are not restricted to these preferred insertion sites in the MVA genome, since it is within the scope of the present invention that the nucleic acids of the present invention (e.g., HER2, Brachyury, and CD40L) and any accompanying promoters as described herein may be inserted anywhere in the viral genome as long as it is possible to obtain recombinants that can be amplified and propagated in at least one cell culture system, such as Chicken Embryo Fibroblasts (CEF cells).

Preferably, the nucleic acids of the present invention may be inserted into one or more intergenic regions (IGR) of the MVA virus. The term “intergenic region” refers preferably to those parts of the viral genome located between two adjacent open reading frames (ORF) of the MVA virus genome, preferably between two essential ORFs of the MVA virus genome. For MVA, in certain embodiments, the IGR is selected from IGR 07/08, IGR 44/45, IGR 64/65, IGR 88/89, IGR 136/137, and IGR 148/149.

For MVA virus, the nucleotide sequences may, additionally or alternatively, be inserted into one or more of the known deletion sites, i.e., deletion sites I, II, III, IV, V, or VI of the MVA genome. The term “known deletion site” refers to those parts of the MVA genome that were deleted through continuous passaging on CEF cells characterized at passage 516 with respect to the genome of the parental virus from which the MVA is derived from, in particular the parental chorioallantois vaccinia virus Ankara (CVA) e.g., as described in Meisinger-Henschel et al. (2007) J. Gen. Virol. 88: 3249-3259.

### Vaccines

In certain embodiments, the recombinant MVA of the present disclosure can be formulated as part of a vaccine. For the preparation of vaccines, the MVA virus can be converted into a physiologically acceptable form.

An exemplary preparation follows. Purified virus is stored at −80° C. with a titer of 5×10⁸ TCID50/ml formulated in 10 mM Tris, 140 mM NaCl, pH 7.4. For the preparation of vaccine shots, e.g., 1×10⁸-1×10⁹ particles of the virus can be lyophilized in phosphate-buffered saline (PBS) in the presence of 2% peptone and 1% human albumin in an ampoule, preferably a glass ampoule. Alternatively, the vaccine shots can be prepared by stepwise, freeze-drying of the virus in a formulation. In certain embodiments, the formulation contains additional additives such as mannitol, dextran, sugar, glycine, lactose, polyvinylpyrrolidone, or other additives, such as, including, but not limited to, antioxidants or inert gas, stabilizers or recombinant proteins (e.g., human serum albumin) suitable for in vivo administration. The ampoule is then sealed and can be stored at a suitable temperature, for example, between 4° C. and room temperature for several months. However, as long as no need exists, the ampoule is stored preferably at temperatures below −20° ° C., most preferably at about −80° C.

In various embodiments involving vaccination or therapy, the lyophilisate is dissolved in 0.1 to 0.5 ml of an aqueous solution, preferably physiological saline or Tris buffer such as 10 mM Tris, 140 mM NaCl pH 7.7. It is contemplated that the recombinant MVA, vaccine or pharmaceutical composition of the present disclosure can be formulated in solution in a concentration range of 10⁴ to 10¹⁰ TCID₅₀/ml, 10⁵ to 5×10⁹ TCID₅₀/ml, 10⁶ to 5×10⁹ TCID₅₀/ml, or 10⁷ to 5×10⁹ TCID₅₀/ml. A preferred dose for humans comprises between 10⁶ to 10¹⁰ TCID₅₀, including a dose of 10⁶ TCID₅₀, 10⁷ TCID₅₀, 10⁸ TCID₅₀, 5×10⁸TCID₅₀, 10⁹ TCID₅₀, 5×10⁹ TCID₅₀, or 10¹⁰ TCID₅₀. Optimization of dose and number of administrations is within the skill and knowledge of one skilled in the art.

In one or more preferred embodiments, as set forth herein, the recombinant MVA is administered to a cancer patient intravenously.

In additional embodiments, the immune checkpoint antagonist or agonist, or preferably antibody can be administered either systemically or locally, i.e., by intraperitoneal, parenteral, subcutaneous, intravenous, intramuscular, intranasal, intradermal, or any other path of administration known to a skilled practitioner.

### Kits, Compositions, and Methods of Use

In various embodiments, the invention encompasses kits, pharmaceutical combinations, pharmaceutical compositions, and/or immunogenic combination, comprising the a) recombinant MVA that includes the nucleic acids described herein and b) one or more antibodies described herein.

It is contemplated that the kit and/or composition can comprise one or multiple containers or vials of a recombinant poxvirus of the present disclosure, one or more containers or vials of an antibody of the present disclosure, together with instructions for the administration of the recombinant MVA and antibody. It is contemplated that in a more particular embodiment, the kit can include instructions for administering the recombinant MVA and antibody in a first priming administration and then administering one or more subsequent boosting administrations of the recombinant MVA and antibody.

The kits and/or compositions provided herein may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

### CERTAIN EXEMPLARY EMBODIMENTS

Embodiment 1 is a combination, or pharmaceutical combination, for use in reducing tumor size and/or increasing survival in a cancer patient, the combination comprising: a) a recombinant modified Vaccinia Ankara (MVA) virus comprising a first nucleic acid encoding a heterologous tumor-associated antigen (TAA) and a second nucleic acid encoding CD40 Ligand (CD40L), that when administered intravenously induces both an enhanced Natural Killer (NK) cell response and an enhanced T cell response as compared to an NK cell response and a T cell response induced by a non-intravenous administration of a recombinant MVA virus comprising a first nucleic acid encoding a TAA and a second nucleic acid encoding CD40L; and b) at least one antagonist or agonist of an immune checkpoint molecule wherein (a) and (b) are to be administered as a combination treatment; and wherein administration of a) and b) to the cancer patient reduces tumor size and/or increases the survival rate of the cancer patient as compared to a non-IV administration of a) or an administration of b) alone.

Embodiment 2 is a method for reducing tumor size and/or increasing survival in a cancer patient comprising: a) administering to the cancer patient a) administering a recombinant modified Vaccinia Ankara (MVA) virus comprising a first nucleic acid encoding a heterologous TAA and a second nucleic acid encoding CD40L, that when administered intravenously induces both an enhanced Natural Killer (NK) cell response and an enhanced T cell response as compared to an NK cell response and a T cell response induced by a non-intravenous administration of a recombinant MVA virus comprising a nucleic acid encoding a CD40L; and administering to the cancer patient b) at least one of an antagonist or agonist of an immune checkpoint molecule; wherein (a) and (b) are to be administered as a combination treatment; and wherein administration of a) and b) to the cancer patient reduces tumor size and/or increases the survival rate of the cancer patient as compared to a non-IV administration of a) or an administration of b) alone.

Embodiment 3 is a combination therapy for reducing tumor size and/or increasing survival in a cancer patient, the combination comprising: a) a recombinant modified Vaccinia Ankara (MVA) virus comprising a first nucleic acid encoding a heterologous TAA and a second nucleic acid encoding CD40L, that when administered intravenously induces both an enhanced Natural Killer (NK) cell response and an enhanced T cell response as compared to an NK cell response and a T cell response induced by a non-intravenous administration of a recombinant MVA virus comprising a nucleic acid encoding a CD40L; and b) at least one of an antagonist or agonist of an immune checkpoint molecule; wherein (a) and (b) are to be administered as a combination treatment; and wherein administration of a) and b) to the cancer patient reduces tumor size and/or increases the survival rate of the cancer patient as compared to a non-IV administration of a) or an administration of b) alone.

Embodiment 4, is a combination for use, a method, and/or combination therapy of any one of Embodiments 1-3, wherein the antagonist or agonist of an immune checkpoint molecule comprises an antibody to the immune checkpoint molecule.

Embodiment 5, is a combination for use, a method, and/or combination therapy of any one of Embodiments 1-4, wherein the antagonist or agonist of an immune checkpoint molecule comprises an a CTLA-4 antagonist, a PD-1 antagonist, a PD-L1 antagonist, a LAG-3 antagonist, a TIM-3 antagonist, or an ICOS agonist.

Embodiment 6, is a combination for use, a method, and/or combination therapy of any one of Embodiments 1-5, wherein the antagonist or agonist of an immune checkpoint molecule comprises an a CTLA-4 antibody, a PD-1 antibody, a PD-L1 antibody, a LAG-3 antibody, a TIM-3 antibody, or an ICOS antibody.

Embodiment 7, is a combination for use, a method, and/or combination therapy of any one of Embodiments 1-6, wherein the antagonist or agonist of an immune checkpoint molecule comprises an a CTLA-4 antibody, a PD-1 antibody, and/or a PD-L1 antibody.

Embodiment 8, is a combination for use, a method, and/or combination therapy of any one of Embodiments 1-7, wherein the antagonist or agonist of an immune checkpoint molecule comprises a PD-1 antibody and/or a PD-L1 antibody.

Embodiment 9 is a combination for use, a method, and/or combination therapy of Embodiments 1-8, wherein b) is a PD-1 antibody.

Embodiment 10 is a combination for use, a method, and/or combination therapy of any one of Embodiments 1-9, wherein the recombinant MVA further comprises a second nucleic acid encoding a heterologous tumor-associated antigen (TAA).

Embodiment 11 is a combination for use, a method, and/or combination therapy of Embodiment 1-10, wherein the heterologous tumor-associated antigen (TAA) is selected from the group consisting of: carcinoembryonic antigen (CEA), Mucin 1, cell surface associated (MUC-1), Prostatic Acid Phosphatase (PAP), Prostate Specific Antigen (PSA), human epidermal growth factor receptor 2 (HER2), survivin, tyrosine related protein 1 (TRP1), tyrosine related protein 2 (TRP2), Brachyury antigen, or combinations thereof.

Embodiment 12 is a combination for use, a method, and/or combination therapy of Embodiment 1-11, wherein the heterologous tumor-associated antigen (TAA) is selected from the group consisting of: carcinoembryonic antigen (CEA), Mucin 1, cell surface associated (MUC-1).

Embodiment 13 is a combination for use, a method, and or combination therapy of any one of Embodiments 1-12, wherein the heterologous tumor-associated antigen (TAA) is human epidermal growth factor receptor 2 (HER2).

Embodiment 14 is a combination for use, a method, and/or combination therapy of Embodiment 1-13, wherein the TAA is selected from the group consisting of: 5-α-reductase, α-fetoprotein (AFP), AM-1, APC, April, B melanoma antigen gene (BAGE), β-catenin, Bcl12, bcr-ab1, Brachyury, CA-125, caspase-8 (CASP-8), Cathepsins, CD19, CD20, CD21/complement receptor 2 (CR2), CD22/BL-CAM, CD23/FcεERII, CD33, CD35/complement receptor 1 (CR1), CD44/PGP-1, CD45/leucocyte common antigen (LCA), CD46/membrane cofactor protein (MCP), CD52/CAMPATH-1, CD55/decay accelerating factor (DAF), CD59/protectin, CDC27, CDK4, carcinoembryonic antigen (CEA), c-myc, cyclooxygenase-2 (cox-2), deleted in colorectal cancer gene (DCC), DcR3, E6/E7, CGFR, EMBP, Dna78, farnesyl transferase, fibroblast growth factor-8a (FGF8a), fibroblast growth factor-8b (FGF8b), FLK-1/KDR, folic acid receptor, G250, G melanoma antigen gene family (GAGE-family), gastrin 17, gastrin-releasing hormone, ganglioside 2 (GD2)/ganglioside 3 (GD3)/ganglioside-monosialic acid-2 (GM2), gonadotropin releasing hormone (GnRH), UDP-GlcNAc:R1Man(α1-6)R2 [GlcNAc to Man(α1-6)] β1,6-N¬-acetylglucosaminyltransferase V (GnT V), GP1, gp100/Pme117, gp-100-in4, gp15, gp75/tyrosine-related protein-1 (gp75/TRP1), human chorionic gonadotropin (hCG), heparanase, HER2, human mammary tumor virus (HMTV), 70 kiloDalton heat-shock protein (“HSP70”), human telomerase reverse transcriptase (hTERT), insulin-like growth factor receptor-1 (IGFR-1), interleukin-13 receptor (IL-13R), inducible nitric oxide synthase (“iNOS”), Ki67, KIAA0205, K-ras, H-ras, N-ras, KSA, LKLR-FUT, melanoma antigen-encoding gene 1 (MAGE-1), melanoma antigen-encoding gene 2 (MAGE-2), melanoma antigen-encoding gene 3 (MAGE-3), melanoma antigen-encoding gene 4 (MAGE-4), mammaglobin, MAP17, Melan-A/melanoma antigen recognized by T-cells-1 (MART-1), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (PDGF), μPA, PRAME, probasin, progenipoietin, prostate-specific antigen (PSA), prostate-specific membrane antigen (PSMA), RAGE-1, Rb, RCAS1, SART-1, SSX-family, STAT3, STn, TAG-72, transforming growth factor-alpha (TGF-α), transforming growth factor-beta (TGF-β), Thymosin-beta-15, tumor necrosis factor-alpha (“TNF-α”), TRP1, TRP2, tyrosinase, vascular endothelial growth factor (VEGF), ZAG, p16INK4, and glutathione-S-transferase (GST)

Embodiment 15 is a combination for use, a method, and/or combination therapy of any one of Embodiments 1-14, wherein the MVA is MVA-BN or a derivative of MVA-BN.

Embodiment 16 is a combination for use, a method, and/or combination therapy of any one of Embodiments 1-15, wherein a) is administered at the same time as or prior to b).

Embodiment 17 is a combination for use, a method, and/or combination therapy of any one of Embodiments 1-16, wherein a) and b) are administered to the cancer patient in a priming administration followed by one or more boosting administrations of a) and b) to the cancer patient.

Embodiment 18 is a combination for use, a method, and/or combination therapy of any one of Embodiments 1-17, wherein the cancer patient is suffering from and/or is diagnosed with a cancer selected from the group consisting of: breast cancer, lung cancer, head and neck cancer, thyroid, melanoma, gastric cancer, bladder cancer, kidney cancer, liver cancer, melanoma, pancreatic cancer, prostate cancer, ovarian cancer, or colorectal cancer.

Embodiment 19 is a combination for use, a method, and/or combination therapy of Embodiment 18, wherein the breast cancer is a HER2 overexpressing breast cancer.

Embodiment 20 is a combination for use, a method, and/or combination therapy of Embodiment 19, wherein the HER2 antigen has at least 90%, 95%, 97% 98%, or 99% identity to SEQ ID NO:1 or SEQ ID NO:3.

Embodiment 21 is a combination for use, a method, and/or combination therapy of Embodiment 19, wherein the HER2 antigen has at least 90%, 95%, 97% 98%, or 99% identity to SEQ ID NO:1 or SEQ ID NO:3.

Embodiment 22 is a combination for use, a method, and/or combination therapy of Embodiment 19, wherein the HER2 antigen comprises SEQ ID NO:1 or SEQ ID NO:3.

Embodiment 23 is a combination for use, a method, and/or combination therapy of Embodiment 11-13, wherein the Brachyury antigen comprises an amino acid sequence having at least 90%, 95%, 97% 98%, or 99% identity to SEQ ID NO: 5, SEQ ID NO: 7, SEQ ID NO: 9, or SEQ ID NO: 11.

Embodiment 24 is use of the combination of any one of Embodiments 1-23 in the preparation of a pharmaceutical or medicament for reducing tumor volume and/or increasing survival of a cancer patient.

Embodiment 25 is a pharmaceutical combination comprising:
a) a recombinant modified Vaccinia Ankara (MVA) virus comprising a first nucleic acid encoding a heterologous tumor associated antigen (TAA) and a second nucleic acid encoding CD40L; and b) at least one antagonist or agonist of an immune checkpoint molecule.

Embodiment 26 is a combination according to Embodiment 25, wherein the antagonist or agonist of an immune checkpoint molecule comprises a CTLA-4 antagonist, a PD-1 antagonist, a PD-L1 antagonist, a LAG-3 antagonist, a TIM-3 antagonist, or an ICOS agonist.

Embodiment 27 is a combination according to Embodiments 25-26, wherein the antagonist or agonist of an immune checkpoint molecule comprises a CTLA-4 antagonist, a PD-1 antagonist, or a PD-L1 antagonist.

Embodiment 28 is a combination according to Embodiments 25-27, wherein the antagonist or agonist of an immune checkpoint molecule comprises a CTLA-4 antagonist, a PD-1 antagonist, or a PD-L1 antagonist.

Embodiment 29 is a combination according to Embodiments 25-28, wherein the antagonist or agonist of an immune checkpoint molecule comprises a PD-1 antagonist, or a PD-L1 antagonist.

Embodiment 30 is a combination according to Embodiments 25-29, wherein the antagonist or agonist of an immune checkpoint molecule comprises an antibody.

Embodiment 31 is a combination according to Embodiments 25-30, wherein the CTLA-4 antagonist, the PD-1 antagonist, the PD-L1 antagonist, the LAG-3 antagonist, the TIM-3 antagonist, and the ICOS agonist comprisea CTLA-4 antibody, a PD-1 antibody, a PD-L1 antibody, a LAG-3 antibody, and an ICOS antibody, respectively.

Embodiment 32 is a combination according to Embodiments 25-31, wherein the antagonist or agonist of an immune checkpoint molecule comprisesa PD-1 antibody or PD-L1 antibody.

Embodiment 33 is a combination according to Embodiments 25-32, wherein the heterologous tumor-associated antigen (TAA) is selected from the group consisting of: carcinoembryonic antigen (CEA), Mucin 1, cell surface associated (MUC-1), Prostatic Acid Phosphatase (PAP), Prostate Specific Antigen (PSA), human epidermal growth factor receptor 2 (HER2), survivin, tyrosine related protein 1 (TRP1), tyrosine related protein 2 (TRP2), Brachyury antigen, or combinations thereof.

Embodiment 34 is a combination according to Embodiments 25-33, wherein the heterologous tumor-associated antigen (TAA) is selected from the group consisting of: carcinoembryonic antigen (CEA), Mucin 1, cell surface associated (MUC-1).

Embodiment 35 is a combination according to Embodiments 25-34, wherein the heterologous tumor-associated antigen (TAA) is human epidermal growth factor receptor 2 (HER2).

Embodiment 36 is a combination according to Embodiments 25-34, wherein the TAA is selected from the group consisting of: 5-α-reductase, α-fetoprotein (AFP), AM-1, APC, April, B melanoma antigen gene (BAGE), β-catenin, Bcl12, ber-ab1, Brachyury, CA-125, caspase-8 (CASP-8), Cathepsins, CD19, CD20, CD21/complement receptor 2 (CR2), CD22/BL-CAM, CD23/F_{c}εRII, CD33, CD35/complement receptor 1 (CR1), CD44/PGP-1, CD45/leucocyte common antigen (LCA), CD46/membrane cofactor protein (MCP), CD52/CAMPATH-1, CD55/decay accelerating factor (DAF), CD59/protectin, CDC27, CDK4, carcinoembryonic antigen (CEA), c-myc, cyclooxygenase-2 (cox-2), deleted in colorectal cancer gene (DCC), DcR3, E6/E7, CGFR, EMBP, Dna78, farnesyl transferase, fibroblast growth factor-8a (FGF8a), fibroblast growth factor-8b (FGF8b), FLK-1/KDR, folic acid receptor, G250, G melanoma antigen gene family (GAGE-family), gastrin 17, gastrin-releasing hormone, ganglioside 2 (GD2)/ganglioside 3 (GD3)/ganglioside-monosialic acid-2 (GM2), gonadotropin releasing hormone (GnRH), UDP-GlcNAc:R₁Man(α1-6)R₂[GlcNAc to Man(α1-6)] β1,6-N-acetylglucosaminyltransferase V (GnT V), GP1, gp100/Pme117, gp-100-in4, gp15, gp75/tyrosine-related protein-1 (gp75/TRP1), human chorionic gonadotropin (hCG), heparanase, HER2, human mammary tumor virus (HMTV), 70 kiloDalton heat-shock protein (“HSP70”), human telomerase reverse transcriptase (hTERT), insulin-like growth factor receptor-1 (IGFR-1), interleukin-13 receptor (IL-13R), inducible nitric oxide synthase (“iNOS”), Ki67, KIAA0205, K-ras, H-ras, N-ras, KSA, LKLR-FUT, melanoma antigen-encoding gene 1 (MAGE-1), melanoma antigen-encoding gene 2 (MAGE-2), melanoma antigen-encoding gene 3 (MAGE-3), melanoma antigen-encoding gene 4 (MAGE-4), mammaglobin, MAP17, Melan-A/melanoma antigen recognized by T-cells-1 (MART-1), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (PDGF), μPA, PRAME, probasin, progenipoietin, prostate-specific antigen (PSA), prostate-specific membrane antigen (PSMA), RAGE-1, Rb, RCAS1, SART-1, SSX-family, STAT3, STn, TAG-72, transforming growth factor-alpha (TGF-α), transforming growth factor-beta (TGF-β), Thymosin-beta-15, tumor necrosis factor-alpha (“TNF-α”), TRP1, TRP2, tyrosinase, vascular endothelial growth factor (VEGF), ZAG, p16INK4, and glutathione-S-transferase (GST)

Embodiment 37 is a combination according to Embodiments 25-36, wherein the MVA is MVA-BN or a derivative of MVA-BN.

Embodiment 38 is a combination according to Embodiments 25-37, wherein

a) is administered at the same time as or after b).

Embodiment 39 is a combination according to Embodiments 25-36, wherein a) and b) are administered to the cancer patient in a priming administration followed by one or more boosting administrations of a) and b) to the cancer patient.

Embodiment 40 is a combination according to Embodiments 25-39, wherein the cancer patient is suffering from and/or is diagnosed with a cancer selected from the group consisting of: breast cancer, lung cancer, head and neck cancer, thyroid, melanoma, gastric cancer, bladder cancer, kidney cancer, liver cancer, melanoma, pancreatic cancer, prostate cancer, ovarian cancer, or colorectal cancer.

Embodiment 41 is a combination according to Embodiment 40, wherein the breast cancer is a HER2 overexpressing breast cancer.

Embodiment 42 is a combination, combination for use, a method, and/or combination therapy according to Embodiments 1-40, wherein the cancer is a MUC-1 overexpressing cancer.

Embodiment 43 is a combination, combination for use, a method, and/or combination therapy according to Embodiments 1-40, wherein the cancer is a CEA overexpressing cancer.

Embodiment 44 is a combination, combination for use, a method, and/or combination therapy according to Embodiments 1-40, wherein the cancer is a PSA overexpressing cancer.

Embodiment 45 is a combination, combination for use, a method, and/or combination therapy according to Embodiments 1-40, wherein the cancer is a Brachyury overexpressing cancer.

Embodiment 46 is a combination for use, a method, and/or combination therapy of any one of Embodiments 1-17, wherein the cancer patient is suffering from and/or is diagnosed with a cancer selected from the group consisting of: breast cancer, lung cancer, melanoma, bladder cancer, prostate cancer, ovarian cancer, or colorectal cancer.

Embodiment 47 is a combination for use, a method, and or combination

therapy of any one of Embodiments 1-17, wherein the cancer patient is suffering from and/or is diagnosed with breast cancer.

Embodiment 48 is a combination for use, a method, and/or combination therapy of any one of Embodiments 1-17, wherein the cancer patient is suffering from and/or is diagnosed with colorectal cancer.

Embodiment 49 is a combination for use according to Embodiments 1-24, wherein the combination is a pharmaceutical combination.

The references included as part of the present disclosure are hereby incorporated by reference in their entirety, including the following: World Health Report (2013), World Health Organization; Torre (2012) CA: A Cancer Journal for Clinicians 65 doi 10.3322/caac.21262, “Global Cancer Statistics”; Ross (2003) Oncologist 8: 307-325, “The HER2/neu gene and protein in breast cancer 2003: biomarker and target of therapy”; Palena (2007) Clin. Cancer Res. 13: 2471-8, “The human T-box mesodermal transcription factor Brachyury is a candidate target for T-cell-mediated cancer immunotherapy”; Hynes and Lane (2005) Nat. Rev. Cancer 5: 341-54, “ERBB receptors and cancer: the complexity of targeted inhibitors”; Cho (2003) Nature 421: 756-60, “Structure of the extracellular region of HER2 alone and in complex with the Herceptin Fab”; Satyanarayanajois (2009) Chem. Biol. Drug Des. 74: doi 10.1111/j.1747-0285.2009.00855.x, “Design, Synthesis, and Docking Studies of Peptidomimetics based on HER2-Herceptin Binding Site with Potential Antiproliferative Activity Against Breast Cancer Cell Lines”; Franklin (2004) Cancer Cell 5: 317-28, “Insights into ErbB signaling from the structure of the ErbB2-pertuzumab complex”; Yang (2009), Targeting The Dimerization Of ERBB Receptor, All Theses and Dissertations (ETDs), Paper 391; Tan (2005) Cancer Res. 65: 1858-67, “ErbB2 promotes Src synthesis and stability: novel mechanisms of Src activation that confer breast cancer metastasis”; Roskoski (2014) Pharmacol. Res. 87: 42-59, “ErbB/HER protein-tyrosine kinases: Structures and small molecule inhibitors”; Roselli (2012) Clin. Cancer Res. 18: 3868-79, “Brachyury, a driver of the epithelial-mesenchymal transition, is overexpressed in human lung tumors: an opportunity for novel interventions against lung cancer”; Stoller and Epstein (2005) Hum. Mol. Gen. 14: 885-92, “Identification of a novel nuclear localization signal in Tbx1 that is deleted in DiGeorge syndrome patients harboring the 1223delC mutation”; Lauterbach (2013) Front. Immunol. 4: 251, “Genetic Adjuvantation of Recombinant MVA with CD40L Potentiates CD8 T Cell Mediated Immunity”; Guardino (2009) Cancer Res. 69 (24 Supp Abstract nr 5089), “Results of Two Phase I Clinical Trials of MVA-BN®-HER2 in HER2 Overexpressing Metastatic Breast Cancer Patients,” Heery et al. (2015) J. Immunother. Cancer 3 (Suppl. 2): P132, “Phase I, dose-escalation, clinical trial of MVA-Brachyury-TRICOM vaccine demonstrating safety and brachyury-specific T cell responses”; Brodowicz et al. (2001) Br. J. Cancer 85: 1764-70, “Anti-HER2/neu antibody induces apoptosis in HER2/neu overexpressing breast cancer cells independently from p53 status”; Stackaruk et al. (2013) Expert Rev. Vaccines 12: 875-84, “Type I interferon regulation of natural killer cell function in primary and secondary infections”; Muller et al. (2017) Front. Immunol., “Type I Interferons and Natural Killer Cell Regulation in Cancer”; Yamashita et al. (2016) Scientific Reports 6: (article number 19772), “A novel method for evalulating antibody dependent cell-mediated cytotoxicity by flow cytometry using human peripheral blood mononuclear cells,” Broussas et al. (2013) Methods Mol. Biol. 988: 305-317, “Evaluation of antibody-dependent cell cytotoxicity using lactate dehydrogenase (LDH) measurement”; Tay et al. (2016) Human Vaccines and Immunotherapeutics 12: 2790-96, “TriKEs and BiKEs join CARs on the cancer immunotherapy highway”; Kono et al. (2004) Clin. Cancer Res. 10: 2538-44, “Trastuzumab (Herceptin) Enhances Class I-Restricted Antigen Presentation Recognized by Her2/neu Specific T Cytotoxic Lymphocytes.”

### EXAMPLES

The following examples illustrate the invention but should not be construed as in any way limiting the scope of the claims.

### Example 1: Intravenous Administration of Recombinant MVA Results in Stronger Activation of NK Cells

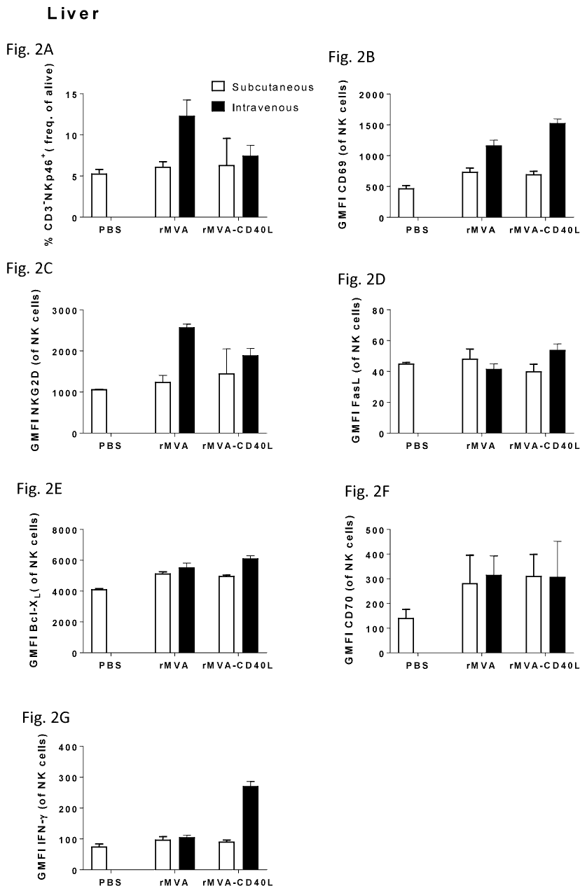

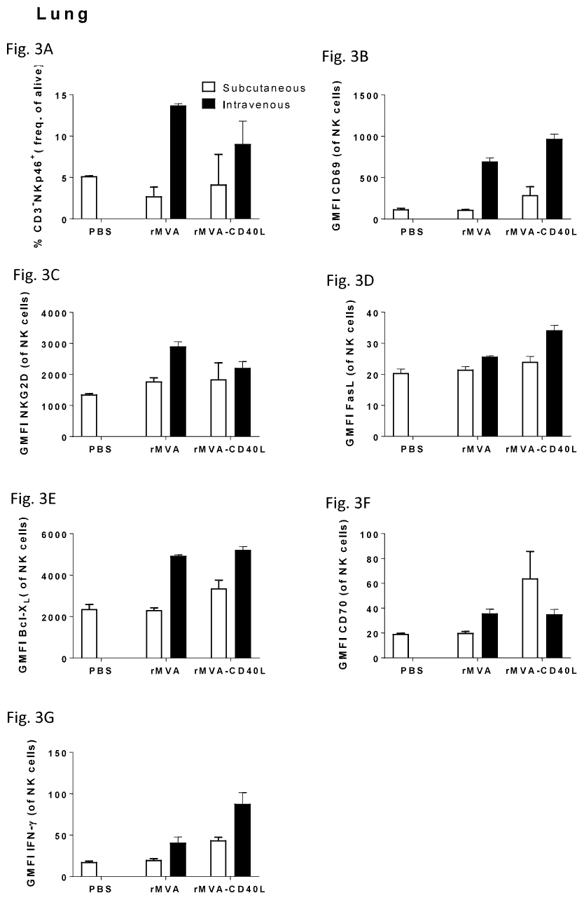

C57BL/6 mice were immunized subcutaneously (SC) or intravenously (IV) with 5×10⁷ TCID50 MVA-OVA (shown as rMVA) or MVA-OVA-CD40L (shown as IMVA-CD40L). PBS was injected SC. One day later, NK cell frequencies and protein expression (shown as Geometric Mean Fluorescence Intensity (GMFI)) were assessed using flow cytometry in the spleen (shown in FIGS. 1A-1G), in the liver (shown in FIGS. 2A-2G), and in the lung (shown in FIGS. 3A-3G) by staining for A) NKp46⁺ CD3³¹ cells; B) CD69; C) NKG2D; D) FasL; E); Bcl-X_{L}; F), CD70; and G) IFN-γ.

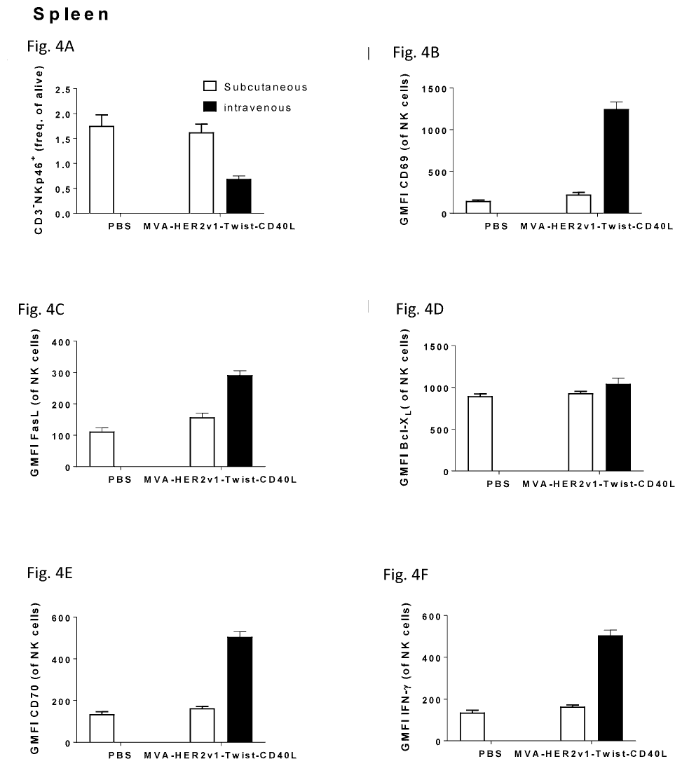

Additionally, C57BL/6 mice were immunized subcutaneously (SC) or intravenously (IV) with 5×10⁷ TCID₅₀ of a recombinant MVA encoding HER2v1, TWIST, and CD40L antigens (shown as MVA-HER2v1-Twist-CD40L). PBS was injected subcutaneously (SC). One day later, NK cell frequencies and protein expression (shown as Geometric Mean Fluorescence Intensity (GMFI)) were assessed using flow cytometry in the spleen (shown FIGS. 4A-4F), in the liver (shown in FIGS. 5A-5F), and in the lung (shown in FIGS. 6A-6F) by staining for A) NKp46⁺ CD3⁻ cells; B) CD69; C) FasL; D); Bcl-X_{L}; E), CD70; and F) IFN-γ.

Shown in the Figures, splenic NK cell frequencies dropped or were maintained after rMVA, rMVA-CD40L, and MVA-HER2v1-Twist-CD40L injection regardless of the application route. In A) IV rMVA application increased NK cell frequencies in liver and lung as compared to SC application. In B) CD69 is a stimulatory receptor for NK cells (Borrego et al., Immunology 1999) and is strongly upregulated after IV but not SC injection of rMVA, rMVA-CD40L, and MVA-HER2v1-Twist-CD40L. The highest CD69 expression was induced by rMVA-CD40L IV application. In FIGS. 1C, 2C, and 3C, the activating C-type lectin-like receptor NKG2D is upregulated on NK cells after rMVA and rMVA-CD40L immunization as compared to PBS treatment. In FIGS. 1D, 2D, and 3D and FIGS. 4C, 5C, and 6C, the apoptosis-inducing factor FasL (CD95L) is upregulated on NK cells after rMVA and rMVA-CD40L immunization as compared to PBS treatment. In FIGS. 1D, 2D, and 3D and FIGS. 4C, 5C, and 6C spleen and lung, FasL expression was highest after IV rMVA-CD40L and MVA-HER2v1-Twist-CD40Linjection. In FIGS. 1E, 2E, and 3E and 4D, 5D, and 6D IV rMVA-CD40L and MVA-HER2v1-Twist-CD40L immunization also lead to a higher expression of the anti-apoptotic Bcl family member Bcl-XL as compared to SC immunization. In FIGS. 1F, 2F, 3F and 4E, 5E, and 6E upregulation of the co-stimulatory molecule CD70, a member of the tumor necrosis factor (TNF) superfamily, is induced by IV injection of rMVA, rMVA-CD40L, and MVA-HER2v1-Twist-CD40L, especially on splenic NK cells. In FIGS. 1G, 2G, and 3G and 4F, 5F, and 6F importantly, the effector cytokine IFN-γ is most strongly expressed after IV rMVA-CD40L or IV MVA-HER2v1-Twist-CD40L immunization in spleen, lung and liver. These data show that IV immunization with either rMVA-CD40L or MVA-HER2v1-Twist-CD40L but not SC immunization leads to a strong, systemic NK cell activation.

### Example 2: Intravenous Administration of Recombinant MVA-CD40L Results in Stronger Systemic Activation of NK Cells

C57BL/6 mice were immunized IV with 5×10⁷ TCID₅₀ MVA-OVA (rMVA), MVA-OVA-CD40L (rMVA-CD40L), or PBS. Six hours after injection, serum cytokine levels (FIG. 7A) IFN-γ, (FIG. 7B) IL-12p70, and (FIG. 7C) CD69⁺ granzyme B⁺ were quantified by a bead assay (Luminex) and flow cytometry, as shown in FIGS. 7A-7F. The NK cell activating cytokine IL-12p70 was only detectable after rMVA-CD40L immunization. The concentration of IFN-γ was higher after rMVA-CD40L as compared to rMVA immunization. The increased serum levels of IFN-γ are in line with higher GMFI IFN-γ of NK cells (compared to FIG. 1G) and higher frequencies of spleen CD69″ Granzyme B⁺ NK cells 48 hours after rMVA-CD40L immunization.

Similar responses were seen in NHPs (Macaca fascicularis) after IV injection of MVA-MARV-GP-huCD40L, namely higher serum concentrations of IFN-γ(FIG. 7D) and IL-12p40/70 (FIG. 7E) as well as more proliferating (Ki67⁺) NK cells (FIG. 7F) as compared to MVA-MARV-GP. These data, shown in FIG. 7D-7E, demonstrate that CD40L-encoding MVA vaccines have comparable immunological properties in mice and NHPs.

### Example 3: Intravenous Administration of Recombinant MVA Induces strong CD8 T Cell Responses

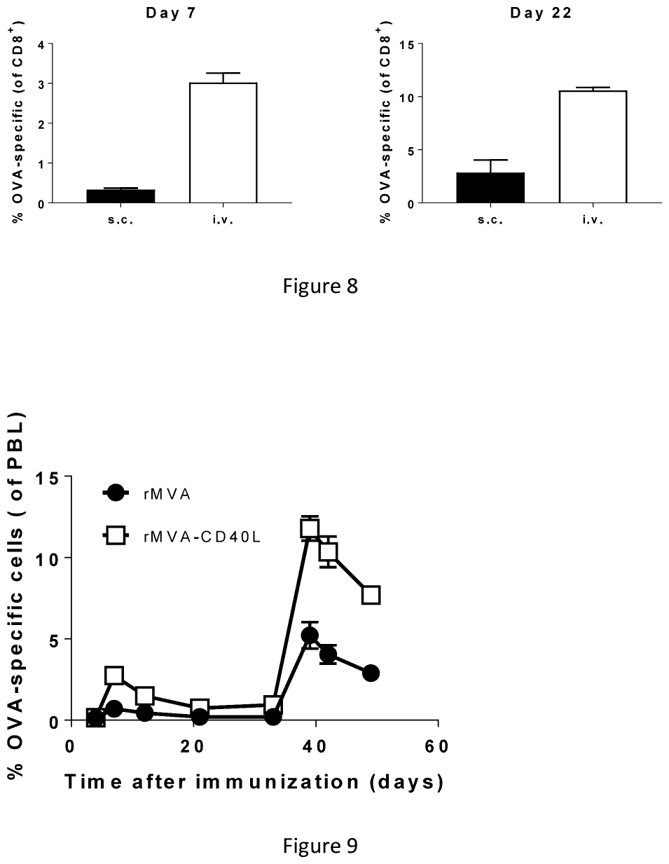

C57BL/6 mice were immunized intravenously (IV) or subcutaneously (SC) with 5×10⁷ TCID50 MVA-OVA on days 0 and 16. On days 7 and 22, OVA-specific CD8 T cell responses in the blood were assessed by flow cytometry after staining with H-2Kb/OVA₂₅₇₋₂₆₄ dextramers. Shown in FIG. 8, on day 7 the frequency of OVA-specific CD8 T-cells was 9-fold higher as compared to SC injections. On day 22, OVA-specific T-cells were 4-fold higher than after SC injection.

### Example 4: Intravenous Administration of Recombinant MVA-CD40L Further Enhances CD8 T Cell Responses

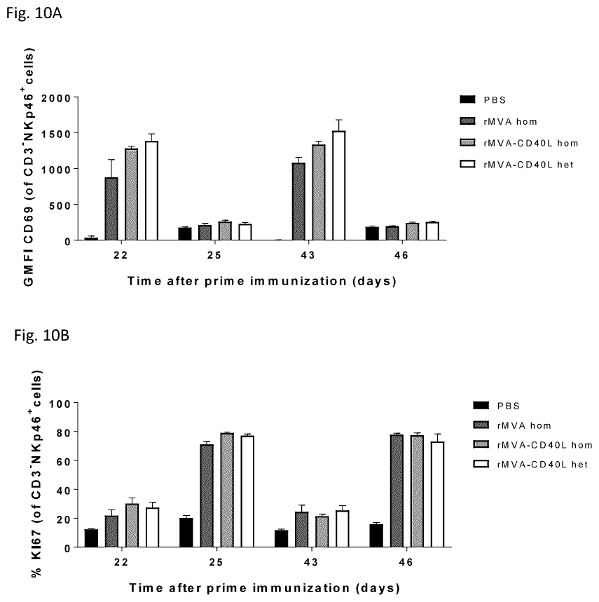

Shown in FIG. 9, C57BL/6 mice were immunized intravenously with 5×10⁷ TCID 50 MVA-OVA or MVA-OVA-CD40L on days 0 and 35. OVA-specific CD8 T cell responses in the blood were assessed by flow cytometry after staining with H-2Kb/OVA₂₅₇₋₂₆₄ dextramers. At the peak of the primary (day 7) and secondary (day 39) response, the frequency of OVA-specific CD8 T cells was enhanced 4-fold and 2-fold, respectively after MVA-OVA-CD40L compared to MVA-OVA immunization (Lauterbach et al. (2013), op. cit.).

### Example 5: Prime-Boost Immunization Shows Repeated NK Cell Activation and Proliferation

TABLE 1
Examples 5-7 IV immunization scheme

| groups | prime day 0 | boost day 21 | boost day 42 |
|---|---|---|---|
| PBS | PBS | PBS | PBS |
| rMVA hom | rMVA | rMVA | rMVA |
| rMVA-CD40L hom | rMVA-CD40L | rMVA-CD40L | rMVA-CD40L |
| rMVA-CD40L het | rMVA-CD40L | rMVA | rMVA |

C57BL/6 mice were immunized IV as shown in Table 1 (recombinant MVA dosages were at 5×10⁷ TCID₅₀). Note “hom” refers to “homologous prime-boost,” and “het” to “heterologous prime-boost.” NK cells (CD3− NKp46+) were analyzed in the blood by flow cytometry one and four days after second and third immunization. Shown in FIGS. 10A and 10B are the GMFI CD69 (FIG. 10A) and the frequency of Ki67+ NK cells (FIG. 10B). FIGS. 10A and 10B illustrate that NK cells are activated by each immunization despite the presence of anti-vector immunity. This unexpected finding supports combination of antibody therapy with boost immunizations that would activate NK cells. Thus, when cancer patients are treated multiple times with recombinant MVA and mount anti-vector responses, NK cell activation is not impaired. In contrast, each treatment leads to de novo NK cell activation.

### Example 6: Prime-Boost Immunization Shows Stronger Induction of CD4 T Helper Cells

C57BL/6 mice were immunized as shown in Table 1 (recombinant MVA dosages were at 5×10⁷ TCID₅₀). Serum cytokine levels were quantified at 6 hours post immunization by a multiplex bead assay (Luminex). Shown are the results from the expression of the named cytokines. FIG. 11A) IL-6; FIG. 11B) CXCL10; FIG. 11C) IFN-α; FIG. 11D) IL-22; FIG. 11E) IFN-γ; FIG. 11F) CXCL1; FIG. 11G) CCL4; FIG. 11H) CCL7; FIG. 11I) CCL2; FIG. 11J) CCL5; FIG. 11K) TNF-α; FIG. 11L) IL-12p70; and FIG. 11M) IL-18.

Shown in FIGS. 11A-11M, rMVA-CD40L hom-treated mice (i.e., mice treated with a homologous prime-boost) had a similar cytokine profile as mice primed with rMVA and boosted with rMVA-CD40L (rMVA-CD40L het). rMVA hom-treated mice displayed lower levels of IL-6, IL12p70, IL-22, IFN-α, TNF-α, CCL2, CCL5 and CXCL1 after the first and second immunization compared to mice primed with rMVA-CD40L. A cytokine absent after the prime but highly produced after second and third immunization was IL-22. IL-22 is largely produced by effector T helper cells and subpopulations of innate lymphocyte cells. The higher expression of IL-22 in rMVA-CD40L het or rMVA-CD40L hom-treated mice thus indicates stronger induction CD4 T helper responses by rMVA-CD40L immunization. Overall, IV rMVA and rMVA-CD40L immunization induced high systemic cytokine responses that are highest in mice primed with rMVA-CD40L.

### Example 7: Prime-Boost Immunization Shows Stronger Effector Memory CD8 and CD4 T Cell Responses

C57BL/6 mice were immunized IV as shown in Table 1. The results are shown in FIG. 12A and 12B. Phenotypically, effector and effector memory T cells can be identified by the expression of CD44 and the lack of surface CD62L. Monitoring CD44+ CD62L− CD8 (FIG. 12A) and CD4 (FIG. 12B) T cells in the blood demonstrated that repeated IV immunization induces expansion of effector and effector memory T cells. Interestingly, mice that received either rMVA-CD40L hom or rMVA-CD40L het had about 2.5 fold more circulating effector CD4 T cells than mice primed with rMVA (FIG. 12B, day 25). This indicates that systemic priming with rMVA-CD40L induces stronger CD4 T cell responses than rMVA.

### Example 8: Intravenous Administration of Recombinant MVA Results in Strong Anti-Tumor Effects in Treating Melanoma

B16.OVA tumors express the foreign model antigen ovalbumin (“OVA”). C57BL/6 mice bearing palpable B16.OVA tumors were primed (dotted line) either IV or SC with PBS, MVA-OVA (rMVA) or MVA-OVA-CD40L (rMVA-CD40L) (recombinant MVA dosages were at 5×10⁷ TCID₅₀). At 7 and 14 days after prime immunization, the mice received subsequent boosts with FPV-OVA at 5×10⁷ TCID₅₀ (dashed lines). Tumor growth was measured at regular intervals. Shown in FIG. 13A and 13B are tumor mean volume (FIG. 13A) and survival of tumor-bearing mice by day 45 after tumor inoculation (FIG. 13B). Thus, priming of B16.OVA tumor bearing mice IV with rMVA-CD40L provides a stronger anti-tumor effect as compared to both SC rMVA-CD40L or SC or IV rMVA.

### Example 9: A Single Intravenous Administration of Recombinant MVA Results in Strong Anti-Tumor Effects

TABLE 2
Vaccination scheme corresponding to Example 9.

| | Day 8 | Day 15 |
|---|---|---|
| Group | Prime | Boost |
| PBS | PBS | none |
| rMVA | rMVA | none |
| rMVA 2x | rMVA | rMVA |
| rMVA→MVA-CD40L | rMVA | rMVA-CD40L |
| rMVA→CD40L | rMVA-CD40L | none |
| rMVA-CD40L→rMVA | rMVA-CD40L | rMVA |
| rMVA-CD40L 2x | rMVA-CD40L | rMVA-CD40L |
| rMVA-CD40L→rFPV | rMVA-CD40L | rFPV |

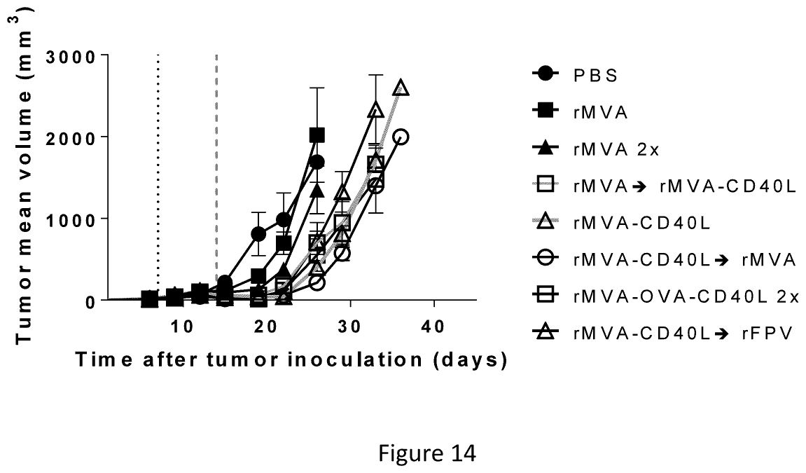

C57BL/6 mice bearing palpable B16.OVA tumors were IV vaccinated as shown in Table 2. Tumor growth was measured at regular intervals. Shown in FIG. 14 is tumor mean volume. The results indicate that a single therapeutic immunization with rMVA-CD40L is as strong as homologous or heterologous prime/boost immunizations. Importantly, these data highlight the potent anti-tumor activity of rMVA-CD40L.

### Example 10: Intravenous Administration of Recombinant MVA-CD40L Increased T-cell Infiltration in the Tumor Microenvironment

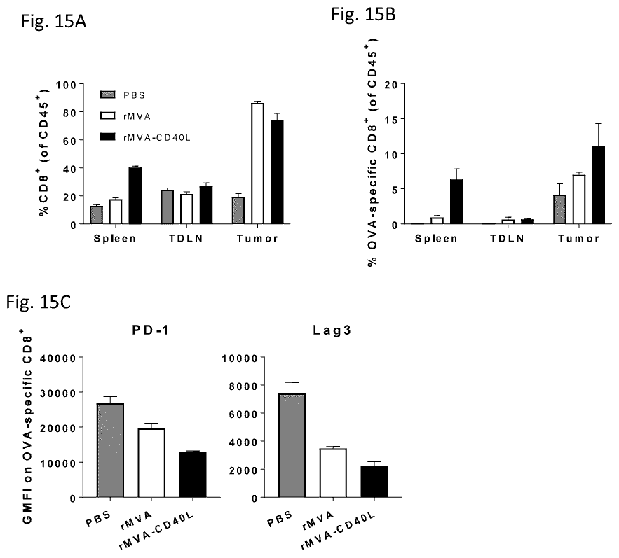

C57BL/6 mice bearing palpable B16.OVA tumors were immunized intravenously with PBS, rMVA (MVA-OVA) or rMVA-CD40L (MVA-OVA-CD40L) (recombinant MVA dosages were at 5×10⁷ TCID₅₀). After 7 days, mice were sacrificed. As shown in FIG. 15A and 15B, the frequency and distribution of CD8⁺ T cells and OVA257-264-specific CD8+ T cells was analyzed among leukocytes in spleen, tumor-draining lymph nodes (TDLN) and tumor tissues. Shown in FIG. 15C, geometric mean fluorescence intensity (GMFI) of PD-1 and Lag3 on tumor-infiltrating OVA₂₅₇₋₂₆₄-specific CD8⁺ T cells was analyzed.

Taken together, these data show that rMVA-CD40L immunization leads to a more pronounced infiltration of TAA-specific CD8 T cells into the tumor microenvironment (TME) compared to PBS and that rMVA-CD40L immunization leads a reduction in PD-1 and Lag3 expression.

### Example 11: Intravenous Administration of Recombinant MVA-CD40L Decreased Levels of Treg in Tumor Microenvironment

Purified OVA-specific TCR-transgenic CD8 T cells (OT-I) were intravenously transferred into B16.OVA tumor bearers when tumors were palpable. When tumors reached at least 60 mm3 in volume animals were immunized with MVA-BN, MVA-OVA (rMVA), or MVA-OVA-CD40L (rMVA-CD40L) (recombinant MVA dosages were at 5×10⁷ TCID₅₀). After 17 days mice were sacrificed and analyzed for frequency of Foxp3+ CD4+ Treg among CD4+ T cells in tumor tissues. The results are shown in FIG. 16. Taken together, these data show that even after prolonged exposure to the TME, a single immunization with rMVA-CD40L leads to a reduction of Treg compared to control treatment (MVA-BN without encoded TAA) or rMVA immunization.

### Example 12: Intravenous Administration of Recombinant MVA-CD40L Increased Longevity of T-cell Infiltration of Tumor Microenvironment

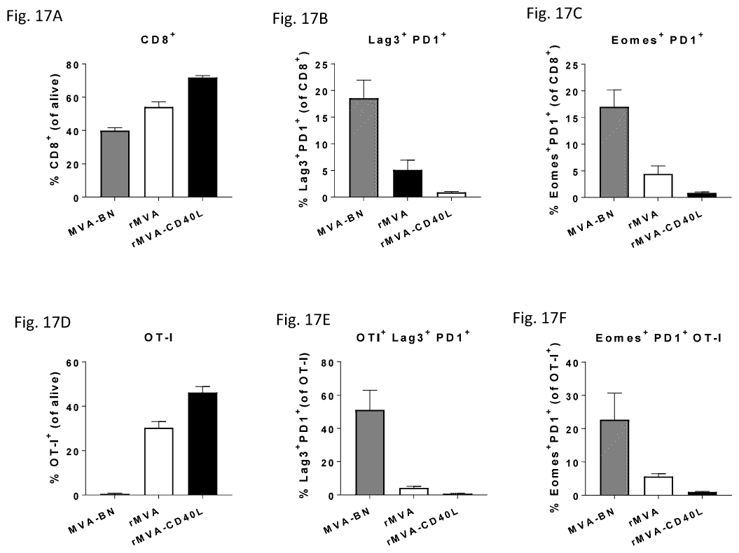

TCR-transgenic OVA-specific CD8 T cells (OT-I) were intravenously transferred into B16.OVA tumor bearers when tumors were palpable. When tumors reached at least 60 mm³ in volume animals were immunized with MVA-BN, MVA-OVA (rMVA), or MVA-OVA-CD40L (rMVA-CD40L) (recombinant MVA dosages were at 5×10⁷ TCID₅₀). After 17 days, mice were sacrificed and analyzed for (FIG. 17A) Frequency of CD8⁺ T cells among leukocytes in tumor tissues; (FIG. 17B) Frequency of Lag3⁺ PD1⁺ within CD8⁺ T cells; (FIG. 17C) Frequency of Eomes⁺ PD1⁺ T cells within CD8⁺ T cells; (FIG. 17D) Presence of OT-I-transgenic CD8⁺ T cells within the TME upon immunization; and (FIG. 17E) Frequency of Lag3⁺ PD1⁺ exhausted T cells within OT-I⁺ CD8⁺ T cells; and (FIG. 17F) Frequency of Eomes⁺ PD1⁺ exhausted T cells within OT-I⁺ CD8⁺ T cells. The results are shown in FIG. 17A-17F. These data indicate that TAA-specific CD8 T cells that are recruited into the TME upon rMVA-CD40L immunization show less signs of immune exhaustion than after control treatment (MVA-BN without encoded TAA) or rMVA immunization even after prolonged exposure to the TME.

Shown in FIGS. 15A-15C and 17A-17F, the expression of PD1⁺ and Lag3⁺ decreased upon intravenous administration with rMVA with the expression of PD1⁺ and Lag3⁺ being further decreased upon intravenous administration with rMVA-CD40L.

### Example 13: Construction of Recombinant MVA viruses MVA-mBN445, MVA-mBN451, MVA-mBNbc197, MVA-mBNbc195, MVA-mBNbc388, MVA-mBN bc389, and MVA-mBN484

Generation of recombinant MVA viruses that embody elements of the combination therapy (e.g., MVA-mBN445, MVA-mBN451 and MVA-mBN484) was done by insertion of the indicated transgenes with their promoters into the vector MVA-BN. Transgenes were inserted using recombination plasmids containing the transgenes and a selection cassette, as well as sequences homologous to the targeted loci within MVA-BN. Homologous recombination between the viral genome and the recombination plasmid was achieved by transfection of the recombination plasmid into MVA-BN infected CEF cells. The selection cassette was then deleted during a second step with help of a plasmid expressing CRE-recombinase, which specifically targets loxP sites flanking the selection cassette, therefore excising the intervening sequence.

For construction of mBNbc346 the recombination plasmid included the transgenes AH1A5, p15e, and TRP2 each preceded by a promoter sequence, as well as sequences which are identical to the targeted insertion site within MVA-BN to allow for homologous recombination into the viral genome.

For construction of mBNbc354 the recombination plasmid included the transgenes AH1A5, p15e, and TRP2, and CD40L, each preceded by a promoter sequence, as well as sequences which are identical to the targeted insertion site within MVA-BN to allow for homologous recombination into the viral genome.

For the construction of mBN451, the recombination plasmid included two transgenes HER2v1 and Brachyury (SEQ ID NO: 1 and SEQ ID NO: 5, respectively), each preceded by a promoter sequence, as well as sequences which are identical to the targeted insertion site within MVA-BN to allow for homologous recombination into the viral genome. The HER2 and Brachyury coding sequences (or nucleotide sequences) are SEQ ID NO: 2 and SEQ ID NO: 6, respectively.

For the construction of mBN445 the recombination plasmid included the three transgenes HER2v1, Brachyury, and CD40L (SEQ ID NO: 1, SEQ ID NO: 5, and SEQ ID NO: 13, respectively), each preceded by a promoter sequence, as well as sequences which are identical to the targeted insertion site within MVA-BN to allow for homologous recombination into the viral genome. The HER2, Brachyury, and CD40L coding sequences (or nucleotide sequences) are SEQ ID NO: 2, SEQ ID NO: 6, and SEQ ID NO: 14, respectively.

For construction of mBNbc388 the recombination plasmid included the three transgenes HER2v1, Twist, and CD40L (amino acid sequences SEQ ID NO: 1, SEQ ID NO: 15, and SEQ ID NO: 17, respectively), each preceded by a promoter sequence, as well as sequences which are identical to the targeted insertion site within MVA-BN to allow for homologous recombination into the viral genome. The HER2v1, Twist, and CD40L coding sequences (or nucleotide sequences) are SEQ ID NO: 2, SEQ ID NO: 16, and SEQ ID NO: 18, respectively.

For construction of mBNbc389 the recombination plasmid included the two transgenes HER2v1 and Twist (amino acid sequences SEQ ID NO: 1, SEQ ID NO: 15, respectively), each preceded by a promoter sequence, as well as sequences which are identical to the targeted insertion site within MVA-BN to allow for homologous recombination into the viral genome. The HER2v1, Twist, and CD40L coding sequences (or nucleotide sequences) are SEQ ID NO: 2 and SEQ ID NO: 16 respectively.

For construction of mBN484 the recombination plasmid included the three transgenes HER2v2, Brachyury, and CD40L (amino acid sequences SEQ ID NO: 3, SEQ ID NO: 5, and SEQ ID NO: 13, respectively), each preceded by a promoter sequence, as well as sequences which are identical to the targeted insertion site within MVA-BN to allow for homologous recombination into the viral genome. The HER2v2, Twist, and CD40L coding sequences (or nucleotide sequences) are SEQ ID NO: 4, SEQ ID NO: 6, and SEQ ID NO: 14, respectively.

For generation of the above described mBN MVAs, (e.g, .mBN445, mBN451, and mBN484), CEF cell cultures were each inoculated with MVA-BN and transfected each with the corresponding recombination plasmid. In turn, samples from these cell cultures were inoculated into CEF cultures in medium containing drugs inducing selective pressure, and fluorescence-expressing viral clones were isolated by plaque purification. Loss of the fluorescent protein-containing selection cassette from these viral clones was mediated in a second step by CRE-mediated recombination involving two loxP sites flanking the selection cassette in each construct. After the second recombination step only the transgene sequences (e.g., HER2, Brachyury, and/or CD40L) with their promoters inserted in the targeted loci of MVA-BN were retained. Stocks of plaque-purified virus lacking the selection cassette were prepared.

Expression of the identified transgenes was subsequently demonstrated in cells inoculated with the described construct (See, e.g., FIG. 17A-17F).

Generation of mBNbc388, mBNbc389, mBNbc346, and mBNbc354 was carried out by using a cloned version of MVA-BN in a bacterial artificial chromosome (BAC). Recombination plasmids each containing the different transgenes for mBNbc388 and mBNbc389, and mBNbc346 and mBNbc354 were used. The plasmids included sequences that are also present in MVA and therefore allow for specific targeting of the integration site. Nucleotide sequences encoding the AH1A5, p15e, OVA, Her2 v1, Twist, TRP2, and/or CD40L antigens were present between the MVA sequences that allow for recombination into the MVA viral genome. Thus, a plasmid was constructed for each construct that contained the AH1A5, p15e, OVA, HER2v1, Twist, TRP2 and/or CD40L coding sequences, each downstream of a promoter. Briefly, infectious viruses were reconstituted from BACs by transfecting BAC DNA into BHK-21 cells and superinfecting them with Shope fibroma virus as a helper virus. After three additional passages on CEF cell cultures, helper-virus free MVA-mBNbc388 and MVA-mBNbc389 were obtained. An exemplary MVA generation is also found in Baur et al. (2010) Virol. 84: 8743-52, “Immediate-early expression of a recombinant antigen by modified vaccinia virus Ankara breaks the immunodominance of strong vector-specific B8R antigen in acute and memory CD8 T-cell responses.”

### Example 14: Heterologous Expression of MVA-HER2-Brachyury-CD40L

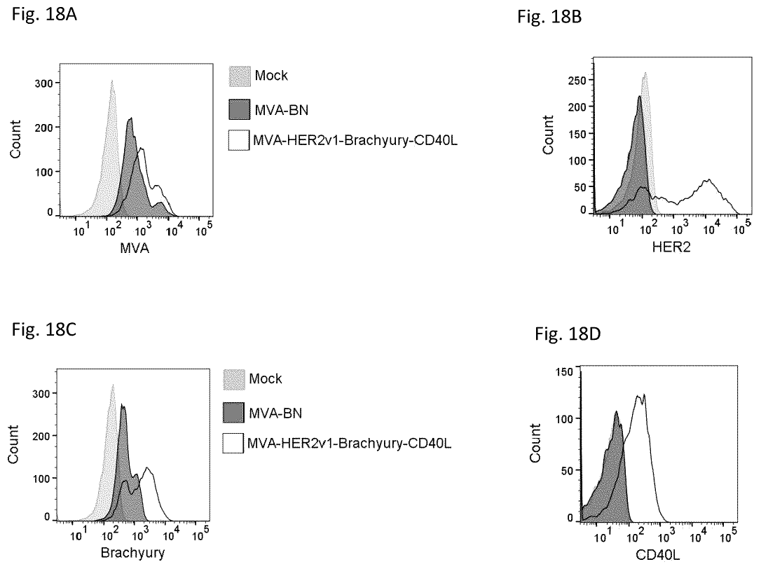

HeLa cells were left untreated (mock) or infected with MVA-BN or MVA-HER2v1-Brachyury-CD40L (MVA-mBN445). After overnight culture, cells were stained with anti-HER2-APC (clone 24D2), anti-Brachyury (rabbit polyclonal)+anti-rabbit IgG-PE and anti-CD40L-APC (clone TRAP1). Shown in FIG. 18A-18D, flow cytometric analysis revealed expression of all three transgenes.

### Example 15: Enhanced Activation of Human DCs by MVA-HER2-Brachyury-CD40L

Monocyte-derived dendritic cells (DCs) were generated after enrichment of CD14+ monocytes from human PBMCs and cultured for 7 days in the presence of GM-CSF and IL-4 according to protocol (Miltenyi, MO-DC generation tool box). DCs were stimulated with MVA-HER2v1-Brachyury or MVA-HER2-Brachyury-CD40L. Shown is expression of (FIG. 19A) CD40L, (FIG. 19B) CD86, and (FIG. 19C) and MHC class II was analyzed by flow cytometry. Shown in (FIG. 19D), the concentration of IL-12p70 in the supernatant was quantified by Luminex after over-night culture.

This experiment demonstrates that rMVA-HER2v1-Brachyury-CD40L stimulates human DCs, inducing their activation and thus enhancing their capability to present antigens. The production of the Th1 polarizing and NK cell activating cytokine IL-12p70 by stimulated human DCs indicates that MVA-HER2v1-Brachyury-CD40L activates human DCs towards a pro-inflammatory phenotype.

### Example 16: Intravenous Administration of MVA-HER2-Twist-CD40L (mBNbc388) Enhances Infiltration of HER2 Specific CD8+ T Cells Into Tumors

Balb/c mice bearing CT26.HER2 tumors received intravenously either PBS or 5×10⁷ TCID₅₀ MVA-HER2v1-Twist-CD40L. Seven days later, mice were sacrificed, spleen and tumor-infiltrating CD8+ T cells isolated by magnetic cell sorting and cultured in the presence of HER2 peptide-loaded dendritic cells for 5 hours. Graph shows percentage of CD44+ IFNγ+ cells among CD8+ T cells. Results are shown as Mean±SEM. The results, illustrated in FIG. 20, demonstrate that the various embodiments of the present invention are tumor specific.

### Example 17: Increased Overall Survival and Tumor Reduction in IV Administration of rMVA-CD40L Combined With PD-1 Checkpoint Antagonist Blockade

TABLE 3
Vaccination scheme.

| | i.v. immunization | i.p. injection | | | |
|---|---|---|---|---|---|
| groups | day 0 | day 0 | day 3 | day 7 | day 10 |
| PBS | — | PBS | PBS | PBS | PBS |
| rMVA-CD40L | rMVA-CD40L | — | — | — | — |
| anti-PD-1 | — | anti-PD-1 | anti-PD-1 | anti-PD-1 | anti-PD-1 |
| rMVA-CD40L + anti-PD-1 | rMVA-CD40L | anti-PD-1 | anti-PD-1 | anti-PD-1 | anti-PD-1 |

C57BL/6 mice bearing 90 mm³ MC38 colon cancer were immunized IV with 5×10⁷ TCID₅₀ MVA-AH1A5-p15e-TRP2 -CD40L (shown in FIG. 20 as rMVA-p15e-CD40L). Immunization was subsequently followed by administration of 10 mg/kg PD-1 antibody or PBS on the same day followed by three additional antibody administrations within two weeks after immunization, as described in Table 3. Tumor growth was measured at regular intervals. Shown in FIG. 21A and 21B are the tumor mean volume (FIG. 21A) and tumor-free survival (FIG. 21B). These data indicate that PD-1 checkpoint blockade enhances antitumor effects exerted by single therapeutic immunization with a recombinant MVA encoding a tumor-associated antigen and CD40L, hence inducing tumor rejection in a colon cancer model.

### Example 18: Increased Overall Survival and Tumor Reduction in IV Administration of rMVA-HER2-Twist-CD40L Combined With Anti-PD-1 Checkpoint Blockade in a HER2 Expressing Colon Carcinoma

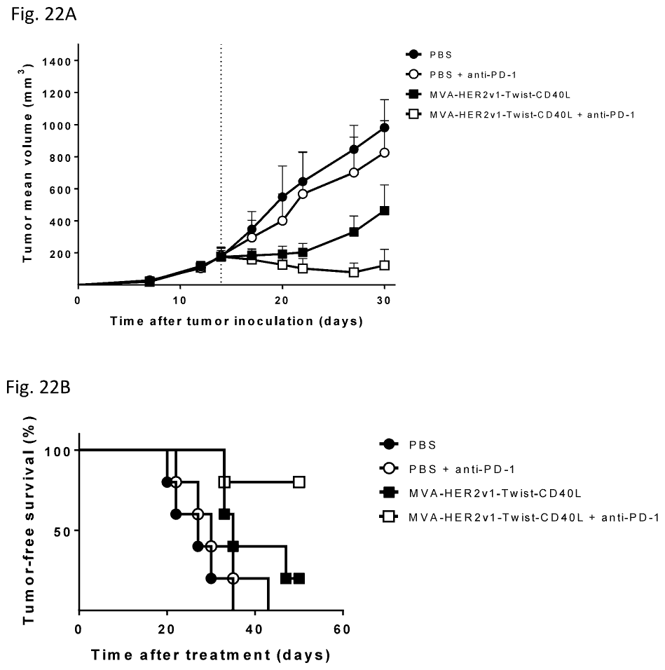

C57BL/6 mice bearing 85 mm3 MC38.HER2 colon cancers were immunized IV either with MVA-HER2v1-Twist-CD40L, or received PBS. Immunization was subsequently followed by a PD-1 antibody administration. Tumor growth was measured at regular intervals. Shown in FIG. 22 are the tumor mean volume (A) and tumor-free survival (B). These data indicate that PD-1 checkpoint blockade enhances antitumor effects exerted by single therapeutic immunization with rMVA-HER2v1-Twist-CD40L, hence inducing tumor rejection in a HER2-expressing colon cancer model.

Shown in both FIGS. 21A, 21B, 22A, and 22B, surprisingly, when adding the checkpoint antagonist PD-1 to the recombinant MVA encoding a TAA and CD40L there was an increased anti-tumor effect. Shown in FIGS. 15A-15C and 17A-17F, the expression of PD1 and Lag3 decreased upon intravenous administration with rMVA and expression further decreased upon intravenous administration with rMVA-CD40L.

Because the mouse homolog of Brachyury is neither highly expressed in normal mouse tissues nor predominantly expressed in mouse tumor tissues, the efficacy of Brachyury as a target for an active immunotherapy cannot be studied effectively in a mouse model system (see WO 2014/043535, which is incorporated by reference herein). Twist, the mouse homolog of the Human Brachyury is used in mouse models is a predictive model for Brachyury function in humans. This was demonstrated in WO 2014/043535. Like Brachyury, the mouse homolog of the EMT regulator Twist both promotes the EMT during development by down-regulating E-cadherin-mediated cell-cell adhesion and up-regulating mesenchymal markers and is predominantly expressed in mouse tumor tissue (see, e.g., FIG. 5 and Example 8 of WO 2014/043535). Therefore, the study of a Twist-specific cancer vaccine in mice is very likely to have strong predictive value regarding the efficacy of a Brachyury-specific cancer vaccine in humans. Id.

### Example 19: Increased Overall Survival and Tumor Reduction in IV Administration of rMVA-CD40L Combined With -CTLA-4 Checkpoint Blockade

C57BL/6 mice bearing 85 mm3 MC38 colon cancer are immunized IV with MVA-AH1A5-p15e-TRP2-CD40L (rMVA-CD40L), or receive PBS. Immunization is subsequently followed by a CTLA-4 antibody administration. Tumor growth is measured at regular intervals.

### Example 20: Increased Overall Survival and Tumor Reduction in IV Administration of rMVA-CD40L Combined With Lag3 Checkpoint Blockade

C57BL/6 mice bearing 85 mm3 MC38 colon cancer are immunized IV with MVA-AH1A5-p15e-TRP2-CD40L (rMVA-CD40L), or receive PBS. Immunization is subsequently followed by a Lag3 antibody administration. Tumor growth is measured at regular intervals.

### Example 21: Increased Overall Survival and Tumor Reduction in IV Administration of rMVA-CD40L Combined With TIM-3 Checkpoint Blockade

C57BL/6 mice bearing 85 mm3 MC38 colon cancer are immunized IV with MVA-AH1A5-p15e-TRP2 -CD40L (rMVA-CD40L), or receive PBS. Immunization is subsequently followed by a Tim3 antibody administration. Tumor growth is measured at regular intervals.

### Example 22: Intravenous Administration of Recombinant MVA-CD401, Increased Longevity of T-Cell Infiltration of Tumor Microenvironment

TCR-transgenic OVA-specific CD8 T cells (OT-I) are intravenously transferred into B16.OVA tumor bearers when tumors were palpable. When tumors reach at least 60 mm³ in volume animals are immunized with MVA-BN, MVA-OVA (rMVA), or MVA-OVA-CD40L (rMVA-CD40L) (recombinant MVA dosages were at 5×10⁷ TCID50). After 17 days, mice are sacrificed and analyzed for frequency of Lag3⁺ within CD8⁺ T cells; and frequency of TIM3⁺ CD8⁺ T cells.

### Example 23: Antitumor Effect of Intravenous Injection of MVA Virus Encoding the Endogenous Retroviral Antigen Gp70 and CD40L on CT26.wt Tumors

Recombinant MVAs encoding the murine endogenous retroviral antigen (ERV) protein Gp70 (envelope protein of the murine leukemia virus) with or without the costimulatory molecule CD40L were generated. The anti-tumor potential of these constructs was evaluated using the CT26.wt colon carcinoma model. CT26.wt cells have been shown to express high levels of Gp70 (see Scrimieri (2013) Oncoimmunol. 2: e26889).

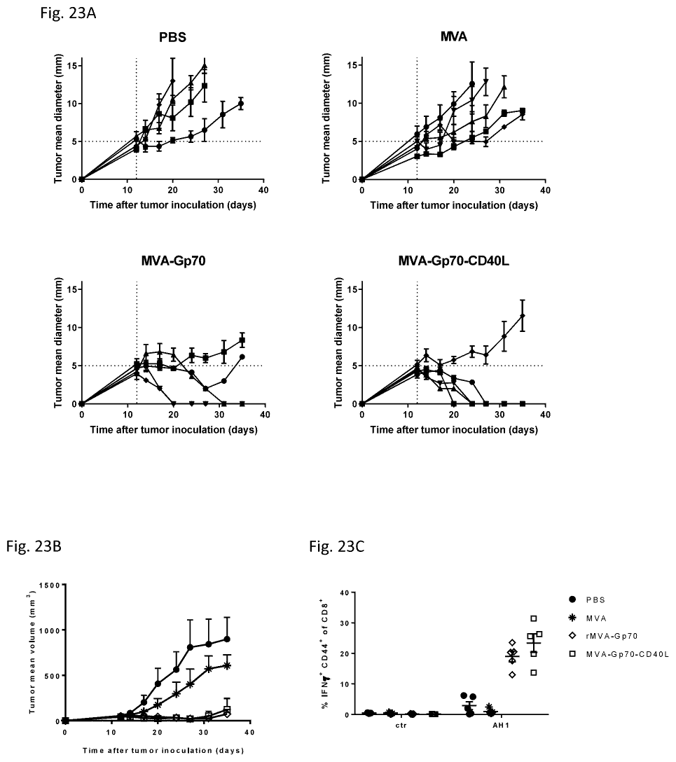

CT26.wt tumor-bearing mice were intravenously immunized when tumors were at least 5×5 mm. Treatment with MVA alone induced a mild growth delay in tumors, whereas treatment with MVA encoding GP70 resulted in complete rejection of ⅗ tumors (FIG. 13A and 13B). Treatment with MVA-Gp70-CD40L produced even more dramatic results of rejection of ⅘ tumors (FIG. 23A and 23B).

To determine whether the anti-tumor response was correlated with the induction of Gp70-specific T cells after immunization, a blood re-stimulation was performed using the H-2Kd-restricted gp70 epitope AH1. The results show a strong induction of Gp70-specific CD8 T cell responses in MVA-Gp70 and MVA-Gp70-CD40L treated mice (FIG. 23C).

### Example 24: Antitumor Effect of Intravenous Injection of MVA Virus Encoding the Endogenous Retroviral Antigen Gp70 and CD40L on B16.F10 Tumors

B16.F10 is a melanoma cell line derived from C57BL/6. Similar to CT26.wt cells, B16.F10 cells express high levels of Gp70 (see Scrimieri (2013) Oncoimmunol. 2: e26889). B16.F10 tumor-bearing mice were generated and used to further evaluate the antitumor effect of MVA encoding gp70 with or without CD40L (designated MVA-gp70-CD40L and MVA-gp70, respectively).

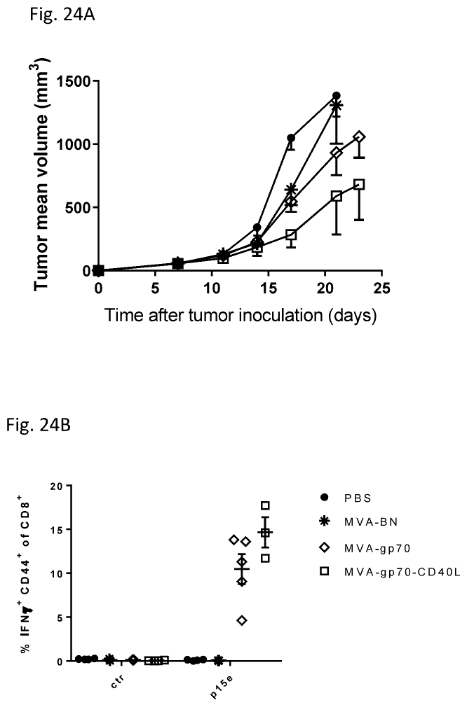

Treatment with MVA (“MVA-BN”) alone led to some tumor growth delay of B16.F10 tumors which was comparable to the effect of MVA-Gp70 (FIG. 24A), whereas MVA-Gp70-CD40L resulted in a stronger anti-tumor effect. Evaluation of CD8 T cell responses showed no significant increase of T cell responses was observed when CD40L was encoded (FIG. 24B).

It will be apparent that the precise details of the methods or compositions described herein may be varied or modified without departing from the spirit of the described invention. We claim all such modifications and variations that fall within the scope and spirit of the claims below.

We claim:

1. A combination for use in reducing tumor size and/or increasing survival in a cancer patient, the combination comprising:
a) a recombinant modified Vaccinia Ankara (MVA) virus comprising a first nucleic acid encoding a tumor-associated antigen (TAA) and a second nucleic acid encoding CD40 Ligand (CD40L), that when administered intravenously induces both an enhanced Natural Killer (NK) cell response and an enhanced T cell response as compared to an NK cell response and a T cell response induced by a non-intravenous administration of a recombinant MVA virus comprising a first nucleic acid encoding a TAA and a second nucleic acid encoding CD40 L; and
b) at least one antagonist or agonist of an immune checkpoint molecule;
wherein (a) and (b) are to be administered as a combination treatment; and wherein administration of a) and b) to the cancer patient reduces tumor size and/or increases the survival rate of the cancer patient as compared to a non-IV administration of a) or an administration of b) alone.

2. The combination for use of claim 1, wherein the antagonist or agonist of an immune checkpoint molecule comprises a CTLA-4 antagonist, a PD-1 antagonist, a PD-L1 antagonist, a LAG-3 antagonist, a TIM-3 antagonist, or an ICOS agonist.

3. The combination for use of claim 2, wherein the CTLA-4 antagonist is a CTLA-4 antibody, the PD-1 antagonist is a PD-1 antibody, the PD-L1 antagonist is a PD-L1 antibody, the LAG-3 antagonist is a LAG-3 antibody, the TIM-3 antagonist is a TIM-3 antibody, and the ICOS agonist is an ICOS antibody.

4. The combination for use of claim 1, wherein the (TAA is selected from the group consisting of: carcinoembryonic antigen (CEA), Mucin 1, cell surface associated (MUC-1), Prostatic Acid Phosphatase (PAP), Prostate Specific Antigen (PSA), human epidermal growth factor receptor 2 (HER2), survivin, tyrosine related protein 1 (TRP1), tyrosine related protein 2 (TRP2), Brachyury antigen, or combinations thereof.

5. The combination for use of claim 1, wherein the TAA is selected from the group consisting of: 5-α-reductase, α-fetoprotein (AFP), AM-1, APC, April, B melanoma antigen gene (BAGE), β-catenin, Bcl12, ber-ab1, Brachyury, CA-125, caspase-8 (CASP-8), Cathepsins, CD19, CD20, CD21/complement receptor 2 (CR2), CD22/BL-CAM, CD23/FcεRII, CD33, CD35/complement receptor 1 (CR1), CD44/PGP-1, CD45/leucocyte common antigen (LCA), CD46/membrane cofactor protein (MCP), CD52/CAMPATH-1, CD55/decay accelerating factor (DAF), CD59/protectin, CDC27, CDK4, carcinoembryonic antigen (CEA), c-myc, cyclooxygenase-2 (cox-2), deleted in colorectal cancer gene (DCC), DcR3, E6/E7, CGFR, EMBP, Dna78, farnesyl transferase, fibroblast growth factor-8a (FGF8a), fibroblast growth factor-8b (FGF8b), FLK-1/KDR, folic acid receptor, G250, G melanoma antigen gene family (GAGE-family), gastrin 17, gastrin-releasing hormone, ganglioside 2 (GD2)/ganglioside 3 (GD3)/ganglioside-monosialic acid-2 (GM2), gonadotropin releasing hormone (GnRH), UDP-GlcNAc:R1Man(α1-6)R2 [GlcNAc to Man(α1-6)] β1,6-N¬-acetylglucosaminyltransferase V (GnT V), GP1, gp100/Pme117, gp-100-in4, gp15, gp75/tyrosine-related protein-1 (gp75/TRP1), human chorionic gonadotropin (hCG), heparanase, HER2, human mammary tumor virus (HMTV), 70 kiloDalton heat-shock protein (“HSP70”), human telomerase reverse transcriptase (hTERT), insulin-like growth factor receptor-1 (IGFR-1), interleukin-13 receptor (IL-13R), inducible nitric oxide synthase (“iNOS”), Ki67, KIAA0205, K-ras, H-ras, N-ras, KSA, LKLR-FUT, melanoma antigen-encoding gene 1 (MAGE-1), melanoma antigen-encoding gene 2 (MAGE-2), melanoma antigen-encoding gene 3 (MAGE-3), melanoma antigen-encoding gene 4 (MAGE-4), mammaglobin, MAP17, Melan-A/melanoma antigen recognized by T-cells-1 (MART-1), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (PDGF), μPA, PRAME, probasin, progenipoietin, prostate-specific antigen (PSA), prostate-specific membrane antigen (PSMA), RAGE-1, Rb, RCAS1, SART-1, SSX-family, STAT3, STn, TAG-72, transforming growth factor-alpha (TGF-α), transforming growth factor-beta (TGF-β), Thymosin-beta-15, tumor necrosis factor-alpha (“TNF-α”), TRP1, TRP2, tyrosinase, vascular endothelial growth factor (VEGF), ZAG, p16INK4, and glutathione-S-transferase (GST)

6. The combination for use of claim 1, wherein the MVA is MVA-BN or a derivative of MVA-BN.

7. The combination for use of claim 1, wherein a) is administered at the same time as or prior to an administration of b).

8. The combination for use of claim 1, wherein a) and b) are administered to the cancer patient in a priming administration followed by one or more boosting administrations of a) and b) to the cancer patient.

9. The combination for use of claim 1, wherein the cancer patient is suffering from and/or is diagnosed with a cancer selected from the group consisting of: breast cancer, lung cancer, head and neck cancer, thyroid, melanoma, gastric cancer, bladder cancer, kidney cancer, liver cancer, melanoma, pancreatic cancer, prostate cancer, ovarian cancer, or colorectal cancer.

10. The combination for use of claim 9, wherein the breast cancer is a HER2 overexpressing breast cancer.

11. A method for reducing tumor size and or increasing survival in a cancer patient, the combination comprising:
a) administering to the cancer patient a recombinant modified Vaccinia Ankara (MVA) virus comprising a first nucleic acid encoding a tumor-associated antigen (TAA) and a second nucleic acid encoding CD40 Ligand (CD40L), that when administered intravenously induces both an enhanced Natural Killer (NK) cell response and an enhanced T cell response as compared to an NK cell response and a T cell response induced by a non-intravenous administration of a recombinant MVA virus comprising a first nucleic acid encoding a TAA and a second nucleic acid encoding CD40L; and
b) administering to the cancer patient at least one antagonist or agonist of an immune checkpoint molecule;
wherein (a) and (b) are to be administered as a combination treatment; and wherein administration of a) and b) to the cancer patient reduces tumor size and/or increases the survival rate of the cancer patient as compared to a non-IV administration of a) or an administration of b) alone.

12. The method of claim 11, wherein the antagonist or agonist of an immune checkpoint molecule comprises a CTLA-4 antagonist, a PD-1 antagonist, a PD-L1 antagonist, a LAG-3 antagonist, a TIM-3 antagonist, or an ICOS agonist.

13. The method of claim 12, wherein the CTLA-4 antagonist is a CTLA-4 antibody, the PD-1 antagonist is a PD-1 antibody, the PD-L1 antagonist is a PD-L1 antibody, the LAG-3 antagonist is a LAG-3 antibody, the TIM-3 antagonist is a TIM-3 antibody, and the ICOS agonist is an ICOS antibody.

14. The method of claim 11, wherein the TAA is selected from the group consisting of: carcinoembryonic antigen (CEA), Mucin 1, cell surface associated (MUC-1), Prostatic Acid Phosphatase (PAP), Prostate Specific Antigen (PSA), human epidermal growth factor receptor 2 (HER2), survivin, tyrosine related protein 1 (TRP1), tyrosine related protein 2 (TRP2), Brachyury antigen, or combinations thereof.

15. The method of claim 11, wherein the TAA is selected from the group consisting of: 5-α-reductase, α-fetoprotein (AFP), AM-1, APC, April, B melanoma antigen gene (BAGE), β-catenin, Bcl12, bcr-ab1, Brachyury, CA-125, caspase-8 (CASP-8), Cathepsins, CD19, CD20, CD21/complement receptor 2 (CR2), CD22/BL-CAM, CD23/FcεRII, CD33, CD35/complement receptor 1 (CR1), CD44/PGP-1, CD45/leucocyte common antigen (LCA), CD46/membrane cofactor protein (MCP), CD52/CAMPATH-1, CD55/decay accelerating factor (DAF), CD59/protectin, CDC27, CDK4, carcinoembryonic antigen (CEA), c-myc, cyclooxygenase-2 (cox-2), deleted in colorectal cancer gene (DCC), DcR3, E6/E7, CGFR, EMBP, Dna78, farnesyl transferase, fibroblast growth factor-8a (FGF8a), fibroblast growth factor-8b (FGF8b), FLK-1/KDR, folic acid receptor, G250, G melanoma antigen gene family (GAGE-family), gastrin 17, gastrin-releasing hormone, ganglioside 2 (GD2)/ganglioside 3 (GD3)/ganglioside-monosialic acid-2 (GM2), gonadotropin releasing hormone (GnRH), UDP-GlcNAc:R1Man(a1-6)R2 [GlcNAc to Man(α1-6)] β1,6-N¬-acetylglucosaminyltransferase V (GnT V), GP1, gp100/Pme117, gp-100-in4, gp15, gp75/tyrosine-related protein-1 (gp75/TRP1), human chorionic gonadotropin (hCG), heparanase, HER2, human mammary tumor virus (HMTV), 70 kiloDalton heat-shock protein (“HSP70”), human telomerase reverse transcriptase (hTERT), insulin-like growth factor receptor-1 (IGFR-1), interleukin-13 receptor (IL-13R), inducible nitric oxide synthase (“iNOS”), Ki67, KIAA0205, K-ras, H-ras, N-ras, KSA, LKLR-FUT, melanoma antigen-encoding gene 1 (MAGE-1), melanoma antigen-encoding gene 2 (MAGE-2), melanoma antigen-encoding gene 3 (MAGE-3), melanoma antigen-encoding gene 4 (MAGE-4), mammaglobin, MAP17, Melan-A/melanoma antigen recognized by T-cells-1 (MART-1), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (PDGF), μPA, PRAME, probasin, progenipoietin, prostate-specific antigen (PSA), prostate-specific membrane antigen (PSMA), RAGE-1, Rb, RCAS1, SART-1, SSX-family, STAT3, STn, TAG-72, transforming growth factor-alpha (TGF-α), transforming growth factor-beta (TGF-β), Thymosin-beta-15, tumor necrosis factor-alpha (“TNF-α”), TRP1, TRP2, tyrosinase, vascular endothelial growth factor (VEGF), ZAG, p16INK4, and glutathione-S-transferase (GST)

16. The method of claim 11, wherein the MVA is MVA-BN or a derivative of MVA-BN.

17. The method of claim 11, wherein a) is administered at the same time as or prior to b).

18. The method of claim 11, wherein a) and b) are administered to the cancer patient in a priming administration followed by one or more boosting administrations of a) and b) to the cancer patient.

19. The method of claim 11, wherein the cancer patient is suffering from and/or is diagnosed with a cancer selected from the group consisting of: breast cancer, lung cancer, head and neck cancer, thyroid, melanoma, gastric cancer, bladder cancer, kidney cancer, liver cancer, melanoma, pancreatic cancer, prostate cancer, ovarian cancer, or colorectal cancer.

20. A combination therapy for reducing tumor size and or increasing survival in a cancer patient, the combination comprising:
a) a recombinant modified Vaccinia Ankara (MVA) virus comprising a first nucleic acid encoding a tumor-associated antigen (TAA) and a second nucleic acid encoding CD40 Ligand (CD40L), that when administered intravenously induces both an enhanced Natural Killer (NK) cell response and an enhanced T cell response as compared to an NK cell response and a T cell response induced by a non-intravenous administration of a recombinant MVA virus comprising a first nucleic acid encoding a TAA and a second nucleic acid encoding CD40L; and
b) at least one antagonist or agonist of an immune checkpoint molecule;
wherein (a) and (b) are to be administered as a combination treatment; and wherein administration of a) and b) to the cancer patient reduces tumor size and/or increases the survival rate of the cancer patient as compared to a non-IV administration of a) or an administration of b) alone.

21. The combination therapy of claim 20, wherein the antagonist or agonist of an immune checkpoint molecule comprises a CTLA-4 antagonist, a PD-1 antagonist, a PD-L1 antagonist, a LAG-3 antagonist, a TIM-3 antagonist, or an ICOS agonist.

22. The combination therapy of claim 20, wherein the CTLA-4 antagonist is a CTLA-4 antibody, the PD-1 antagonist is a PD-1 antibody, the PD-L1 antagonist is a PD-L1 antibody, the LAG-3 antagonist is a LAG-3 antibody, the TIM-3 antagonist is a TIM-3 antibody, and the ICOS agonist is an ICOS antibody.

23. The combination therapy of claim 20, wherein the TAA is selected from the group consisting of: carcinoembryonic antigen (CEA), Mucin 1, cell surface associated (MUC-1), Prostatic Acid Phosphatase (PAP), Prostate Specific Antigen (PSA), human epidermal growth factor receptor 2 (HER2), survivin, tyrosine related protein 1 (TRP1), tyrosine related protein 2 (TRP2), Brachyury antigen, or combinations thereof.

24. The combination therapy of claim 20, wherein the TAA is selected from the group consisting of: 5-α-reductase, α-fetoprotein (AFP), AM-1, APC, April, B melanoma antigen gene (BAGE), β-catenin, Bcl12, bcr-ab1, Brachyury, CA-125, caspase-8 (CASP-8), Cathepsins, CD19, CD20, CD21/complement receptor 2 (CR2), CD22/BL-CAM, CD23/FcεRII, CD33, CD35/complement receptor 1 (CR1), CD44/PGP-1, CD45/leucocyte common antigen (LCA), CD46/membrane cofactor protein (MCP), CD52/CAMPATH-1, CD55/decay accelerating factor (DAF), CD59/protectin, CDC27, CDK4, carcinoembryonic antigen (CEA), c-myc, cyclooxygenase-2 (cox-2), deleted in colorectal cancer gene (DCC), DcR3, E6/E7, CGFR, EMBP, Dna78, farnesyl transferase, fibroblast growth factor-8a (FGF8a), fibroblast growth factor-8b (FGF8b), FLK-1/KDR, folic acid receptor, G250, G melanoma antigen gene family (GAGE-family), gastrin 17, gastrin-releasing hormone, ganglioside 2 (GD2)/ganglioside 3 (GD3)/ganglioside-monosialic acid-2 (GM2), gonadotropin releasing hormone (GnRH), UDP-GlcNAc:R1Man(a1-6)R2 [GlcNAc to Man(α1-6)] β1,6-N¬-acetylglucosaminyltransferase V (GnT V), GP1, gp100/Pme117, gp-100-in4, gp15, gp75/tyrosine-related protein-1 (gp75/TRP1), human chorionic gonadotropin (hCG), heparanase, HER2, human mammary tumor virus (HMTV), 70 kiloDalton heat-shock protein (“HSP70”), human telomerase reverse transcriptase (hTERT), insulin-like growth factor receptor-1 (IGFR-1), interleukin-13 receptor (IL-13R), inducible nitric oxide synthase (“iNOS”), Ki67, KIAA0205, K-ras, H-ras, N-ras, KSA, LKLR-FUT, melanoma antigen-encoding gene 1 (MAGE-1), melanoma antigen-encoding gene 2 (MAGE-2), melanoma antigen-encoding gene 3 (MAGE-3), melanoma antigen-encoding gene 4 (MAGE-4), mammaglobin, MAP17, Melan-A/melanoma antigen recognized by T-cells-1 (MART-1), mesothelin, MIC A/B, MT-MMPs, mucin, testes-specific antigen NY-ESO-1, osteonectin, p15, P170/MDR1, p53, p97/melanotransferrin, PAI-1, platelet-derived growth factor (PDGF), μPA, PRAME, probasin, progenipoietin, prostate-specific antigen (PSA), prostate-specific membrane antigen (PSMA), RAGE-1, Rb, RCAS1, SART-1, SSX-family, STAT3, STn, TAG-72, transforming growth factor-alpha (TGF-α), transforming growth factor-beta (TGF-β), Thymosin-beta-15, tumor necrosis factor-alpha (“TNF-α”), TRP1, TRP2, tyrosinase, vascular endothelial growth factor (VEGF), ZAG, p16INK4, and glutathione-S-transferase (GST)

25. The combination therapy of claim 20, wherein the MVA is MVA-BN or a derivative of MVA-BN.

26. The combination therapy of claim 20, wherein a) is administered at the same time as or prior to b).

27. The combination therapy of claim 20, wherein a) and b) are administered to the cancer patient in a priming administration followed by one or more boosting administrations of a) and b) to the cancer patient.

28. The combination therapy of claim 16, wherein the cancer patient is suffering from and/or is diagnosed with a cancer selected from the group consisting of: breast cancer, lung cancer, head and neck cancer, thyroid, melanoma, gastric cancer, bladder cancer, kidney cancer, liver cancer, melanoma, pancreatic cancer, prostate cancer, ovarian cancer, or colorectal cancer.
